(19) United States
(12) Patent Application Publication
(10) Pub. No.: US 2019/0127389 A1
(43) Pub. Date: May 2, 2019

(54) Processes And Oxazolidine-Containing Intermediates For The Preparation Of Morphine Analogs And Derivatives

(71) Applicant: Noramco, Inc., Athens, GA (US)

(72) Inventors: Christopher Oliver Kappe, Graz (AT); Bernhard Gutmann, Graz (AT); Ulrich Weigl, Hilzingen (DE); Patrick Egli, Eglisau (CH); Douglas Phillip Cox, Eagleville, PA (US); David Cantillo, Graz (AT)

(73) Assignee: Noramco, Inc., Athens, GA (US)

(21) Appl. No.: 16/095,640

(22) PCT Filed: Apr. 21, 2017
(86) PCT No.: PCT/US2017/028894

§ 371 Date: Oct. 22, 2018

Related U.S. Application Data
(60) Provisional application No. 62/411,174, filed on Oct. 21, 2016.
(60) Provisional application No. 62/410,621, filed on Oct. 20, 2016.
(60) Provisional application No. 62/326,090, filed on Apr. 22, 2016.

(51) Int. Cl. C07D 489/08

(52) U.S. Cl. CPC C07D 489/08

(57) ABSTRACT

The present invention relates to processes useful in the preparation of morphine analogs and derivatives, such as naltrexone, naloxone and nalbuphine and intermediates in the synthesis of said morphine analogs and derivatives. In a particular example, the process begins with for example oxymorphone, oxycodone, 14-hydroxycodeinone or 14-hydroxymorphinone, and includes the formation of an oxazolidine-containing intermediate using catalytic oxidation.

### CROSS REFERENCE TO RELATED APPLICATIONS

This application claims the benefit of U.S. Provisional Application No. 62/326,090, filed Apr. 22, 2016, U.S. Provisional Application No. 62/410,621, filed Oct. 20, 2016, and U.S. Provisional Application No. 62/411,174, filed Oct. 21, 2016. The contents of each of these applications are incorporated herein by reference in their entireties.

### FIELD OF THE INVENTION

The present disclosure relates to processes useful in the preparation of morphine analogs and derivatives, such as naltrexone, naloxone and nalbuphine and intermediates in the synthesis of said morphine analogs and derivatives. In a particular example, the process may begin with oxymorphone, oxycodone, 14-hydroxycodeinone or 14-hydroxymorphinone, and may include the formation of an oxazolidine-containing intermediate using catalytic oxidation.

### BACKGROUND OF THE INVENTION

Various morphine antagonists such as naltrexone, naloxone, and nalbuphine are available by semi-synthesis from the natural opiates such as morphine, codeine, thebaine or oripavine, as shown in the structures below.

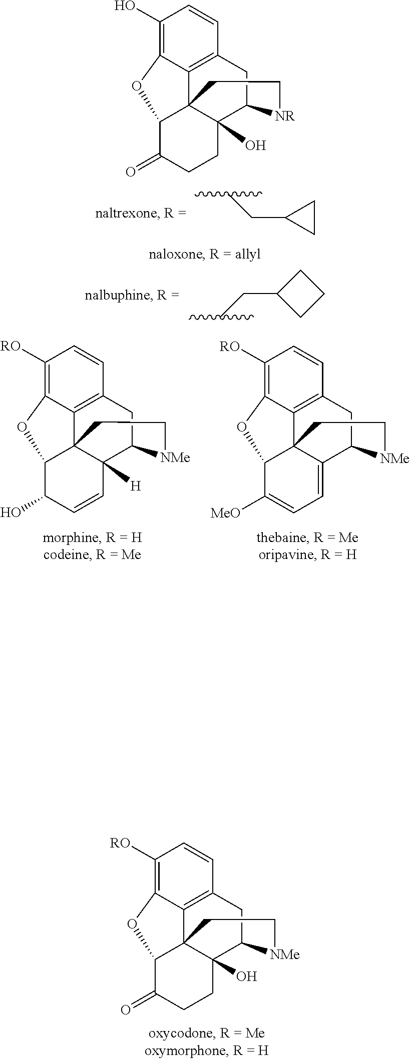

These compounds are used extensively in medicine as antagonists (e.g., naltrexone and naloxone) and mixed agonist/antagonist (e.g., nalbuphine). Naltrexone has long been used for the treatment of alcoholism, and is the active ingredient in Vivitrol®, an extended release injectable suspension for the treatment of alcoholism and opioid dependence. Naloxone is the active ingredient in Narcan® for the reversal of opioid overdose and is used to mitigate side effects in combination with buprenorphine (Suboxone®) for the treatment of opioid addiction, with tilidine (Valoron N®) for the treatment of pain and with oxycodone (Targin®) for the prophylaxis and/or treatment of opioid-induced bowel dysfunction during the treatment of pain. Nalbuphine is the active ingredient in Nubain® and is used for the treatment of pain in very low doses particularly in women.

The introduction of the C-14 hydroxyl into various natural morphinans to produce oxycodone and oxymorphone has been reduced to practice on large scales with a high degree of efficiency by oxidation of thebaine or oripavine. Methods for direct C—H oxidation at C-14 for compounds such as codeine, morphine, or hydrocodone have been reported but are not very efficient or practical at this time. On the other hand, N-demethylation of natural opiates still represents a challenge, especially in terms of efficiency or the focus on environmentally benign procedures and reagents. Many methods have been employed for the demethylation; these include the use of cyanogen bromide (von Braun reaction) (von BRAUN, J.; Chem. Ber., 1900, pp. 1438, Vol. 33), methyl or ethyl chloroformate (COOLEY, J. H.; EVAIN, E. J.; Synthesis 1989, p. 1), 1-chloroethyl chloroformate (ACE-Cl) (OLOFSON, R. A.; et al., J. Org. Chem. 1984, p. 2081, Vol. 49), and microbial protocols ((a) MADYSTHA, K. M., Proc. Indian Acad. Sci. 1994, 106, 1203; (b) MADYSTHA, K. M., et al., J. Chem. Soc. Perkin Trans. 1 1994, p. 911), including a recently published procedure employing fungal biotransformations (CHAUDHARY, V, et al., Collect. Czech. Chem. Commun. 2009, pp. 1179-1193, Vol. 74). The biotransformations of several morphine alkaloids with the strain Cunninghamella echinulata and several others produced the free amines in reasonable yields and purity. Such processes, when scaled up and improved by the creation of a transgenic vector that would express the required fungal cytochrome in an E. coli carrier would have great potential as an environmentally benign N-demethylation protocol.

N-Demethylation/acylation of hydrocodone and tropane alkaloids was also accomplished via palladium catalysts that provided N-acetylhydrocodone and other acyl derivatives (CARROLL, R. J. et al. Adv. Synth. Catal. 2008, 350, 2984; CARROLL, R. J. et al. U.S. Patent Application Publication No. US 2009/0005565). N-Demethylation/acylation of 14-hydroxymorphinan derivatives was also accomplished via an intramolecular functional group transfer from the C-14 hydroxyl to the N-17 nitrogen atom following a palladium-catalyzed N-demethylation as shown in Scheme 1 (HUDLICKY, T and MACHARA, A. U.S. Pat. No. 8,946,214; (b) MACHARA, A et al., Advanced Synthesis & Catalysis (2012), pp. 613-26, Vol. 354(4)).

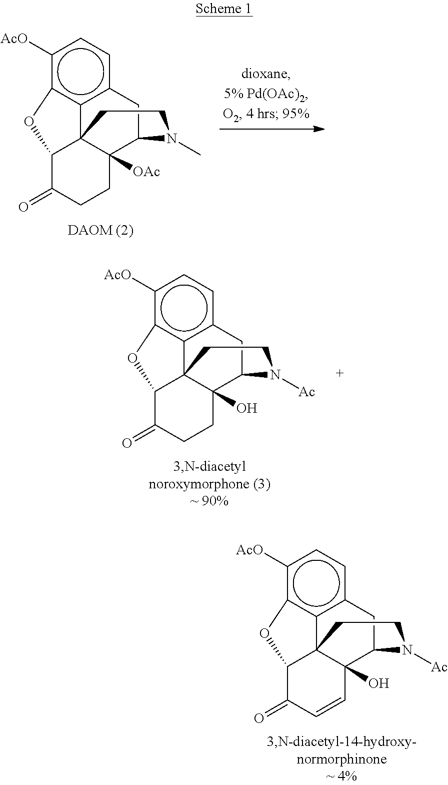

Recently, iron (II) as well as iron (0) catalyzed N-demethylation of several morphinan N-oxides was reported by Scammells (KOK, G., et al., Adv. Synth. Catal. 2009, p. 283, Vol. 351; DONG, Z., et al., J. Org. Chem. 2007, p. 9881, Vol. 72). Smith et al. in PCT Patent Publication WO 2005/02848 developed a method to convert N-methylated 6-oxo-14-hydroxymorphinanes to the corresponding nor compounds by treating the corresponding N-oxide with a Fe(II) based reducing agent in the presence of formic acid to form an oxazolidine structure. The oxazolidine can then be converted to the corresponding nor-morphinane by acid hydrolysis, as shown in Scheme 2.

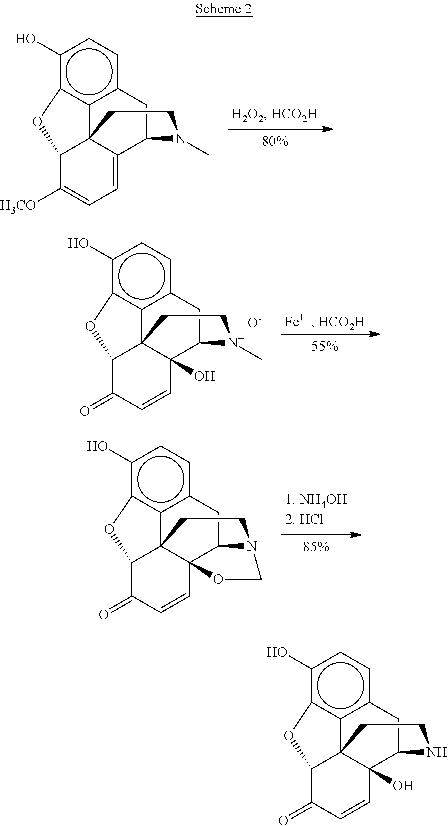

Conversion of the N-oxide to the corresponding oxazolidine works equally well whether the 7, 8 carbon bond is unsaturated or saturated, as shown with oxymorphone, in Scheme 3, below.

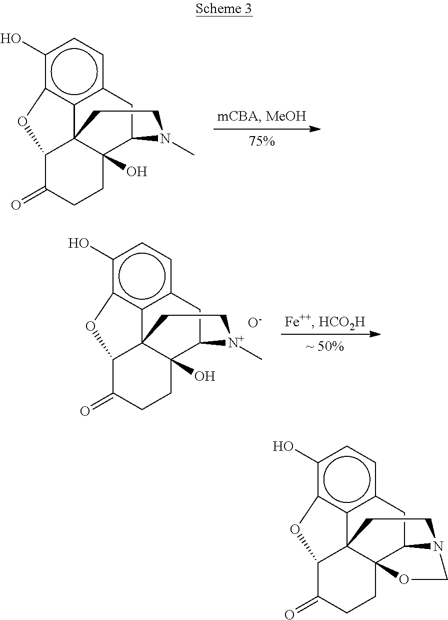

Hudlicky and co-workers (HUDLICKY, T., et al., U.S. Pat. No. 8,957,072; WERNER, L., et al., Advanced Synthesis & Catalysis (2012), pp. 2706-2712, Vol. 354(14-15)) reported that the N-oxide of oxymorphone is easily demethylated with the Burgess reagent and that the intermediate iminium ion is trapped to form oxazolidine containing compounds in excellent yield and in a one-pot sequence from oxymorphone as shown in Scheme 4.

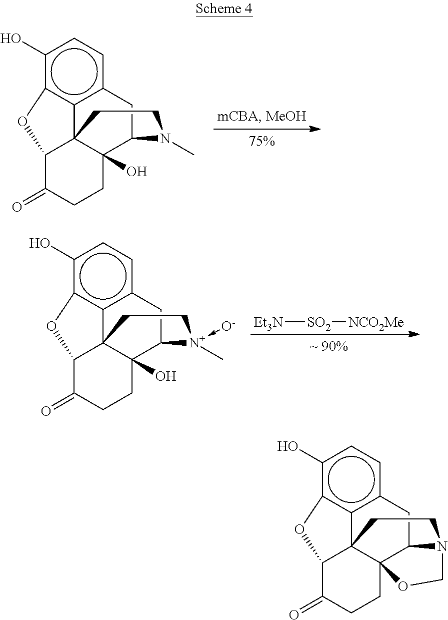

They also showed that the oxazolidine containing compounds could be reacted further with cyclopropylmethylbromide to form the corresponding quaternary oxazolidine salt containing intermediate, which in turn could be hydrolysed under mild conditions to form naltrexone in high yields as shown in Scheme 5.

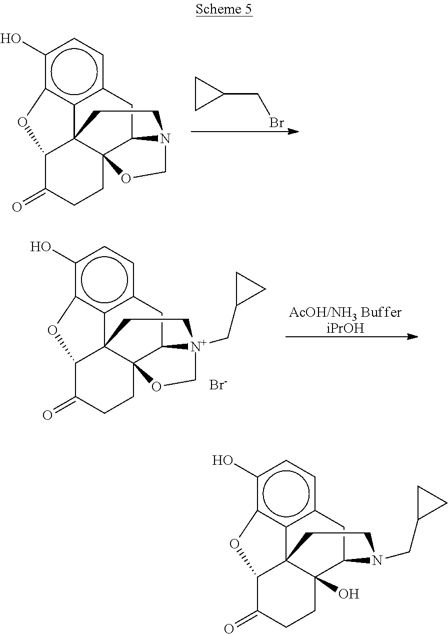

Pd catalyzed oxidation using oxygen of natural and semi-synthetic morphinanones produces nor-morphinanes by N-demethylation of the 17-N methyl group. It was previously reported that analogous N-demethylation of the oxycodone, a 14-hydroxymorphinan, does not occur unless the 14-hydroxy group is first protected by an acyl protecting group (MACHARA, A., et al., Advanced Synthesis & Catalysis (2012), pp. 2713-1718, Vol. 354(14-15)).

There remains a need to provide a direct conversion of 14-hydroxymorphinanones to the corresponding analogs via N-demethylation and alkylation.

### SUMMARY OF THE INVENTION

The present disclosure is directed to a process including the oxidation reaction (e.g., in the presence of Pd and/or Pt catalyst with oxygen or peroxides) of 14-hydroxymorphinanones such as, for example, 14-hydroxymorphinone, 14-hydroxycodeinone, oxymorphone, oxycodone and derivatives of these, to yield the corresponding oxazolidine-containing intermediate, and conversion of said oxazolidine-containing intermediate to yield naltrexone, nalbuphine, naloxone, and other analogs or derivatives.

The present application is directed to a process for the preparation of compounds of Formula I:

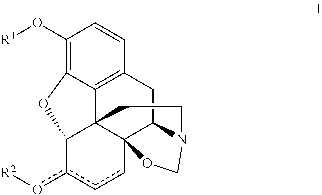

wherein

each [symbol] represents a single or double bond; provided that two double bonds are not adjacent to each other;

R¹ is selected from the group consisting of H, C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;

R² is selected from the group consisting of C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;

provided that when “ [symbol] ” represents O═, then R² is absent;

wherein one or more hydrogen atoms on the R¹ and R² groups is optionally replaced with a halogen, e.g., F;

including the steps of

providing a compound of formula II;

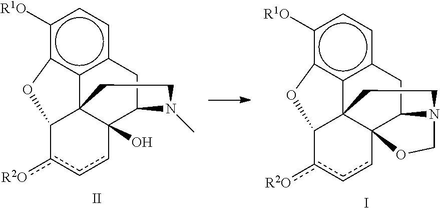

reacting the compound of Formula II with an oxidizing agent; in the presence of a metal catalyst; in a solvent or mixture of solvents; to yield the corresponding compound of Formula I. The compounds of Formula I are useful for the preparation of morphine analogs or derivatives.

The present application is further directed to a process for the preparation of compounds of Formula III

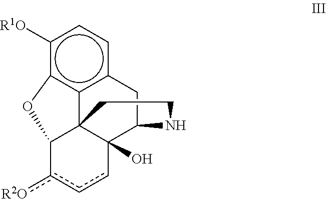

wherein

each [symbol] represents a single or double bond; provided that two double bonds are not adjacent to each other;

R¹ is selected from the group consisting of H, C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;

R² is selected from the group consisting of H, C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;

provided that when “ [symbol] ” represents O═, then R² is absent;

wherein one or more available hydrogens on the R¹ and R² groups is optionally replaced with a halogen, e.g., F;

including the steps of

providing a compound of formula II;

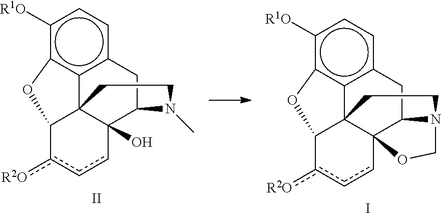

reacting a compound of Formula II with an oxidizing agent; in the presence of a metal catalyst; in a solvent or mixture of solvents; to yield the corresponding compound of Formula I;

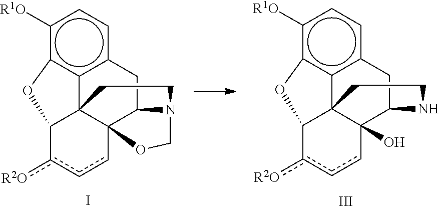

hydrolyzing the compound of Formula I under acidic or basic conditions; in a solvent or mixture of solvents; to yield the corresponding compound of Formula III.

One skilled in the art will recognize that wherein the compound of Formula I, R¹ and/or R² are oxygen protecting group(s) which can be removed under hydrolysis conditions, then in the resulting compound of Formula III, the corresponding R¹ and/or R² are groups are hydrogen.

The present application is further directed to a process for the preparation of compounds of Formula VI

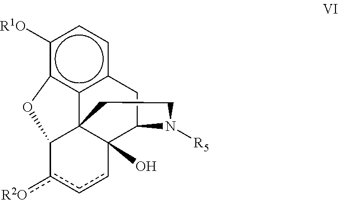

wherein

each [symbol] represents a single or double bond; provided that two double bonds are not adjacent to each other;

R¹ is selected from the group consisting of H, C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;

R² is selected from the group consisting of H, C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;

provided that when “ [symbol] ” represents O═, then R² is absent;

R⁵ is selected from the group consisting of C₃₋₁₀cycloalkyl, C₃₋₁₀cycloalkenyl, C₁₋₁₀alkyl, C₂₋₁₀alkenyl, C₆₋₁₀aryl, C₁₋₁₀alkyleneC₆₋₁₀aryl and C₁₋₁₀alkyleneC₃₋₁₀cycloalkyl;

wherein one or more available hydrogens on the R³ and R⁴ groups is optionally replaced with a halogen, e.g., F;

including the steps of

providing a compound of formula II;

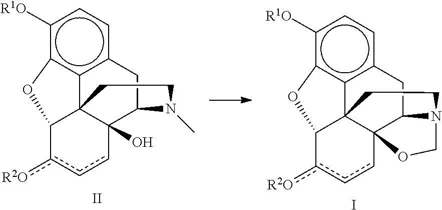

reacting the compound of Formula II with an oxidizing agent; in the presence of a metal catalyst; in a solvent or mixture of solvents; to yield the corresponding compound of Formula I;

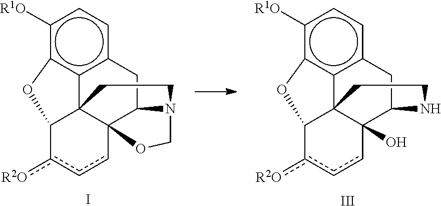

hydrolyzing the compound of Formula I under acidic or basic conditions; in a solvent or mixture of solvents; to yield the corresponding compound of Formula III;

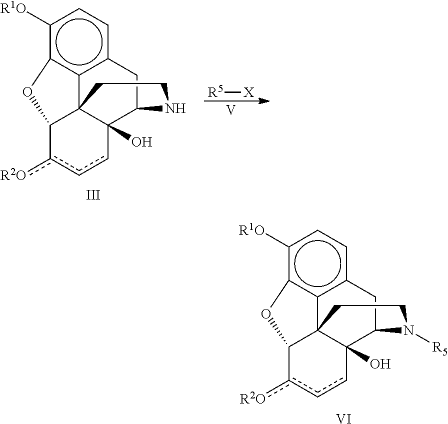

reacting the compound of Formula III with a compound of Formula V, i.e., a compound of Formula R⁵—X, wherein X is a leaving group (counteranion), to selectively alkylate the compound of Formula III at the 17-N position; in a solvent or mixture of solvents; to yield the corresponding compound of Formula VI.

The present application is further directed to a process for the preparation of compounds of Formula IV

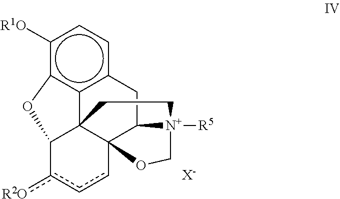

wherein

each [symbol] represents a single or double bond; provided that two double bonds are not adjacent to each other;

R¹ is selected from the group consisting of H, C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;

R² is selected from the group consisting of C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;

provided that when “ [symbol] ” represents O═, then R² is absent;

R⁵ is selected from the group consisting of C₃₋₁₀cycloalkyl, C₃₋₁₀cycloalkenyl, C₁₋₁₀alkyl, C₂₋₁₀alkenyl, C₆₋₁₀aryl, C₁₋₁₀alkyleneC₆₋₁₀aryl and C₁₋₁₀alkyleneC₃₋₁₀cycloalkyl;

X is a counteranion;

wherein one or more hydrogen atoms on R¹, R² and R⁵ is optionally replaced with a halogen, e.g., F;

including the steps of

providing a compound of formula II;

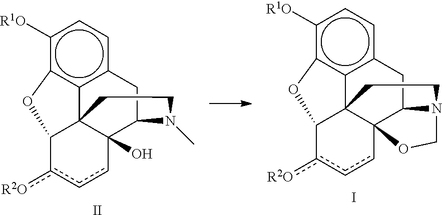

reacting the compound of Formula II with an oxidizing agent; in the presence of a metal catalyst; in a solvent or mixture of solvents; to yield the corresponding compound of Formula I;

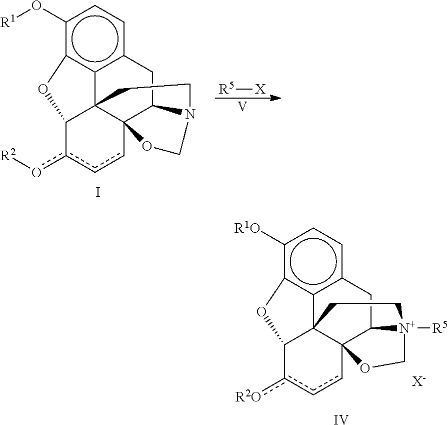

reacting a compound of Formula I with a compound of Formula V, wherein X is a leaving group (counteranion); in a solvent or mixture of solvents; to yield the corresponding compound of Formula IV.

The present application is further directed to a process for the preparation of compounds of Formula VI

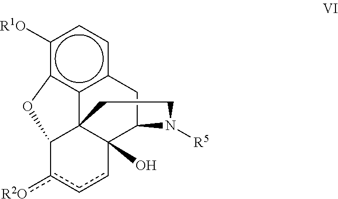

wherein

each [symbol] represents a single or double bond; provided that two double bonds are not adjacent to each other;

R¹ is selected from the group consisting of H, C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;

R² is selected from the group consisting of H, C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;

provided that when “ [symbol] ” represents O═, then R² is absent;

R⁵ is selected from the group consisting of C₃₋₁₀cycloalkyl, C₃₋₁₀cycloalkenyl, C₁₋₁₀alkyl, C₂₋₁₀alkenyl, C₆₋₁₀aryl, C₁₋₁₀alkyleneC₆₋₁₀aryl and C₁₋₁₀alkyleneC₃₋₁₀cycloalkyl;

wherein one or more available hydrogens on the R³ and R⁴ groups is optionally replaced with a halogen, e.g., F;

including the steps of

providing a compound of formula II;

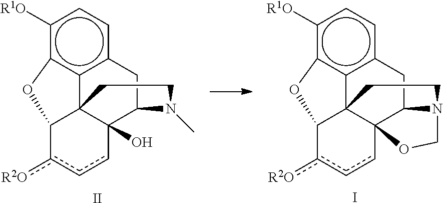

reacting the compound of Formula II with an oxidizing agent; in the presence of a metal catalyst; in a solvent or mixture of solvents; to yield the corresponding compound of Formula I

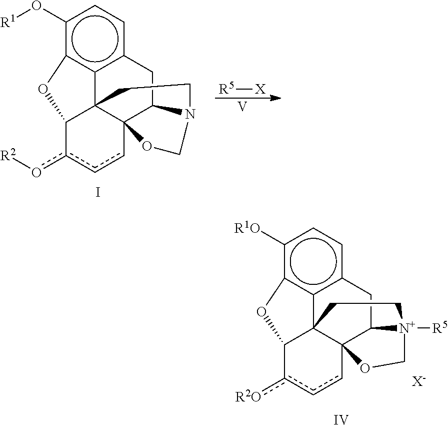

reacting a compound of Formula I with a compound of Formula V, wherein X is a leaving group (counteranion); in a solvent or mixture of solvents; to yield the corresponding compound of Formula IV;

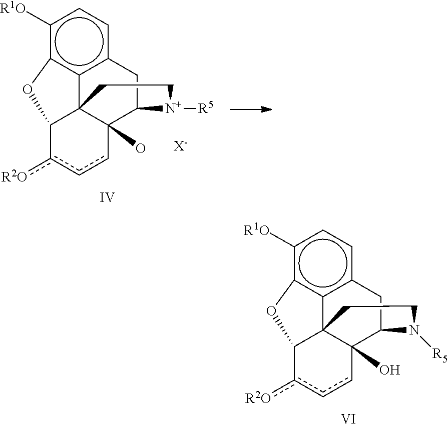

hydrolyzing the compound of Formula IV, under acidic or basic conditions; in a solvent or mixture of solvents; to yield the corresponding compound of Formula VI.

One skilled in the art will recognize that wherein the compound of Formula IV, R¹ and/or R² is a protecting groups which may be removed under hydrolysis conditions, then in the corresponding compound of Formula VI, the corresponding R¹ and/or R² are hydrogen.

The present disclosure is further directed to a process for the preparation of compounds of Formula VII

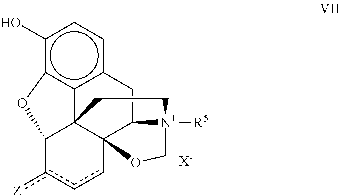

wherein

each [symbol] represents a single or double bond; provided that two double bonds are not adjacent to each other;

Z is selected from the group consisting of O and OH; provided that when [symbol] is Z═, then Z is O; provided further that when [symbol] is Z—, then Z is OH;

R⁵ is selected from the group consisting of C₃₋₁₀cycloalkyl, C₃₋₁₀cycloalkenyl, C₁₋₁₀alkyl, C₂₋₁₀alkenyl, C₆₋₁₀aryl, C₁₋₁₀alkyleneC₆₋₁₀aryl and C₁₋₁₀alkyleneC₃₋₁₀cycloalkyl;

X is a counteranion;

wherein one or more hydrogens on R³ is optionally replaced with a halogen, e.g., F;

including the steps of

providing a compound of formula II;

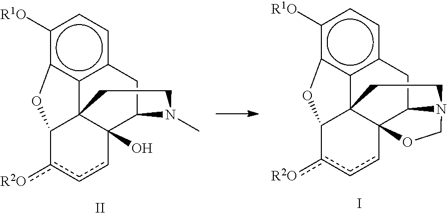

reacting the compound of Formula II with an oxidizing agent; in the presence of a metal catalyst; in a solvent or mixture of solvents; to yield the corresponding compound of Formula I

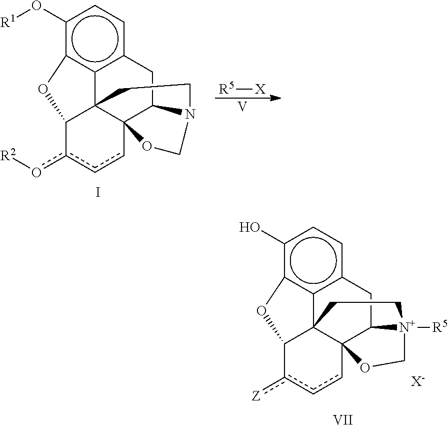

reacting a compound of Formula I with a compound of Formula V, wherein X is a leaving group (counteranion); in a solvent or mixture of solvents; to yield the corresponding compound of Formula VII.

In certain embodiments of the present disclosure, one or more process steps as described herein are run in a continuous flow reaction.

In other embodiments, the metal catalyst can be a palladium or platinum catalyst configured to exist in either a +2 oxidation state or a 0 oxidation state. The reaction can occur in the presence of an alcohol configured to convert a palladium or platinum catalyst from a +2 oxidation state to a 0 oxidation state. The alcohol can be configured to, or can be capable of, regenerating an active catalyst (e.g., Pd⁰) from an inactive catalyst (e.g., Pd²). An inactive catalyst can form during, for example, the oxidation step. The alcohol can be a C₁₋₁₀ primary or secondary alcohol, such as ethylene glycol or 2-propanol.

The present disclosure is further directed to a product prepared according to any of the processes described herein.

Other features and advantages of the present application will become apparent from the following detailed description. It should be understood, however, that the detailed description and the specific examples which follow hereinafter, while indicating embodiments of the application, are given by way of illustration only, since various changes and modifications within the spirit and scope of the application will become apparent to those skilled in the art from this detailed description.

### BRIEF DESCRIPTION OF THE FIGURES

The foregoing and other advantages provided by the present disclosure will be more fully understood from the following description of exemplary embodiments when read together with the accompanying drawings, in which:

FIG. 1 illustrates a packed bed reactor set-up, as used in the experiment of Example 2.

FIG. 2 illustrates a flow reactor for gas/liquid reactions, as used in the experiment of Example 3.

FIG. 3 illustrates a flow reactor for gas/liquid reactions, as used in the experiment of Example 5.

FIG. 4 illustrates the HPLC-UV/Vis chromatogram of the product containing reaction mixture of Example 7.

FIG. 5 illustrates the HPLC-UV chromatogram (205 nm) of the isolated product (7) of Example 9.

FIG. 6 illustrates the HPLC-UV chromatogram (215 nm) of the isolated product after derivatization with acetic anhydride described in Example 10.

FIG. 7 illustrates a process scheme for the preparation of 1,3-oxazolidine by hydrogenation/oxidative cyclization catalyzed by colloidal Pd(0).

FIG. 8 illustrates a hydrogenation/oxidative cyclization synthetic sequence for the preparation of 1,3-oxazolidine using heterogeneous catalysts.

FIG. 9 illustrates HPLC chromatograms (205 nm) for the crude reaction mixtures after the hydrogenation step (HPLC trace (a)), and the continuous flow oxidative cyclization step (HPLC trace (b)).

FIG. 10 illustrates HPLC monitoring (205 nm) of the continuous flow oxidation of compound 6 (Example 15) after the reaction mixture was processed multiple times.

FIG. 11(a) illustrates the structure of the SiliaCat DPP-Pd catalyst.

FIG. 11(b) illustrates the preactivation of the catalyst in the packed-bed reactor.

FIG. 12 illustrates the HPLC monitoring (205 nm) for the continuous flow oxidation of 6 to 7 (Example 15) using ethylene glycol as additive; FIG. 12(a): 10% EG in DMA; FIG. 12(b): 20% EG in DMA; 10 mL reaction volume was processed for each experiment.

FIG. 13 illustrates the HPLC chromatogram (205 nm) for the continuous flow synthesis of 7 (Example 15) using a packed bed reactor containing SiliaCat DPP-Pd (using DMA/EG 8:2 as solvent).

FIG. 14 illustrates the HPLC monitoring (205 nm) for the continuous flow oxidation of 6 to 7 during a 50 mL run.

FIG. 15 illustrates the HPLC monitoring (205 nm) of the continuous flow oxidation of 6 (Example 15) after the reaction mixture was processed multiple times under different conditions; FIG. 15(a): 100° C., 1 equiv O₂, 5 bar, 3 mol % Pd(OAc)₂; FIG. 15(b): 100° C., 0.5 equiv O₂, 3 bar, 3 mol % Pd(OAc)₂; FIG. 15(c): 100° C., 1 equiv O₂, 3 bar, 3 mol % Pd(OAc)₂; FIG. 15(d): 80° C., 1 equiv O₂, 3 bar, 3 mol % Pd(OAc)₂.

FIG. 16 illustrates the HPLC monitoring for the continuous oxidation of 6 (Example 15) to 7 using a packed bed reactor containing 220 mg SiliaCat DPP-Pd and DMA as solvent; rapid decrease in the reaction conversion is observed in each flow run, but the catalytic efficiency can be recovered by treating the solid support with EG/DMA 1:1; conditions: 120° C., 1.5 mol equiv O₂, 0.1 M.

FIG. 17 illustrates the ¹H NMR spectra of 7 (Example 15).

FIG. 18 illustrates the ¹³C NMR spectra of 7 (Example 15).

### DETAILED DESCRIPTION OF THE INVENTION

The present disclosure is directed a process for the preparation of compounds of Formula I

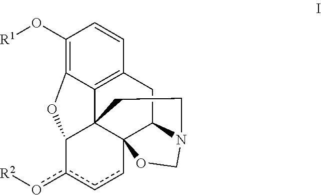

wherein R¹ and R² are as herein defined. The compounds of Formula I are useful as intermediates in the synthesis of morphine analogs and derivatives, including but not limited to naltrexone, nalbuphine and naloxone.

The present disclosure is further directed to processes for the preparation of compounds of Formula III, compounds of Formula IV, compounds of Formula VI and compounds of Formula VII, as herein described in more detail. The compounds of Formula III, Formula IV, Formula VI and Formula VII are useful as intermediates in the synthesis of or are themselves morphine analogs and derivatives, useful for the treatment of for example moderate or severe pain.

### Definitions

Unless otherwise indicated, the definitions and embodiments described in this and other sections are intended to be applicable to all embodiments and aspects of the application herein described for which they are suitable as would be understood by a person skilled in the art.

As used in this application, the singular forms “a”, “an” and “the” include plural references unless the content clearly dictates otherwise. For example, an embodiment including “an oxidizing agent” should be understood to present certain aspects with one oxidizing agent, or two or more additional oxidizing agents.

In embodiments including an “additional” or “second” component, such as an additional or second oxidizing agent, the second component as used herein is chemically different from the other components or first component. A “third” component is different from the other, first, and second components, and further enumerated or “additional” components are similarly different.

The term “suitable” as used herein means that the selection of the particular compound or conditions would depend on the specific synthetic manipulation to be performed, and the identity of the molecule(s) to be transformed, but that the selection would be within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

As used herein, the notation “*” shall denote the presence of a stereogenic center.

Where the compounds according to this disclosure have at least one chiral center, they may accordingly exist as enantiomers. Where the compounds possess two or more chiral centers, they may additionally exist as diastereomers. It is to be understood that all such isomers and mixtures thereof are encompassed within the scope of the present disclosure.

It is to be understood that all such stereo-isomers and mixtures thereof in any proportion are encompassed within the scope of the present application. It is to be further understood that while the stereochemistry of the compounds may be as shown in any given compound listed herein, such compounds may also contain certain amounts (e.g. less than 20%, suitably less than 10%, more suitably less than 5%) of compounds having alternate stereochemistry.

Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, it is intended that within the scope of the present disclosure, any element, in particular when mentioned in relation to a compound used or produced in any of the processes described herein, shall comprise all isotopes and isotopic mixtures of said element, either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. Radiolabelled compounds of formula (I) may comprise a radioactive isotope selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. In an example, one or more atoms on the R¹ and/or R² groups is optionally replaced with an isotopic label as herein described.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed.

The term “counteranion” as used herein refers to a negatively charged species consisting of a single element, or a negatively charged species consisting of a group of elements connected by ionic and/or covalent bonds. Suitable examples include, but are not limited to Cl⁻, Br⁻, I⁻, SO₄²—, CH₃SO₄⁻, TsO⁻, BzO⁻, CO₃²⁻, R—CO₂⁻ (wherein R is for example CF₃, C₁₋₆alkyl, phenyl, and the like), and the like. In certain preferred embodiments, the counteranion is selected from the group consisting of Cl⁻, Br⁻, I⁻, SO₄²—, CH₃SO₄⁻ and TsO⁻, preferably Br⁻.

The term “acyl” as used herein, whether it is used alone or as part of another group, means straight or branched chain, saturated alkyl chain bound through a carbonyl (—C(O)—) groups. The term C₁₋₆acyl means an acyl group having 1, 2, 3, 4, 5, 6 or 7 carbon atoms (i.e. —C(O)—C₁₋₆alkyl). It is an embodiment of the application that, in the acyl groups, one or more, including all of the available hydrogen atoms are optionally replaced with a halogen, e.g., F or ²H and thus include, for example trifluoroacetyl and the like.

The term “alkyl” as used herein, whether it is used alone or as part of another group, means straight or branched chain, saturated carbon chains. The term “C₁₋₆alkyl” means an alkyl group having 1, 2, 3, 4, 5, or 6 carbon atoms. It is an embodiment of the application that, in the alkyl groups, one or more, including all, of the hydrogen atoms are optionally replaced with a halogen, e.g., F or ²H and thus include, for example trifluoromethyl, pentafluoroethyl and the like.

The term “alkylene” as used herein, whether it is used alone or as part of another group, refers to a bivalent alkyl group. For example, the term methylene mean a bivalent group of the formula —CH₂—.

The term “alkenyl” as used herein, whether it is used alone or as part of another group, means straight or branched carbon chain, containing at least one unsaturated double bond. The term C₂₋₆alkenyl means an alkenyl group having 2, 3, 4, 5, or 6 carbon atoms and at least one double bond. It is an embodiment of the application that, in the alkenyl groups, one or more, including all, of the hydrogen atoms are optionally replaced with a halogen, e.g., F or ²H and thus include, for example trifluoroethenyl, pentafluoropropenyl, and the like.

The term “cycloalkyl” as used herein, whether it is used alone or as part of another group, means a cyclic, saturated carbon ring structure. The term “C₃₋₁₀cycloalkyl” means a cycloalkyl group having 3, 4, 5, 6, 7, 8, 9 or 10 carbon atoms. It is an embodiment of the application that, in the cycloalkyl groups, one or more, including all, of the hydrogen atoms are optionally replaced with a halogen, e.g., F or ²H.

The term “cycloalkenyl” as used herein, whether it is used alone or as part of another group, means cyclic, unsaturated carbon ring structure containing at least one unsaturated double bond. The term “C₃₋₁₀cycloalkenyl” means a cycloalkenyl group having 3, 4, 5, 6, 7, 8, 9 or 10 carbon atoms and at least one double bond. It is an embodiment of the application that, in the cycloalkenyl groups, one or more, including all, of the hydrogen atoms are optionally replaced with a halogen, e.g., F or ²H.

The term “aryl” as used herein refers to cyclic groups that contain at least one aromatic ring. In an embodiment of the application, the aryl group contains 6, 9 or 10 atoms, such as phenyl, naphthyl or indanyl. It is an embodiment of the application that, in the aryl groups, one or more, including all, of the hydrogen atoms are optionally replaced with a halogen, e.g., F or ²H and thus include, for example pentafluorophenyl and the like.

The term “halo” as used herein refers to a halogen atom and includes F, Cl, Br and I.

The term “oxidizing agent” as used herein means any compound or combination of compounds that oxidizes a desired functional group(s) but does not otherwise react with or degrade the substrate comprising the functional group(s). An oxidizing agent results in the overall loss of electrons, or in the case of organic chemistry, hydrogen atoms from the functional group. Suitable examples of oxidizing agents include, but are not limited to air, oxygen, t-butylperoxide, and the like. In a preferred embodiment, the oxidizing agent is selected from the group consisting of air and oxygen, more preferably oxygen.

The term “inert solvent” as used herein means a solvent that does not interfere with or otherwise inhibit a reaction. Accordingly, the identity of the inert solvent will vary depending on the reaction being performed. The selection of inert solvent is within the skill of a person in the art. Examples of inert solvents include, but are not limited to, benzene, toluene, tetrahydrofuran, ethyl ether, ethyl acetate, dimethyl formamide (DMF), dimethylacetamide (DMA), N-methylpyrollidone (NMP), acetonitrile, C₁₋₆alkylOH (e.g. methanol, ethanol, n-propanol, 2-propanol, n-butanol, butan-2-ol and 2-methyl-1-propanol), diethylcarbonate, hexane and dimethylsulfoxide (DMSO). Further examples, can include aqueous solutions, such as water and dilute acids and bases, and ionic liquids, provided that such solvents do not interfere with the reaction.

The term “solvent” and “inert solvent” includes both a single solvent and a mixture comprising two or more solvents. Suitable examples of solvents include, but are not limited to 1,4-dioxane, acetonitrile, dimethylsulfoxide (DMSO) dimethylacetamide (DMA), dimethylformamide (DMF) and N-methylpyrrolidone (NMP), and the like. In a preferred embodiment, the solvent is selected from the group consisting of dimethylsulfoxide (DMSO) dimethylacetamide (DMA), dimethylformamide (DMF) and N-methylpyrrolidone (NMP), more preferably dimethylacetamide (DMA).

The term “leaving group” as used herein refers to a group that is readily displaceable by a nucleophile, for example, under nucleophilic substitution reaction conditions. One skilled in the art will further recognize that in certain embodiments of the present disclosure, the “leaving group” corresponds to the X counteranion. Examples of suitable leaving groups include, but are not limited to, halo, Ms, Ts, Ns, Tf, C₁₋₆acyl, and the like. In an embodiment, the leaving group is selected from the group consisting of halo, preferably bromide.

The terms “protective group” or “protecting group” or the like, as used herein, refer to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas). Examples of suitable protecting groups include, but are not limited to t-Boc, Ac, Ts, Ms, silyl ethers such as TMS, TBDMS, TBDPS, Tf, Ns, Bn, Fmoc, dimethoxytrityl, methoxyethoxymethyl ether, methoxymethyl ether, pivaloyl, p-methyoxybenzyl ether, tetrahydropyranyl, trityl, ethoxyethyl ethers, carbobenzyloxy, benzoyl and the like.

As used herein, unless otherwise noted, the term “oxygen protecting group” shall mean a group which may be attached to a oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T.W. Greene & P.G.M. Wuts, Protective Groups in Organic Synthesis, John Wiley & Sons, 1991.

In an embodiment of the present disclosure, R¹ and R² are each an independently selected oxygen protecting group. In another embodiment of the present disclosure, R¹ and R² are each an oxygen protecting group, wherein the R¹ and R² oxygen protecting groups are the same.

In another embodiment of the present disclosure, R¹ and R² are each an independently selected oxygen protecting group selected from the group consisting of benzyl, and acetyl. In another embodiment of the present disclosure, R¹ and R² are each an oxygen protecting group, wherein the R¹ and R² oxygen protecting groups are the same and are selected from the group consisting of benzyl and acetyl. In another embodiment of the present disclosure, R¹ and R² are the same and are acetyl.

Abbreviations used in the specification, particularly the Schemes and Examples, are as follows:
Ac=acetyl
Ac₂O=acetic anhydride
AcOH=acetic acid
BOC or t-Boc=t-butyloxycarbonyl
Bn or Bz=benzyl
DMA=dimethylacetamide
DMF=dimethylformamide
DMSO═dimethylsulfoxide
Fmoc=fluorenylmethoxycarbonyl
HPLC=high performance liquid chromatography
¹H NMR=nuclear magnetic resonance (hydrogen)
Ms=methanesulfonyl
NMP=N-methylpyrollidone
Ns=naphthalene sulphonyl
Pd(OAc)₂=palladium acetate
TBDMS=t-butyldimethylsilyl
TBDPS t-butyldiphenylsilyl
Tf=trifluoromethanesulfonyl
TMS=trimethylsilyl
Ts (tosyl)=p-toluenesulfonyl

The expression “proceed to a sufficient extent” as used herein with reference to the reactions or process steps disclosed herein means that the reactions or process steps proceed to an extent that conversion of the starting material or substrate to product is maximized. Conversion may be maximized when greater than about 5, 10, 15, 20, 25, 30, 35, 40, 45, 50, 55, 60, 65, 70, 75, 80, 85, 90, 95 or 100% of the starting material or substrate is converted to product. These values can be used to define a range, such as 60% to about 90%.

As used herein unless otherwise noted, the term “isotopic label” when describing the substitution of an atom on a substituent group means replacing specific atoms by their isotope. For example, wherein the atom substituted with an isotopic label is carbon, the isotopic label is a carbon atom such as ¹³C; wherein the atom substituted with an isotopic label is hydrogen, the isotopic label is either deuterium or tritium. Preferably, in the compounds of the present disclosure one or more hydrogen atoms are replaced with deuterium.

As used herein, the term “about” means that the numerical value is approximate and small variations would not significantly affect the practice of the disclosed embodiments. Where a numerical limitation is used, unless indicated otherwise by the context, “about” means the numerical value can vary by +10% and remain within the scope of the disclosed embodiments.

The present disclosure is directed to a process for the preparation of compounds of Formula I, as described in Scheme A, below.

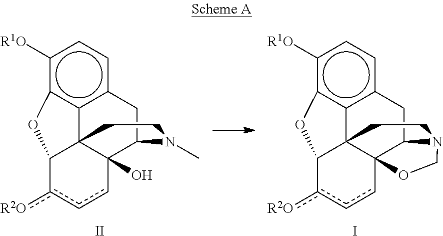

Accordingly, a suitably substituted compound of Formula II, a known compound or compound prepared by known methods; is reacted with a suitably selected oxidizing agent;

wherein the oxidizing agent is selected from the group consisting of oxygen gas or a peroxide; wherein the peroxide is for example t-butylperoxide, and the like; preferably, the oxidizing agent is oxygen; wherein the oxidizing agent is oxygen, then the oxygen is provided into the reaction as a gas, preferably at a pressure in the range of from about 1 bar to about 100 bar, more preferably at a pressure in the range of from about 1 bar to about 20 bar, more preferably at a pressure in the range of from about 5 bar to about 15 bar; and wherein the oxidizing agent is a peroxide such as t-butylperoxide, then the oxidizing agent (peroxide) is present in an amount in the range of from about 1 to about 10 molar equivalents (relative to the moles of the compound of Formula II), preferably in an amount in the range of from about 1 to about 3 molar equivalents;

in the presence of a suitably selected metal catalyst such as platinum, palladium, ruthenium, iron, tungsten, vanadium, iridium, copper and gold, preferably the metal catalyst is a palladium or platinum catalyst, preferably palladium; wherein the metal catalyst is preferably present in an amount in the range of from about 0.001 to about 1 molar equivalents (relative to the moles of the compound of Formula II); preferably in an amount in the range of from about 0.01 to about 0.05 molar equivalents;

in a suitably selected solvent of mixture of solvents such as dimethylacetamide (DMA), dimethylformamide (DMF), dimethylsulfoxide (DMSO), and the like; preferably in dimethylacetamide (DMA);

preferably at a temperature in the range of from about 50° C. to about 150° C., more preferably at a temperature in the range of from about 120° C. to about 140° C.; to yield the corresponding compound of Formula I.

In an embodiment of the present disclosure, the reaction time for conversion of a compound of Formula II to the corresponding compound of Formula I is from about 0.1 hours to about 48 hours, or about 2 hours to about 10 hours in a batch reaction or from about 5 minutes to about 120 minutes, or about 10 minutes to about 390 minutes.

In another embodiment of the present disclosure, the reaction of the compound of Formula II to yield the corresponding compound of Formula I is run in a continuous flow manner (e.g. in a continuous flow reactor).

In an embodiment, the present disclosure is directed to the reaction of 14-hydroxymorphinone (a compound of Formula II), with oxygen in the presence of a metal catalyst to yield the corresponding (oxazolidine-containing) compound of Formula I.

In another embodiment, the present disclosure is directed to the reaction of oxymorphone (a compound of Formula II), with oxygen in the presence of a metal catalyst to yield the corresponding (oxazolidine-containing) compound of Formula I.

Preferably, the compound of Formula I is obtained in a total yield in the range of from about 50% to about 99%, or any amount or range therein, more preferably in a yield in the range of from about 75% to about 99%, more preferably in a yield in the range of from about 85% to about 99%, more preferably in a yield in the range of from about 90% to about 99%, more preferably in a yield of at least about 95%. Preferably, the compound of Formula I is obtained with a purity (as measured by HPLC or NMR) in the range of from about 80% to about 99%, or any amount or range therein, more preferably in a purity in the range of from about 85% to about 99%, more preferably in a purity in the range of from about 90% to about 99%, %, more preferably in a purity in the range of from about 95% to about 99%, more preferably in a purity of at least about 95%.

The metal catalyst is any suitable metal catalyst. In an embodiment, the metal catalyst is a transition metal catalyst. Examples of complexes/compounds which can be used as the metal catalyst include, but are not limited to, catalysts comprising palladium, platinum (e.g. PtCl₂ and K₂PtCl₄), ruthenium (e.g. Ru/C, RuCl₃×H₂O, RuCl₂(PPh₃)₃, RuO₂, and tetrapropylammonium perruthenates, iron (e.g. FeCl₂, FeSO₄, and iron carbonyls such as Fe₂(CO)₉, tungsten (e.g. Na₂WO₄), vanadium (e.g. VO(acac)₂) iridium, copper, gold, and silver complexes. In another embodiment, the metal catalyst is a Pd(0) or a Pd(II) catalyst, for example, but not limited to Pd(OAc)₂, Pd black on palladium-perovskites, or a Pd(0) or a Pd(II) catalyst on any type of solid support (e.g. charcoal, sulfates, carbonates, alumina) or in encapsulated form.

In another embodiment, the metal catalyst is an organo-metallic complex containing organic ligands. The metal catalyst can be chemically bound (e.g., covalently attached) to an inorganic support. For example, the catalyst can be SiliaCat® DPP-Pd (shown below).

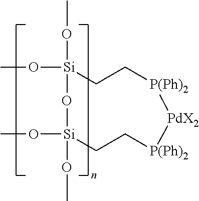

The process of the present disclosure can further include reacting a metal catalyst in an oxidized state to form a reduced state metal catalyst (e.g., a metal catalyst having a zero oxidative state, such as Pd⁰ or Pt⁰). The reaction can take place before, during or after the introduction to the compound containing a tertiary N-methylamine, the introduction to a continuous flow reactor system, or both. With the continuous flow system, the system can include an on-line or in-line preparation section or segment (e.g., heating coil) to incorporate these introduction steps into the reactor system. The reaction can include heating the resultant mixture for a predetermined time, adding one or more additives or stabilizers to the liquid mixture or combinations thereof.

The process of the present disclosure can also include preparing a liquid mixture of the compound containing a tertiary N-methylamine and a metal catalyst (e.g., a palladium or platinum catalyst) wherein the metal catalyst can be in an oxidized state or in reduced state capable of being oxidized. The process further includes reacting the catalyst in an oxidized state to form a reduced state metal catalyst (e.g., a metal catalyst having a zero oxidative state, such as Pd⁰ or Pt⁰). The preparation and reaction of the liquid mixture can occur before, during or after the introduction to the compound containing a tertiary N-methylamine, the introduction to a continuous flow reactor system, or both. With the continuous flow system, the system can include an on-line or in-line preparation section or segment (e.g., heating coil) to incorporate these steps into the reactor system. The reaction can include heating the liquid mixture for a predetermined time, adding one or more additives or stabilizers to the liquid mixture or combinations thereof.

The mixture (e.g. resultant, liquid, reaction) containing the catalyst can be heated to about 70° C., 80° C., 90° C., 100° C., 110° C., 120° C., 130° C., 140° C., 150° C., 160° C., 170° C., 180° C., 190° C., or about 200° C. These values can also be used to define a range, such as about 120° C. to about 140° C. The temperature can also be a few degrees (e.g., 1-10 degrees) below the degradation temperature of the tertiary N-methylamine compound. The temperature can also be the temperature or a few degrees (e.g., 1-10 degrees) above the temperature at which the catalyst forms Pd⁰ or Pt⁰. The mixture containing the catalyst can be heated for about 0.5 min, 1, 2, 3, 4, 5, 6, 7, 8, 9, 10, 30 or about 60 minutes. These values can also be used to define a range, such as about 2 to about 10 minutes. The predetermined time can be dependent on the temperature and other conditions, and can be a time at which a substantial amount of the catalyst forms Pd⁰ or Pt.

In one embodiment, the present disclosure can include a process wherein the metal catalyst is a palladium or platinum catalyst configured to exist in either a +2 oxidation state or a 0 oxidation state, and wherein the reaction occurring in the presence of an alcohol configured to convert the palladium or platinum catalyst from a +2 oxidation state to a 0 oxidation state. The alcohol can be added to the mixture before, during or after a pre-heating step. The alcohol can be any compound that converts the catalyst from Pd² or Pt² to Pd⁰ or Pt⁰. The alcohol can be C₁₋₁₀ primary or secondary alcohol. In one embodiment, the alcohol is ethylene glycol. In another embodiment, the alcohol is 2-propanol. The alcohol can be present in the mixture (e.g. resultant, liquid, reaction) in about 1 equivalent, 2, 3, 4, 5, 6, 7, 8, 9 or about 10 equivalents of the compound containing a tertiary N-methylamine. These values can also be used to define a range, such as about 1 to about 4 equivalents.

In an embodiment of the present disclosure, R¹ and R² are each independently selected from the group consisting of C₁₋₆alkyl, phenyl, naphthyl, indanyl, C₃₋₆cycloalkyl, C₁₋₆alkyleneC₆₋₁₀aryl, C₁₋₆alkyleneC₃₋₆cycloalkyl and an oxygen protecting group. In another embodiment of the present disclosure, R¹ and R² are each independently selected from the group consisting of methyl, ethyl, phenyl, cyclobutyl, cyclopentyl, cyclohexyl, benzyl, cyclobutylmethyl, cyclopentylmethyl, cyclohexylmethyl and an oxygen protecting group. In another embodiment of the present disclosure, each oxygen protecting group is selected from the group consisting of C₁₋₄alkyl acetate, preferably each oxygen protecting group is acetyl.

In another embodiment of the present disclosure, the compound of Formula II is selected from the group consisting of the compound of Formula II(a)

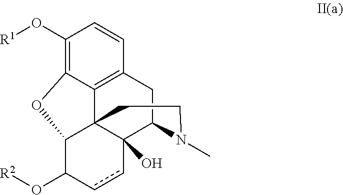

the Formula II(b)

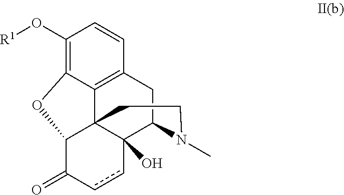

and Formula II(c):

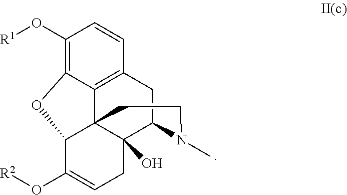

In another embodiment of the present disclosure, the compound of Formula I is selected from the group consisting of the compound of the Formula I(a)

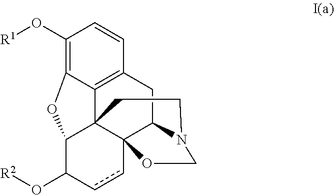

the compound of Formula I(b)

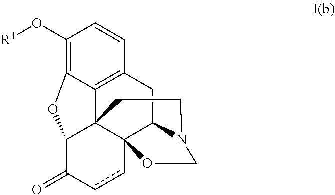

and the compound of Formula I(c)

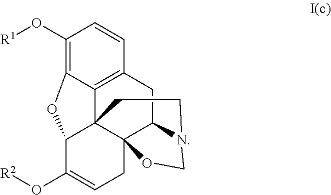

The compounds of Formula I are useful as intermediates for the preparation of a variety of different morphine analogs or derivatives. In an embodiment, the present disclosure is directed to processes for the preparation of said morphine analogs and derivatives, comprising the step of oxidizing a suitably substituted compound of Formula II, according to the method as described in Scheme A above; to yield the corresponding compound of Formula I; and then further reacting the compound of Formula I, as described in Schemes B, C and D below; to yield the corresponding (desired) morphine analog or derivative.

In an embodiment, the present disclosure is directed to processes for the preparation of compounds of Formula III and compounds of Formula IV, as described in Scheme B below.

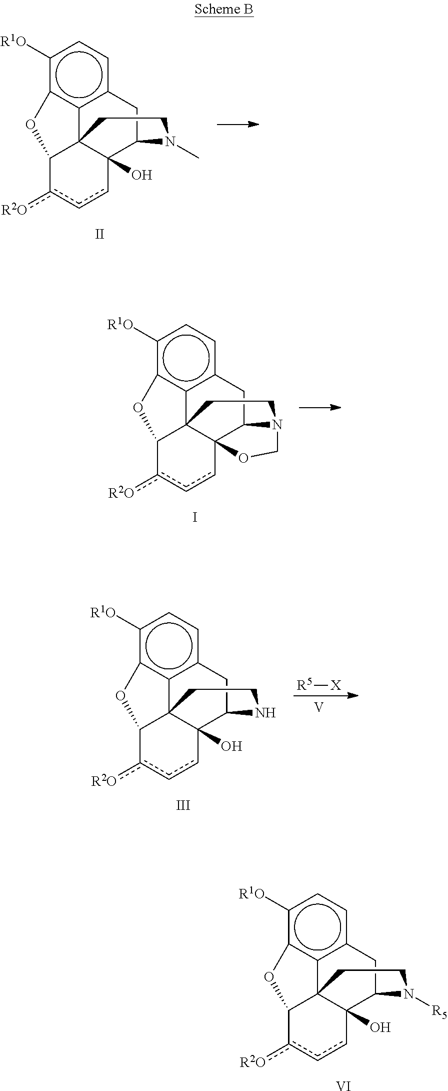

Accordingly, a suitably substituted compound of Formula II, a known compound or compound prepared by known methods; is reacted with a suitably selected oxidizing agent;

wherein the oxidizing agent is selected from the group consisting of oxygen gas or a peroxide; wherein the peroxide is for example t-butylperoxide, and the like; preferably, the oxidizing agent is oxygen; wherein the oxidizing agent is oxygen, then the oxygen is provided into the reaction as a gas, preferably at a pressure in the range of from about 1 bar to about 100 bar, more preferably at a pressure in the range of from about 1 bar to about 20 bar, more preferably at a pressure in the range of from about 5 bar to about 15 bar; and wherein the oxidizing agent is a peroxide such as t-butylperoxide, then the oxidizing agent (peroxide) is present in an amount in the range of from about 1 to about 10 molar equivalents (relative to the moles of the compound of Formula II), preferably in an amount in the range of from about 1 to about 3 molar equivalents;

in the presence of a suitably selected metal catalyst such as platinum, palladium, ruthenium, iron, tungsten, vanadium, iridium, copper and gold, preferably the metal catalyst is a palladium or platinum catalyst, preferably palladium; wherein the metal catalyst is preferably present in an amount in the range of from about 0.001 to about 1 molar equivalents (relative to the moles of the compound of Formula II); preferably in an amount in the range of from about 0.01 to about 0.05 molar equivalents;

in a suitably selected solvent of mixture of solvents such as dimethylacetamide (DMA), dimethylformamide (DMF), dimethylsulfoxide (DMSO), and the like; preferably in dimethylacetamide (DMA); preferably at a temperature in the range of from about 50° C. to about 150° C., more preferably at a temperature in the range of from about 120° C. to about 140° C.; to yield the corresponding compound of Formula I.

The compound of Formula I is hydrolyzed by reacting with a suitably selected acid such as acetic, hydrochloric, sulfuric, and the like, preferably sulfuric; wherein the acid is present in an amount in the range of from about 1 to about 2 molar equivalents (relative to the moles of the compound of Formula I), preferably in an amount in the range of from about 1.25 to about 1.5 molar equivalents;

in the presence of a suitably selected solvent such as dimethylacetamide (DMA), dimethylformamide (DMF), dimethylsulfoxide (DMSO), and the like, preferably dimethylacetamide (DMA); and in the presence of water; wherein the water is present in an amount in the range of from about 1 to about 50 molar equivalents (relative to the moles of the compound of Formula I), preferably in an amount in the range of from about 2 to about 10 molar equivalents;

in a suitably selected solvent such as dimethylacetamide (DMA), dimethylformamide (DMF), dimethylsulfoxide (DMSO), and the like, preferably dimethylacetamide (DMA);

at a temperature in the range of from about 50° C. to about 120° C., preferably at a temperature in the range of from about 60° C. to about 90° C., more preferably at about 80° C.; at a pressure in the range of from about 20 mbar to about 1 bar, preferably at a pressure in the range of from about 100 mbar to about 500 mbar, more preferably at a pressure of about 140 mbar; for one or more periods of between about 5 minutes and about 600 minutes, preferably for one or more periods of between about 5 minutes and about 60 minutes, more preferably for one or more periods of about 10 minutes; to yield the corresponding compound of Formula III.

Alternatively, the compound of Formula I may be hydrolyzed under basic condition; to the corresponding compound of Formula III.

The compound of Formula III is optionally further selectively alkylated by reacting with a suitably substituted compound of Formula V, wherein X is a suitably selected leaving group (counteranion) such as halo, Ms, Ts, Ns, Tf, C₁₋₆acyl, and the like, preferably Br, a known compound or compound prepared by known methods; wherein the compound of Formula V is present in an amount in the range of from about 1 to about 3 molar equivalents (relative to the moles of the compound of Formula III), preferably in an amount in the range of from about 1 to about 1.5 molar equivalents;

in the presence of a suitably selected base such as sodium carbonate, potassium carbonate, dipotassium phosphate, and the like, preferably sodium carbonate; wherein the base is present in an amount in the range of from about 1 to about 3 molar equivalents (relative to the moles of the compound of Formula III), preferably in an amount in the range of from about 1.25 to about 2 molar equivalents;

in a suitably selected solvent such as dimethylacetamide (DMA), dimethylformamide (DMF), dimethylsulfoxide (DMSO), and the like, preferably dimethylformamide (DMF); to yield the corresponding compound of Formula IV.

The present disclosure is further directed to processes for the preparation of compounds of Formula IV and compounds of Formula VI, as described in Scheme C below.

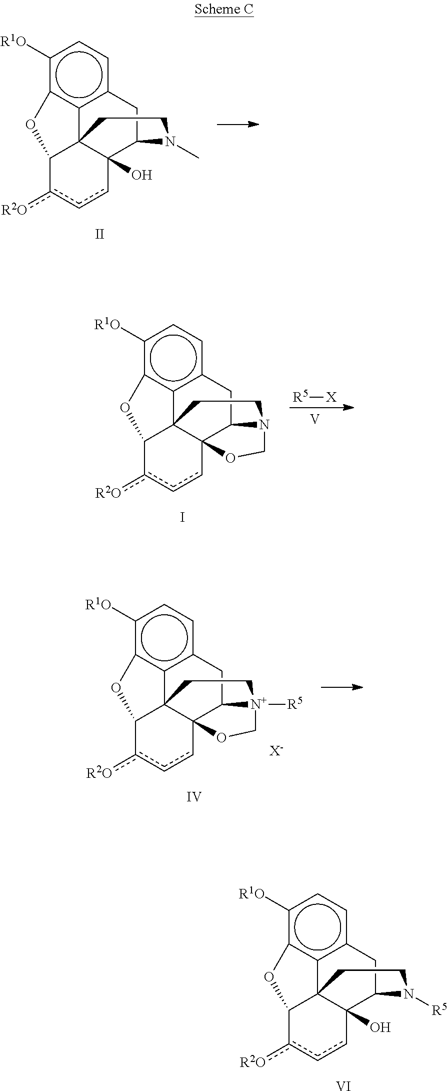

Accordingly, a suitably substituted compound of Formula II, a known compound or compound prepared by known methods; is reacted with a suitably selected oxidizing agent;

wherein the oxidizing agent is selected from the group consisting of oxygen gas or a peroxide; wherein the peroxide is for example t-butylperoxide, and the like; preferably, the oxidizing agent is oxygen; wherein the oxidizing agent is oxygen, then the oxygen is provided into the reaction as a gas, preferably at a pressure in the range of from about 1 bar to about 100 bar, more preferably at a pressure in the range of from about 1 bar to about 20 bar, more preferably at a pressure in the range of from about 5 bar to about 15 bar; and wherein the oxidizing agent is a peroxide such as t-butylperoxide, then the oxidizing agent (peroxide) is present in an amount in the range of from about 1 to about 10 molar equivalents (relative to the moles of the compound of Formula II), preferably in an amount in the range of from about 1 to about 3 molar equivalents;

in the presence of a suitably selected metal catalyst such as platinum, palladium, ruthenium, iron, tungsten, vanadium, iridium, copper and gold, preferably the metal catalyst is a palladium or platinum catalyst, preferably palladium; wherein the metal catalyst is preferably present in an amount in the range of from about 0.001 to about 1 molar equivalents (relative to the moles of the compound of Formula II); preferably in an amount in the range of from about 0.01 to about 0.05 molar equivalents;

in a suitably selected solvent of mixture of solvents such as dimethylacetamide (DMA), dimethylformamide (DMF), dimethylsulfoxide (DMSO), and the like; preferably in dimethylacetamide (DMA); preferably at a temperature in the range of from about 50° C. to about 150° C., more preferably at a temperature in the range of from about 120° C. to about 140° C.; to yield the corresponding compound of Formula I.

The compound of Formula I is selectively alkylated by reacting with a suitably substituted compound of Formula V, wherein X is a suitably selected leaving group (counteranion) such as halo, Ms, Ts, Ns, Tf, C₁₋₆acyl, and the like, and the like, preferably Br, a known compound or compound prepared by known methods; wherein the compound of Formula V is present in an amount in the range of from about 1 to about 3 molar equivalents (relative to the moles of the compound of Formula I), preferably in an amount in the range of from about 1.1 to about 1.5 molar equivalents;

in a suitably selected solvent such as dimethylacetamide (DMA), dimethylformamide (DMF), dimethylsulfoxide (DMSO), and the like, preferably dimethylformamide (DMF); to yield the corresponding compound of Formula IV.

The compound of Formula IV is hydrolyzed by reacting with a suitably selected acid such as acetic, hydrochloric, sulfuric, and the like, preferably sulfuric; wherein the acid is present in an amount in the range of from about 1 to about 5 molar equivalents (relative to the moles of the compound of Formula I), preferably in an amount in the range of from about 2 to about 4 molar equivalents;

in the presence of a suitably selected solvent such as dimethylacetamide (DMA), dimethylformamide (DMF), dimethylsulfoxide (DMSO), and the like, preferably dimethylacetamide (DMA); and in the presence of water; wherein the water is present in an amount in the range of from about 1 to about 50 molar equivalents (relative to the moles of the compound of Formula I), preferably in an amount in the range of from about 2 to about 10 molar equivalents;

in a suitably selected solvent such as water, or a mixture of water with dimethylacetamide (DMA), dimethylformamide (DMF), dimethylsulfoxide (DMSO), and the like, preferably dimethylformamide (DMF);

at a temperature in the range of from about 50° C. to about 120° C., preferably at a temperature in the range of from about 60° C. to about 90° C., more preferably at about 80° C.; at a pressure in the range of from about 20 mbar to about 1 bar, preferably at a pressure in the range of from about 100 mbar to about 500 mbar, more preferably at a pressure of about 140 mbar; for one or more periods of between about 5 minutes and about 600 minutes, preferably for one or more periods of between about 5 minutes and about 60 minutes, more preferably for one or more periods of about 10 minutes; to yield the corresponding compound of Formula VI.

Alternatively, the compound of Formula IV may be hydrolyzed under basic condition; to the corresponding compound of Formula VI.

The present disclosure is further directed to a process for the preparation of compounds of Formula VII, as described in Scheme D, below.

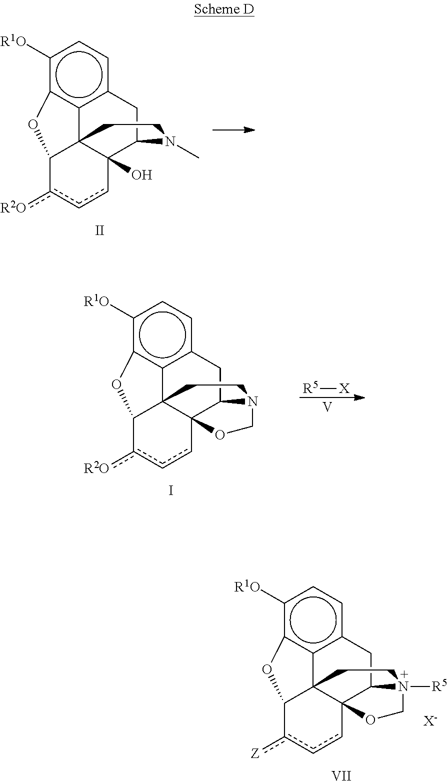

Accordingly, a suitably substituted compound of Formula II, a known compound or compound prepared by known methods; is reacted with a suitably selected oxidizing agent;

wherein the oxidizing agent is selected from the group consisting of oxygen gas or a peroxide; wherein the peroxide is for example t-butylperoxide, and the like; preferably, the oxidizing agent is oxygen; wherein the oxidizing agent is oxygen, then the oxygen is provided into the reaction as a gas, preferably at a pressure in the range of from about 1 bar to about 100 bar, more preferably at a pressure in the range of from about 1 bar to about 20 bar, more preferably at a pressure in the range of from about 5 bar to about 15 bar; and wherein the oxidizing agent is a peroxide such as t-butylperoxide, then the oxidizing agent (peroxide) is present in an amount in the range of from about 1 to about 10 molar equivalents (relative to the moles of the compound of Formula II), preferably in an amount in the range of from about 1 to about 3 molar equivalents;

in the presence of a suitably selected metal catalyst such as platinum, palladium, ruthenium, iron, tungsten, vanadium, iridium, copper and gold, preferably the metal catalyst is a palladium or platinum catalyst, preferably palladium; wherein the metal catalyst is preferably present in an amount in the range of from about 0.001 to about 1 molar equivalents (relative to the moles of the compound of Formula II); preferably in an amount in the range of from about 0.01 to about 0.05 molar equivalents;

in a suitably selected solvent of mixture of solvents such as dimethylacetamide (DMA), dimethylformamide (DMF), dimethylsulfoxide (DMSO), and the like; preferably in dimethylacetamide (DMA); preferably at a temperature in the range of from about 50° C. to about 150° C., more preferably at a temperature in the range of from about 120° C. to about 140° C.; to yield the corresponding compound of Formula I.

The compound of Formula I is reacted with a suitably substituted compound of Formula V, wherein X is a suitably selected leaving group (counteranion) such as halo, Ms, Ts, Ns, Tf, C₁₋₆acyl, and the like, and the like, preferably Br, a known compound or compound prepared by known methods; wherein the compound of Formula V is present in an amount in the range of from about 1 to about 3 molar equivalents (relative to the moles of the compound of Formula I), preferably in an amount in the range of from about 1.1 to about 1.5 molar equivalents;

in a suitably selected solvent such as dimethylacetamide (DMA), dimethylformamide (DMF), dimethylsulfoxide (DMSO), and the like, preferably dimethylformamide (DMF); to yield the corresponding compound of Formula VII.

In certain embodiments of the present disclosure, R¹ and/or R² are each an oxygen protecting group, and said protecting group(s) are protecting groups that can be removed under hydrolysis conditions. For example, when each oxygen protecting group is an alkyl carbonate, hydrolysis under basic conditions results in the removal of the R¹ and/or R² oxygen protecting group simultaneously. In another example, when each oxygen protecting group is an alkyl acetate, hydrolysis under acidic conditions results the removal of the R¹ and/or R² oxygen protecting group simultaneously. A person skilled in the art will appreciate that other protecting groups removable under acidic or basic conditions can also be used.

In certain embodiments of the present disclosure, R¹ and/or R² are oxygen protecting groups, and said protecting group(s) are protecting groups that cannot be removed under conditions the hydrolysis conditions. Said oxygen protecting groups are optionally removed in a separate step after the preparation of the desired compound.

In certain embodiments, the present disclosure is directed to a process for the preparation of a compound selected from the group consisting of compounds of Formula I, compounds of Formula III, compounds of Formula IV, compounds of Formula VI and compounds of Formula VII.

In certain embodiments, the present disclosure is directed to a process for the preparation of a compound selected from the group consisting of compounds of Formula I, compounds of Formula III, compounds of Formula IV, compounds of Formula VI and compounds of Formula VII; wherein the desired product is preferably prepared in an overall yield in the range of from about 50% to about 99%, or any amount or range therein, more preferably in an overall yield in the range of from about 75% to about 99%, more preferably in an overall yield in the range of from about 80% to about 99%, more preferably in an overall yield in the range of from about 90% to about 95%.

In certain embodiments, the present disclosure is directed to a process for the preparation of a compound selected from the group consisting of compounds of Formula I, compounds of Formula III, compounds of Formula IV, compounds of Formula VI and compounds of Formula VII; wherein the desired product is preferably prepared in a purity (as measured for example by HPLC) in the range of from about 80% to about 99%, or any amount or range therein, more preferably in a yield in the range of from about 85% to about 99%, more preferably in a yield in the range of from about 85% to about 95%, more preferably in a yield in the range of from about 90% to about 95%.

In certain embodiments, the present disclosure is directed to a process for the preparation of a compound selected from the group consisting of naltrexone, nalbuphine and naloxone.

In certain embodiments, the present disclosure is directed to a process for the preparation of naltrexone or naloxone, comprising reacting oxymorphone as described herein; wherein the process consists essentially of three reaction steps; and wherein the naltrexone or naloxone product is preferably prepared in an overall yield in the range of from about 50 to about 90%, or any amount or range therein, more preferably in an overall yield in the range of from about 75% to about 90%, more preferably in an overall yield in the range of from about 80% to about 90%.

In some embodiments, and in contrast the processes described in the prior art, the present disclosure relates to a process or processes that does not form an N-oxide compound as an intermediate, final product, or both. An N-oxide compound is a compound containing an N-oxide group, i.e., a R₃N⁺—O⁻ group. The present disclosure also relates to a process or processes that does not form an N-oxide group as part of any intermediate compound, final product, or both. In particular, the process or processes do not form an N-oxide group, such as on the N-17 nitrogen atom of the compounds disclosed herein, e.g., Formula II. In other embodiments, the process or processes include wherein the reaction of a compound disclosed herein, e.g., Formula II, does not include and/or involve the conversion to or the formation of an N-oxide compound or an N-oxide group as part of any intermediate compound, final product, or both.

The processes of the present application may be performed using continuous or batch processes. For commercial scale preparations continuous processes are preferred. Methods of performing chemical processes in continuous or batch modes are known in the art. One skilled in the art will recognize that when continuous processes are used, the reaction temperature and/or pressure may be higher than those used in batch processes; and all such variations and ranges are intended to be included in the processes of the present disclosure.

In some instances, the relatively high reactivity of the α,β-unsaturated carbonyl group of some compounds (e.g., 14-hydroxymorphinone) and their derivatives (e.g., 1,3-oxazolidine), especially during a hydrolysis step, can produce side products. Moreover, although catalyst loading can be reduced in comparison to the batch experiments, additional source(s) can be required to perform hydrogenation. In some embodiments, the 7,8-dehydro group of a compound is initially reduced using in situ generated colloidal Pd(0). Then, the same catalyst can be reused for an oxidative cyclization step. The two steps can be performed separately. The hydrogenation/oxidative cyclization sequence can use two separate heterogeneous Pd catalysts, such as Pd/C for the hydrogenation step and SiliaCat® DPP-Pd in a packed bed reactor for the oxidation heterogeneous. Alternatively, the two steps can be performed in one solution (e.g., “one pot”) with oxygen, for example, as an oxidant.

In another embodiment, the present disclosure is directed to a process for the preparation of compounds of Formula VIII:

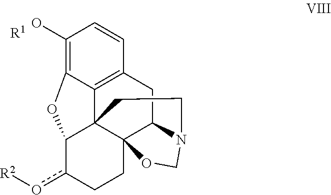

wherein

[symbol] represents a single or double bond; provided that when “ [symbol] ” represents O═, then R² is absent;

R¹ is selected from the group consisting of H, C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;

R² is selected from the group consisting of C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;

wherein one or more hydrogen atoms on the R¹ and R² groups is optionally replaced with a halogen, e.g., F;

including the steps of

providing a compound of formula II;

reacting the compound of formula II in the presence of a first metal catalyst, in a solvent or mixture of solvents, and then

reacting the compound with an oxidizing agent; in the presence of a second metal catalyst, in a solvent or mixture of solvents, to yield the corresponding compound of formula VIII.

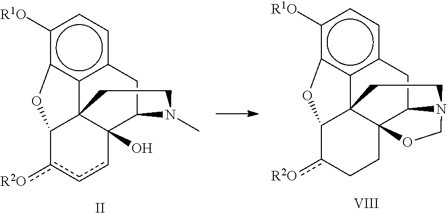

In one embodiment, the compound of formula VIII can be 1,3-oxazolidine.

The first metal catalyst and the second metal catalyst can be the same, or the first metal catalyst and the second metal catalyst can be different. The first metal catalyst, the second metal catalyst, or both can be a colloidal metal catalyst. The first metal catalyst, the second metal catalyst, or both can be an organo-metallic complex containing organic ligands (e.g., SiliaCat® DPP-Pd). The hydrolyzing step, the reaction step or both can also contain an alcohol. The alcohol can be configured to convert an oxidized metal catalyst to an reduced metal catalyst.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

### EXAMPLES

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1

### Batch Aerobic Oxidation of 14-Hydroxycodeinone with Platinum and Palladium Catalysts

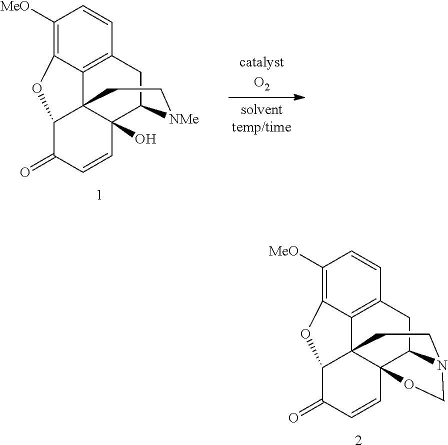

The oxidation of 14-hydroxycodeinone 1 to the oxazolidine-containing compound 2 was performed using a palladium or platinum catalyst with oxygen as oxidant. Batch reactions were performed on a 0.2 mmol scale in N,N-dimethylacetamide (DMA) as solvent under an O₂ atmosphere or under an atmosphere of air in a temperature range of 120 to 140° C. The aerobic oxidation proceeded efficiently with various palladium(0) and platinum(0) species such as colloidal Pd(0), Pd/C or Pt/C, as listed in Table 1, below.

In general, reactions with palladium were more selective than reactions with platinum, even though longer reaction times were required (see Entry 1 and 2 in Table 1). In contrast to reactions with zero-valent palladium and platinum species, reactions with Pd(II) as catalyst proceeded slowly. Heating a mixture of starting material and Pd(OAc)₂ in DMA as solvent resulted in a yellow to orange solution which retained its yellowish appearance throughout the reaction. The aerobic oxidation of 14-hydroxycodeinone 1 to the oxazolidine-containing compound 2 proceeded only slowly in this mixture (see Entry 7 and 8 in Table 1).

Black solutions of finely dispersed palladium(0) nanoparticles were formed in a preceding step by heating a suitable palladium(II) species in the absence of the 14-hydroxycodeinone 1 in DMA as solvent. Specifically, a black solution of colloidal Pd(0) nanoparticles formed upon heating Pd(OAc)₂ in DMA as solvent in the presence of AcOH for few minutes (2 to 20 min) at temperatures of 120 to 140° C. The acetic acid stabilized the colloidal palladium(0) and prevented its agglomeration and precipitation on the vessel walls. The substrate was dissolved in this mixture and the solution was again heated on a hot plate under an oxygen atmosphere or under an atmosphere of air. Conversions of >90% were obtained in these reactions after a reaction time of 30 min using oxygen as oxidant (see Entry 9 in Table 1). The reaction mixture was extracted with CHCl₃/sat NaHCO₃ and the mixture was then concentrated in vacuum. The yield of the product in the isolated (un-purified) mixture was ˜82% (corrected for purity as determined by ¹H-NMR with internal standard).

TABLE 1
Palladium or Platinum catalyzed oxidation of 14-hydroxycodeinone (1) at
120 or 140° C. under an O₂ atmosphere.

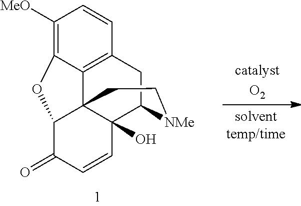 [SMILES: COc1ccc2c3c1O[C@H]1C(=O)C=C[C@@]4(O)[C@@H](C2)N(C)CC[C@]314]

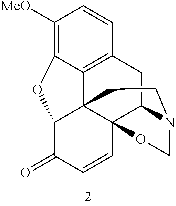 [SMILES: COc1ccc2c3c1O[C@H]1C(=O)C=C[C@]45OCN(CC[C@]314)[C@@H]5C2]

| | | T | time | conv. | sum side |
|---|---|---|---|---|---|
| | cat/(mol %) | [° C.] | [min] | [%] | products [%] |
| 1 | Pd/C (10) | 140 | 30 | 87 | 4 |
| 2 | Pd/C (10) | 140 | 90 | 100 | 6 |
| 3 | Pt/C (10) | 140 | 30 | 100 | 19 |
| 4 | Pt/C (5) | 140 | 30 | 100 | 13 |
| 5 | Pt/C (5) | 140 | 20 | 99 | 13 |
| 6 | Pt/C (5) | 120 | 30 | 76 | 11 |
| 7 | Pd(OAc)₂ (10) | 140 | 30 | 9 | 0 |
| 8 | Pd(OAc)₂ (10) | 140 | 90 | 19 | 0 |
| 9* | Pd(OAc)₂ (10) | 140 | 30 | 100 | 7 |

Conversion was determined as described in Method 1, Example 11; Selectivity was determined as described in Method 2, Example 11. Conditions: 0.2 mmol of substrate, 10 mol % catalyst in 0.6 mL DMA were stirred under an O₂ atmosphere (balloon) on a hot-plate at either 140 or 120° C.

*Pd(OAc)₂ in DMA was heated in the presence of 3.4 equivalents acetic acid prior to the aerobic oxidation.

### Example 2

### Aerobic Oxidation of 14-Hydroxycodeinone with Platinum or Palladium Catalyst in a Continuous Flow Packed Bed Reactor

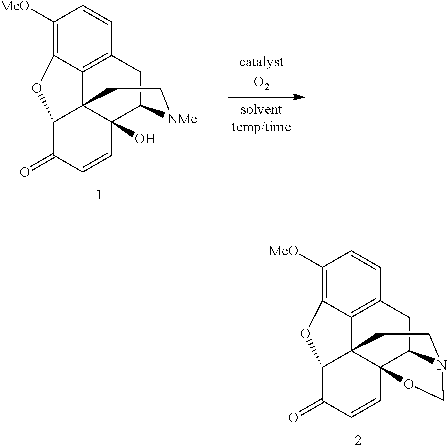

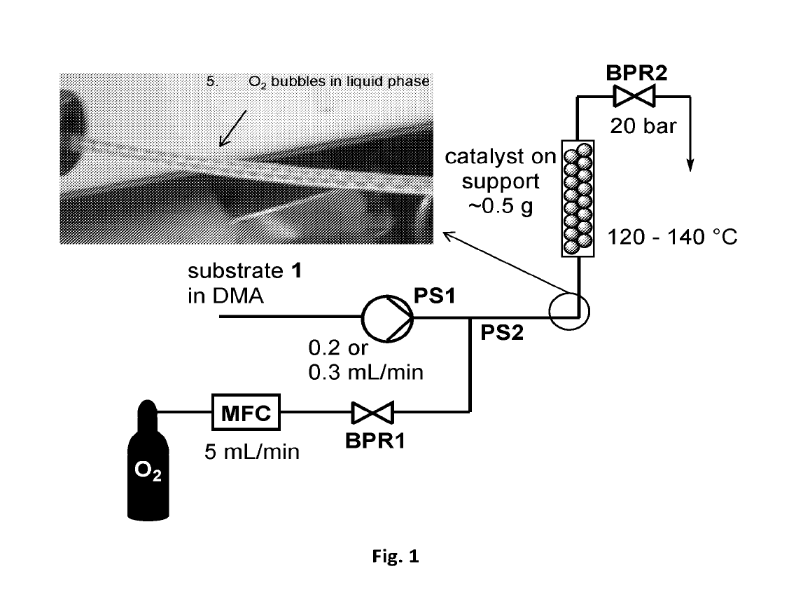

Continuous flow reactions were performed in a packed bed reactor with palladium or platinum catalysts immobilized on solid supports such a charcoal or aluminum oxide, as shown in FIG. 1. The flow reactor consisted of an HPLC pump for pumping the liquid mixture (Uniqsis Pump Module). The pump was connected to a T-mixer (stainless steel) via fluoropolymer tubings ( 1/16″ o.d., 0.8 mm i.d.). Oxygen gas from a gas cylinder (purity 5.0) was introduced to the liquid mixture in the T-mixer. The oxygen gas flow was controlled by a mass flow controller (ThalesNano Gas Module). The T-mixer was connected to a catalyst cartridge via a fluoropolymer tubing ( 1/16″ o.d., 0.8 mm i.d.). The fluoropolymer tubing allowed visually observation of the flow profile. The reaction mixture exited the system through a short cooling loop (stainless steel, 1/16″ o.d., 1 mm i.d.) and a back-pressure regulator (either Swagelok (KCB1H0A2A5P60000) or a static BPR from Upchurch Scientific). A pressure sensor, to determine the system pressure, was integrated in the T-mixer (PS2 in FIG. 1) and a second pressure sensor was integrated directly after the pump (PS1 in FIG. 1).

For the flow experiments, the O₂ and the liquid feed were pumped with flow rates of respectively 5 and 0.3 mL/min. The streams were combined in the T-mixer and the combined stream went through the tubing and then further through the catalyst bed of the bed reactor. The processed reaction mixture left the system through an adjustable back pressure regulator. High single pass conversions of hydroxycodeinone 1 to the oxazolidine-containing compound 2 were obtained in DMA as solvent at a reaction temperature of 140° C. with a catalyst cartridge containing 0.5 grams of the catalyst material (Table 2). Lower conversions were obtained with this bed reactor at a reaction temperature of 120° C. (Entry 4 in Table 2).

TABLE 2
Palladium or Platinum catalyzed oxidation of 14-hydroxycodeinone (1) with oxygen
in a packed bed reactor (500 mg catalyst material (5% catalyst))

| | O₂ | solvent | | T | p | conv. | sum side |
|---|---|---|---|---|---|---|---|
| | (equiv) | (conc [M]) | catalyst | [° C.] | (bar) | [%] | products [%] |
| 1 | 2.3 | DMA (0.32) | Pt/C | 140 | 20.0 | 100 | 14 |
| 2 | 2.3 | DMA (0.32) | Pt/C | 120 | 21.5 | 97 | 7 |
| 3 | 3.3 | toluene/DMF (0.23) | Pt/C | 120 | 20.3 | 87 | 4 |
| 4 | 4.7 | dioxane (0.15) | Pt/C | 120 | 21.0 | 36 | 5 |
| 5 | 2.3 | DMA (0.32) | Pd/Al₂O₃ | 140 | 20.1 | 58 | 4 |
| 6 | 2.3 | DMF (0.32) | Pd/Al₂O₃ | 140 | 18.9 | 39 | 3 |

Conversion was determined as described in Method 1, Example 11; Selectivity was determined as described in Method 2, Example 11. Conditions: flow rate of liquid and O₂ feed: 0.3 and 5 mL/min.

### Example 3

### Aerobic Oxidation of 14-Hydroxycodeinone with Palladium Catalysts in a Continuous Flow Tube Reactor

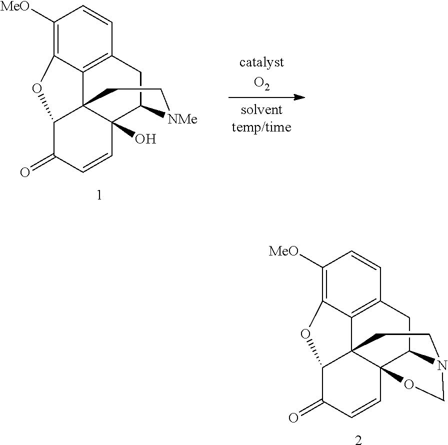

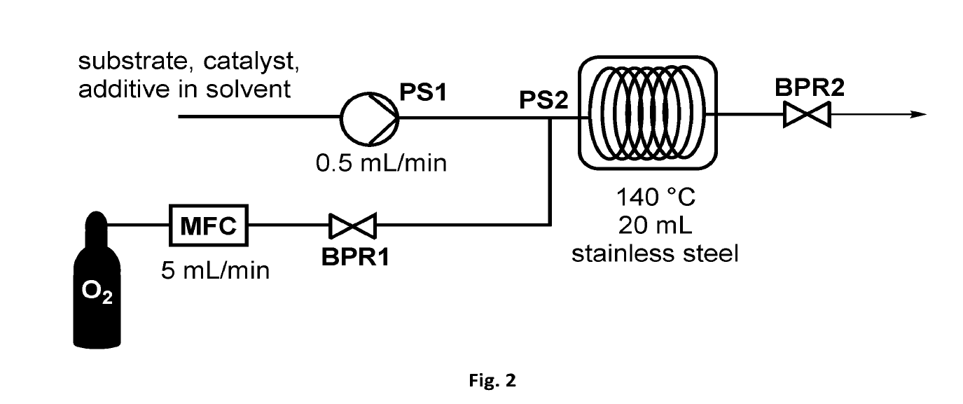

The flow reactor consisted of an HPLC pump for pumping the liquid mixture (Uniqsis Pump Module, as shown in FIG. 2). The pump was connected to a T-mixer (stainless steel) via fluoropolymer tubings ( 1/16″ o.d., 0.8 mm i.d.). Oxygen gas from a gas cylinder (purity 5.0) was introduced to the liquid mixture in the T-mixer. The oxygen gas flow was controlled by a mass flow controller (ThalesNano Gas Module). The T-mixer was connected to a stainless steel tubing ( 1/16″ o.d., 1 mm i.d., 20 mL volume) via a fluoropolymer tubing (stainless steel, 1/16″ o.d., 0.8 mm i.d.). The stainless steel tubing was heated in a GC oven to the desired temperature. The reaction mixture exited the system through a short cooling loop ( 1/16″ o.d., 1 mm i.d.) and a back-pressure regulator (either Swagelok (KCB1H0A2A5P60000) or a static BPR from Upchurch Scientific). A pressure sensor, to determine the system pressure, was integrated in the T-mixer (PS2 in FIG. 2) and a second pressure sensor was integrated directly after the pump (PS1 in FIG. 2).

For continuous flow reactions, 5 mol % Pd(OAc)₂ and AcOH in DMA as solvent were heated for 5 min at 140° C. on a hot plate to generate palladium(0) nanoparticles. The substrate was then dissolved in this solution and the mixture was injected into the flow reactor at a flow rate of 0.5 mL/min. The reaction mixture was mixed with the O₂ feed at a flow rate of 5 mL/min in the T-mixer, and the combined solution went through the stainless steel tube reactor. Good conversions and high selectivities were obtained in these reactions with 2 to 5 equivalents of AcOH after reaction times of 25 min. Lower amounts of AcOH reduced the conversions (Table 3). When the palladium(II) catalyst was directly heated in the presence of the substrate, colloidal palladium(0) particles did not form and a subsequent flow reaction gave only low conversions (Entry 4 in Table 3).

### (5aR,6R,8aS,8a1S,11aR)-2-methoxy-5,5a-dihydro-7H-6,8a1-ethanofuro[2′,3′,4′,5′:4,5]phenanthro[9,8a-d]oxazol-1(11aH)-one (oxazolidine-containing Compound 2)

¹H NMR (300 MHz, CDCl₃) δ 6.75 (d, J=8.3 Hz, 1H), 6.69 (d, J=8.3 Hz, 1H), 6.56 (d, J=10.1 Hz, 1H), 6.23 (d, J=10.1 Hz, 1H), 4.80-4.74 (m, 2H), 4.64 (s, 1H), 3.84 (s, 3H), 3.46-3.36 (m, 2H), 3.18 (dd, J=19.1, 7.9 Hz, 1H), 2.89-2.79 (m, 2H), 2.45-2.30 (m, 1H), 1.61 (dd, J=12.8, 4.0 Hz, 1H).

TABLE 3
Flow oxidation of 14-hydroxycodeinone (1) at
140° C., 5 mol % Pd(OAc)₂ as pre-catalyst

| | AcOH [equiv] | conv. [%] | sum side products [%] |
|---|---|---|---|
| 1 | 1 | 65 | 7 |
| 2 | 2 | 91 | 8 |
| 3 | 3 | 97, 98 | 8, 9 |
| 4* | 3 | 30 | 4 |
| 5 | 5 | 95 | 8 |

Conversion was determined as described in Method 1, Example 11; Selectivity was determined as described in Method 2, Example 11. Condition: 100 mg substrate, AcOH, 5 mol % Pd(OAc)₂ in 1 mL DMA; flow rate liquid/O₂: 0.5/5 mL/min (1.4 equivalents of O₂); 13 to 15 bar back pressure; 22 to 25 min residence time; the reaction mixtures were heated in the absence of the substrate for 5 to 10 min at temperatures of 120 to 140° C. before the continuous flow reaction.

* The reaction mixture was directly injected into the flow reactor without prior generation of palladium(0) particles.

For experiments 1, 2, 3 and 5, Pd(OAc)₂ and 3.4 equivalents of acetic acid in DMA as solvent were heated in the absence of the substrate for 5 min at 140° C., while for experiment 4 the mixture was heated in the presence of the substrate.

### Example 4

### Batch Aerobic Oxidation of 14-Hydroxymorphinone with Platinum and Palladium Catalysts

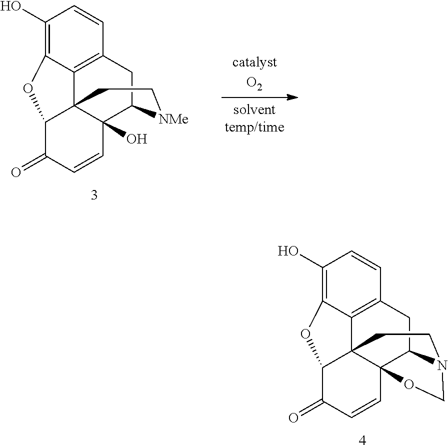

For batch oxidations of 14-hydroxymorphinone 3 to the oxazolidine-containing compound 4, 7.5 mg of Pd(OAc)₂ (10 mol %) and 57 μL of AcOH (3 equiv) were first heated in 1 mL DMA on a hotplate for 5 min at 140° C. to generate finely dispersed palladium(0) particles. 14-Hydroxymorphinone (100 mg) was then dissolved in this solution and the solution was then heated to 140° C. under an O₂ atmosphere. 14-Hydroxymorphinone is not soluble in DMA at room temperature but it was observed to dissolve at temperatures of around 70° C. During the reaction the oxazolidine-containing compound 4 precipitated from the reaction mixture as a brown solid.

Analysis of the reaction mixture revealed that the palladium catalyzed demethylation of 14-hydroxymorphinone 3 was complete after a reaction time of 40 min at 140° C. After the reaction, the mixture was concentrated and toluene was added to precipitate the product. The oxazolidine-containing compound 4 were isolated as a brown solid (90 mg). Reactions with palladium(0) species, such as unsupported zerovalent palladium(0) or palladium(0) on charcoal, gave results comparable to those obtained with colloidal palladium prepared in-situ from Pd(OAc)₂ as pre-catalyst. A fast reaction was observed with platinum(0) species, including platinum on charcoal (Entry 7 and 8 in Table 4).

TABLE 4
Batch oxidation of 14-hydroxymorphinone (3) at 140° C. with
Pd(0) or Pt(0) under an O₂ atmosphere.

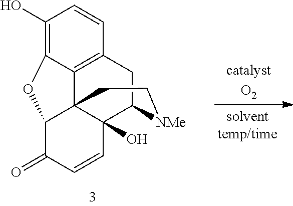 [SMILES: CN1CC[C@]23c4c5ccc(O)c4O[C@H]2C(=O)C=C[C@@]3(O)[C@H]1C5]

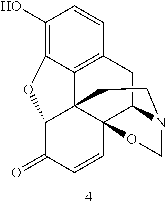 [SMILES: O=C1C=C[C@]23OCN4CC[C@@]25c2c(ccc(O)c2O[C@@H]15)C[C@@H]43]

| | cat(mol %) | solvent | time [min] | conv. [%] |
|---|---|---|---|---|
| 1 | Pd/C(10) | DMA | 40 | 73 |
| 2 | Pd(10) | DMA | 45 | 74 |
| 3 | Pd(10) | DMA | 90 | 88 |
| 4 | Pd(5) | DMA | 90 | 74 |
| 5 | Pd(10) | DMF | 45 | 65 |
| 6 | Pd(10) | DMF | 90 | 80 |
| 7 | Pt/C(5) | DMA | 45 | 98 |
| 8 | Pt/C(2.5) | DMA | 45 | 82 |

Conversion was determined as described in Method 3, Example 11; Conditions: 0.33 mmol of substrate and catalyst in 1.0 mL DMA were stirred under an O₂ atmosphere (balloon) on a hot-plate at 140° C.

The palladium catalyzed oxidation with palladium(0) proceeded well in polar aprotic solvents, including DMA and DMSO. Other solvents, including dioxane, toluene, butanone, EtOAc, MeCN and i-PrOH, yielded slower reactions or no reaction. A reaction with AcOH and AcOH/H₂O gave the free secondary amine, 14-hydroxynormorphinone, as the main product.

Active palladium(0) nanoparticles similar to those formed from Pd(OAc)₂ in DMA as solvent cannot be generated in DMSO as solvent. Heating Pd(OAc)₂ in DMSO as solvent yielded a grayish-black solution of metallic palladium(0), and a subsequent reaction with the hydroxymorphinone dissolved in this solution under an oxygen atmosphere did not give any reaction. No improvement was obtained by addition of AcOH.

Reactions in DMSO as solvent with various palladium(0) species, including unsupported palladium(0) particles or palladium(0) on charcoal, proceeded as well as experiments in DMA with freshly formed colloidal palladium(0) particles. A reaction with 5 mol % Pd/C in DMSO-d6 as solvent revealed a product purity of >90% after a reaction time of 40 min at 140° C. according to ¹H-NMR with internal standard.

From a batch reaction on a 1 g scale in DMSO as solvent with 5 mol % Pd/C, the palladium was recovered after the reaction as a black powder by hot filtration. Water was then added to the filtrate to precipitate the oxazolidine-containing compound 4 (as shown in Table 4 above) in 77% product yield as a brown powder. A corresponding reaction with Pd(OAc)₂/AcOH/DMA on the same scale required 200 min at 140° C. to yield good conversion. Precipitation with water yielded 596 mg of the product (compound 4 as shown in Table 4 above) (60% product yield).

Further optimization reactions revealed that the reaction temperature for the reaction in DMA as solvent can be reduced to 120° C. without reducing the reaction rate significantly (Table 5). Large amounts of product precipitated from the reaction mixture over the course of the reaction. The catalyst loading was reduced to 2.5 mol %, which resulted in >95% conversion after a reaction time of 30 min. Reactions with air instead of oxygen gave slightly reduced reaction rates (˜80 min for complete conversion at 120° C.; Table 5). After a reaction on a 500 mg scale with 5 mol % Pd(OAc)₂ as pre-catalyst, the mixture was cooled and the precipitate was isolated by filtration to yield the product (370 mg) in good purity (as interpreted by the lack of peaks resulting from other species in an ¹H NMR of a sample to the isolated product) (75% yield).

### (5aR,6R,8aS,8a¹S,11aR)-2-hydroxy-5,5a-dihydro-7H-6, 8a¹-ethanofuro[2′,3′,4′,5′:4,5]phenanthro[9,8a-d]oxazol-11(11aH)-one (the oxazolidine-containing Compound 4)

¹H NMR (300 MHz, DMSO) δ 9.28 (s, 1H), 6.79 (d, J=10.1 Hz, 1H), 6.63 (d, J=8.1 Hz, 1H), 6.59 (d, J=8.2 Hz, 1H), 6.18 (d, J=10.1 Hz, 1H), 4.74 (d, J=6.3 Hz, 1H), 4.73 (s, 1H), 4.60 (d, J=5.5 Hz, 1H), 3.47 (d, J=7.4 Hz, 1H), 3.30 (d, J=25.5 Hz, 1H), 3.09 (dd, J=18.9, 7.6 Hz, 1H), 2.82-2.65 (m, 2H), 2.34-2.23 (m, 1H), 1.37 (dd, J=12.7, 3.3 Hz, 1H).

TABLE 5
Batch oxidation of 14-hydroxymorphinone (3) with
Pd(OAc)₂ as pre-catalyst at 120° C. in DMA as solvent.

| | Pd(OAc)₂ [mol %] | oxidant | reaction time [min] | Conv [%] |
|---|---|---|---|---|
| 1 | 5.0 | O₂ | 30 | 98 |
| 2 | 5.0 | O₂ | 120 | 100 |
| 3 | 5.0 | air | 30 | 68 |
| 4 | 5.0 | air | 120 | 98 |
| 5 | 2.5 | O₂ | 30 | 96 |
| 6 | 2.5 | O₂ | 120 | 98 |
| 7 | 2.5 | air | 30 | 60 |
| 8 | 2.5 | air | 120 | 96 |

Conversion was determined as described in Method 4, Example 11; Conditions: 60 mg substrate, 3 equivalents of AcOH, Pd(OAc)₂ in DMA as solvent. Pd(OAc)₂ and the acetic acid in DMA as solvent were heated in the absence of the substrate prior to the oxidation to generate colloidal palladium(0) particles.

Different amounts of acetic acid were explored for the palladium catalyzed oxidation. For these reactions the Pd(OAc)₂ (5 mol %) in DMA and the respective amounts of acetic acid were heated for 10 min at 120° C. The 14-hydroxymorphinone was then dissolved in this solution, and the mixture was heated under an atmosphere of air under good stirring for further 60 min to 120 min at 120° C. The best results were obtained with 1.5 to 3 equivalents of AcOH. Lower amounts of AcOH decreased the reaction rate, while larger amounts of AcOH decreased both reaction rate and reaction purity. Large amounts of precipitate were formed with 1.5 to 3 equivalents of AcOH (Table 6).

TABLE 6
Batch oxidation of 14-hydroxymorphinone (3) with different
amounts of AcOH with atmospheric oxygen.

| | AcOH [equiv] | time [min] | conv. [%] | oxazolidine [%] |
|---|---|---|---|---|
| 1 | 0.0 | 60 | 33 | 31 |
| 2 | 0.75 | 60 | 34 | 33 |
| 3 | 1.5 | 60 | 98 | 93 |
| 4 | 3 | 60 | 96 | 91 |
| 5 | 3 | 120 | 99 | 93 |
| 6 | 6 | 60 | 33 | 30 |
| 7 | 6 | 120 | 49 | 45 |
| 8 | 12 | 60 | 16 | 13 |
| 9 | 12 | 120 | 27 | 16 |

Conversion was determined as described in Method 3, Example 11; Conditions: 60 mg of substrate (0.2 mmol), 5 mol % of Pd(OAc)₂ and 0.6 mL of DMA. Reaction temperature 120° C.; Pd(OAc)₂ and the acetic acid in DMA as solvent were heated in the absence of the substrate prior to the oxidation to generate colloidal palladium(0) particles.

The reaction rate depended strongly on the source and batch of the Pd(OAc)₂. With the Pd(OAc)₂ used for the experiments described above, the reaction proceeded as expected (Entry 2 in Table 7). Pd(OAc)₂ from Tokyo Chemical Industry Co., Ltd. (TCI) or other batches of Pd(OAc)₂ from Aldrich gave slower reactions (Table 7).

TABLE 7
Batch oxidation of 14-hydroxymorphinone (3)
at 140° C. with 10 mol % Pd(OAc)₂ and 3 equivalents
of AcOH under an O₂ atmosphere.

| | catalyst (source) | time [min] | conv. [%] |
|---|---|---|---|
| 1 | Pd(OAc)₂ (TCI) | 40 | 20 |
| 2 | Pd(OAc)₂ | 40 | 89 |
| | (Aldrich-MKBN8465V) | | |
| 3 | Pd(OAc)₂ | 40 | 54 |
| | (Aldrich-MKBS7764V) | | |
| 4 | Pd(OAc)₂ | 40 | 76 |
| | (Aldrich-MKBL7649V) | | |

Conversion was determined as described in Method 3, Example 11; Conditions: 0.33 mmol of substrate, 3 equivalent of AcOH and 10 mol % Pd(OAc)₂ in 1.0 mL DMA were stirred under an O₂ atmosphere (balloon) on a hot-plate at 140° C. All reaction mixtures were heated prior to the reaction in the absence of the substrate for 5 min at 140° C. to generate palladium(0) particles from Pd(OAc)₂.

### Example 5

### Aerobic Oxidation of 14-Hydroxymorphinone with Palladium Catalysts in a Continuous Flow Tube Reactor

Continuous flow reactions were performed in a reactor setup as described in FIG. 2 (Example 3). For continuous flow reactions, 5 mol % Pd(OAc)₂ and AcOH in DMA as solvent were heated for 5 min at 140° C. on a hot plate to generate palladium(0) nanoparticles. The substrate was then dissolved in this solution and the mixture was injected into the flow reactor at a flow rate of 0.5 mL/min. The reaction mixture was mixed with the O₂ feed at a flow rate of 5 mL/min in a T-mixer, and the combined solution went through the 20 mL stainless steel tube reactor (1 mm inner diameter). A reaction on a 100 mg scale in DMA as solvent yielded a conversion of 84% after a residence time of ˜22 min (Entry 1 in Table 8). Subsequent reactions on a 200 and 300 mg scale provided conversions of 94% (Entry 2 and 3 in Table 8). A reaction on a 1 g scale yielded the product with >95% conversion (Entry 4 in Table 8).

A conversion of 80% was obtained after a residence time of ˜25 min at a reaction temperature of 120° C. with 2.5 mol % Pd(OAc)₂. A reaction with 5 mol % Pd(OAc)₂ gave 94% conversion (Table 8). Even though product precipitated from the mixture, the reactor did not clog for reactions on this scale, and the processed mixture left the reactor as a slurry.

TABLE 8
Flow oxidation of 14-hydroxymorphinone (3) with
Pd(OAc)₂ at 120 to 140° C. in DMA as solvent.

| | scale [mg] | Pd(OAc)₂ [mol %] | Temp. [° C.] | conv. [%] |
|---|---|---|---|---|
| 1 | 100 | 5 | 140 | 84 |
| 2 | 200 | 5 | 140 | 94 |
| 3 | 300 | 5 | 140 | 94 |
| 4 | 1000 | 5 | 140 | >95 |
| 5 | 200 | 2.5 | 120 | 80 |
| 6 | 500 | 5.0 | 120 | 94 |

Conversion was determined as described in Method 4, Example 11; Condition: Substrate, 3 equivalents AcOH, Pd(OAc)₂ in DMA; flow rate liquid/O₂: 0.5/5 mL/min (1.3 equivalents of O₂); 13 to 15 bar back pressure; 22 to 25 min residence time; the reaction mixtures were heated in the absence of the substrate for 5 to 10 min at temperatures of 120 to 140° C. before the continuous flow reaction.

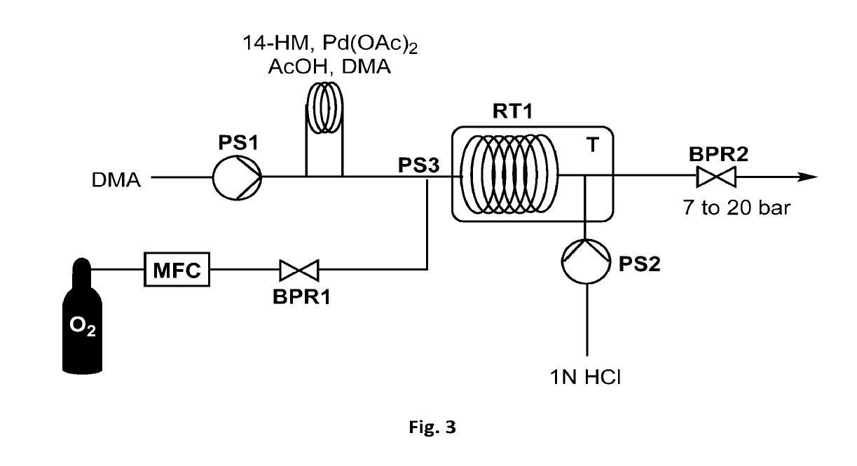

To reduce the risk to clog the reactor, the stainless steel tube reactor was replaced by a thick walled tubing made of a gastight fluoropolymer. The inner diameter of the tubing was 1.6 mm. The total residence volumes of the tested tubings were 10 to 57 mL. To get the reaction mixture through the back pressure regulator as a homogeneous solution, 1N HCl was fed into the reactor before the back pressure regulator (FIG. 3). The precipitate quickly dissolved upon mixing with aqueous HCl. The first experiments were performed with a reactor of 10 mL residence volume. Conversion of 81-97% and good selectivities of 80-96% were obtained after a residence time of only 9 to 10 min at 120° C. with 1.25 mol % Pd(OAc)₂ as pre-catalyst. The selectivity for this reaction was higher than that usually observed in batch. Decreasing the concentration of the mixture reduced the reaction rate, without affecting reaction selectivity (Table 9).

TABLE 9
Flow oxidation of 14-hydroxymorphinone (3) with
Pd(OAc)₂ at 120° C. in DMA as solvent.

| | scale | Weight | O₂ | Res. time | conv. | oxazolidine |
|---|---|---|---|---|---|---|
| | [mg] | percent [%] | [equiv] | [min] | [%] | [%] |
| 1 | 300 | 11 | 1.3 | 9.5 | 97% | 96% |
| 2 | 300 | 7 | 2 | 10.5 | 90% | 89% |
| 3 | 300 | 5 | 2.7 | 9.5 | 81% | 80% |
| 4 | 600 | 11 | 1.3 | 11 | 89% | 83% |

Conversion was determined as described in Method 5, Example 11; Conditions: Substrate, AcOH (3 equiv), DMA and 1.25 mol % Pd(OAc)₂; flow rate gaseous and liquid phase: 5 and 0.5 mL/min; back pressure: 9 bar; the reaction mixtures were heated in the absence of the substrate for 10 min at a temperature of 140° C. before the continuous flow reaction.

Further reactions were performed in a reactor of 57 mL residence volume. The reactions proceeded with selectivity for the desired product (as noted in the Table 9 above), but full conversion was generally not achieved. Increasing the residence time by decreasing the flow rate did not increase the conversion, while increasing the reaction temperature decreased the conversion. Changing the equivalents of O₂ did not have any appreciable effect. Increasing the amounts of Pd(OAc)₂ did not increase the conversion, but decreased the purity of the reaction (Table 10).

TABLE 10
Flow oxidation of 14-hydroxymorphinone (3) with Pd(OAc)₂
at various temperatures in DMA as solvent.

| | scale | Wt. % | Pd(OAc)₂ | flow rate | temp | O₂ | res time | conv. | oxazo- | isol |
|---|---|---|---|---|---|---|---|---|---|---|
| | [mg] | [%] | [mol %] | O₂/liquid | [° C.] | [equiv] | [min] | [%] | lidine [%] | [mg]* |
| 1 | 600 | 7 | 1.25 | 20/2 | 120 | 2 | 15 | 94% | 93% | 436 |
| 2 | 600 | 7 | 1.25 | 15/1.5 | 120 | 2 | 21 | 90% | 82% | 411 |
| 3 | 600 | 7 | 1.25 | 15/1.5 | 130 | 2 | 19 | 84% | 81% | 309 |
| 4 | 600 | 11 | 1.25 | 20/2 | 120 | 1.3 | 13 | 91% | 85% | 407 |
| 5 | 600 | 11 | 1.25 | 15/2 | 120 | 1 | 19 | 92% | 88% | 408 |
| 6 | 600 | 11 | 1.25 | 25/2.5 | 120 | 1.3 | 11 | 86% | 81% | 381 |
| 7 | 600 | 11 | 1.5 | 20/2 | 120 | 1.3 | 13 | 92% | 85% | 372 |
| 8 | 600 | 11 | 2 | 20/2 | 120 | 1.3 | 13 | 95% | 81% | — |
| 9 | 600 | 11 | 1.5 | 30/2 | 120 | 2 | 11 | 91% | 83% | 379 |
| 10 | 1200 | 11 | 1.5 | 20/2 | 120 | 1.3 | 15 | 92% | 82% | 785 |

Conversion was determined as described in Method 5, Example 11; Conditions: substrate, AcOH (3 equiv), DMA and Pd(OAc)₂; back pressure between 9 to 11 bar; the reaction mixtures were heated in the absence of the substrate for 10 min at a temperature of 140° C. before the continuous flow reaction.

* isolated material after hydrolysis and precipitation with ammonia.

Varying the conditions for the activation of the palladium did not have any appreciable effect on the subsequent oxidation reaction (Table 11).

TABLE 11
Flow oxidation of 14-hydroxymorphinone (3) under
varying conditions for the palladium activation.

| | | res time | | | |
|---|---|---|---|---|---|
| | activation | [min] | conv. [%] | oxazolidine [%] | isol [mg]* |
| 1 | 20/120 | 13 | 91% | 85% | 436 |
| 2 | 20/140 | 13 | 93% | 85% | 423 |
| 3 | 10/160 | 13 | 93% | 87% | — |

Conversion was determined as described in Method 5, Example 11; Conditions: 600 mg of substrate, AcOH (3 equiv), 6 mL of DMA and 1.25 mol % of Pd(OAc)₂; 120° C. reaction temperature; flow rate gaseous and liquid feed: 20 and 2 mL/min (1.3 equiv of O₂); back pressure between 9 to 11 bar; the reaction mixtures were heated in the absence of the substrate for the indicated time at the indicated temperature before the continuous flow reaction.

* isolated material after hydrolysis.

Conversions of ˜96% were obtained with back pressures <9 bar (Table 12). A reaction with the coil reactor immersed in an ultrasonic bath yielded results comparable to those obtained under standard conditions.

TABLE 12
Flow oxidation of 14-hydroxymorphinone (3)
at various back pressures.

| | flow rate | O₂ | pressure | res time | conv. | oxazolidine |
|---|---|---|---|---|---|---|
| | O₂/liquid | [equiv] | [bar] | [min] | [%] | [%] |
| 1 | 10/1 | 1.3 | 8 | 27 | 96% | 86% |
| 2 | 10/1 | 1.3 | 7 | 24 | 96% | 90% |
| 3 | 10/1 | 1.3 | 6.5 | 21 | 96% | 90% |
| 4 | 10/1 | 1.3 | 6 | 19 | 94% | 88% |

Conversion was determined as described in Method 5, Example 11; Conditions: 600 mg of substrate, AcOH (3 equiv), 6 mL of DMA and 1.25 mol % of Pd(OAc)₂; 120° C. reaction temperature; flow rate gaseous/liquid phase 10/1 mL/min (1.3 equiv of O₂); the reaction mixtures were heated in the absence of the substrate for 10 to 20 min at a temperature of 140° C. before the continuous flow reaction.

### Example 6

### Synthesis of 3-Acetyl-14-hydroxymorphinone 5

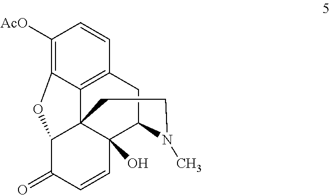 [SMILES: CC(=O)Oc1ccc2c3c1O[C@H]1C(=O)C=C[C@@]4(O)[C@@H](C2)N(C)CC[C@]314]

3-Acetyl-14-hydroxymorphinone 5 was synthesized according to literature procedures (WERNER, L., et al., Adv. Synth. Catal., 2012, pp. 2706-2712, Vol. 354) reacting 1 equiv of Ac₂O, 1 equiv of K₂CO₃ and in THF as solvent. The reaction was selective for the 3-acetyl-14-hydroxymorphinone 5 and only small amounts of the 3,14-diacetyl derivative were formed (<1%). 6.65 gram of a white solid were isolated from a reaction with 6 gram of 14-hydroxymorphinone after removing the salts by filtration and evaporation of the solvent (97% yield).

### Example 7

### Aerobic Oxidation of 3-Acetyl-14-Hydroxymorphinone with Palladium Catalysts in a Continuous Flow Tube Reactor

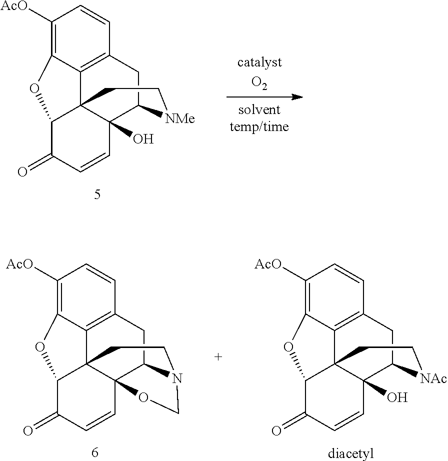

Continuous flow oxidations were performed in the same setup as shown in FIG. 3, but without the HCl quench feed. For continuous flow reactions, the respective amounts of Pd(OAc)₂ and AcOH in DMA as solvent were heated for 5 to 20 min at 140° C. on a hot plate to generate palladium(0) nanoparticles. The substrate was then dissolved in this solution and the mixture was injected into the flow reactor at the flow rates indicated in Table 13. The reaction mixture was mixed with the O₂ feed in a T-mixer, and the combined solution went through the 57 mL tube reactor. >90% conversion was obtained after a residence time of ˜22 min with 1.25 mol % catalyst. Increasing the residence time by decreasing the flow rate did not increase the conversion. Increasing the catalyst loading, decreasing the pressure or the equivalents of O₂ did not have any effect (Table 13). The main side-product in these reactions was the 3,17-diacetyl-14-hydroxynormorphinone.

TABLE 13
Flow oxidation of 3-acetyl-14-hydroxymorphinone (5) with oxygen.

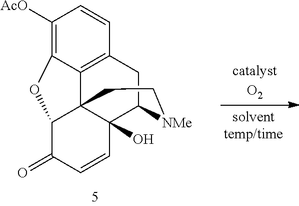 [SMILES: CC(=O)Oc1ccc2c3c1O[C@H]1C(=O)C=C[C@@]4(O)[C@@H](C2)N(C)CC[C@]314]

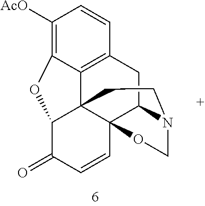 [SMILES: CC(=O)Oc1ccc2c3c1O[C@H]1C(=O)C=C[C@]45OCN(CC[C@]314)[C@@H]5C2]

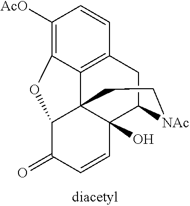

| | | flow rate | | | res | | selec- | |
|---|---|---|---|---|---|---|---|---|
| | Pd(OAc)₂ | O₂/liquid | temp | p | time | conv. | tivity | diacetyl |
| | [mol %[ | [mL/min] | [° C.] | [bar] | [min] | [%] | [%] | [%] |
| 1 | 1.25 | 10/1 | 120 | 7 | 22 | 93 | 97.2 | 1.2 |
| 2 | 1.25 | 5/0.5 | 120 | 7 | 52 | 92 | 95.3 | 1.5 |
| 3 | 2.0 | 10/1 | 120 | 7 | 23 | 91 | 95.6 | 2.0 |
| 4 | 2.0 | 10/1 | 110 | 7 | 23 | 88 | 96.8 | 1.2 |
| 5 | 1.25 | 5/0.5 | 120 | 3.5 | 28 | 91 | 96.2 | 1.9 |
| 6 | 2.5 | 5/0.5 | 120 | 3.5 | 28 | 94 | 94.5 | 2.8 |
| 7 | 1.25 | 5/0.7 | 120 | 3.5 | 28 | 89 | 95.9 | 1.8 |

Conversion was determined as described in Method 5, Example 11. Selectivity was determined as described in Method 2, Example 11. Conditions: Substrate (600 mg), AcOH (3 equiv), DMA (6 mL) and Pd(OAc)₂; the reaction mixtures were heated in the absence of the substrate for 10 to 20 min at a temperature of 140° C. before the continuous flow reaction.

Optimization of the amount of acetic acid was performed at different pressures and with different catalyst loadings. The best results were generally obtained with around 6 equivalents of acetic acid (Table 14). Acetic acid amounts below 3 equivalents reduced the conversion significantly (Entry 1 and 4 in Table 14). Low conversions were obtained when Pd(OAc)₂, acetic acid and substrate in DMA as solvent was directly injected into the flow reactor, without prior formation of palladium(0) particles (Entry 14 in Table 14).

TABLE 14
Flow oxidation of 3-acetyl-14-hydroxymorphinone (5) with oxygen.

| | | acetic | flow rate | | | Res | | | |
|---|---|---|---|---|---|---|---|---|---|
| | Pd(OAc)₂ | acid | O₂/liquid | temp | press | time | conv. | selectivity | diacetyl |
| | [mol %] | [equiv] | [mL/min] | [° C.] | [bar] | [min] | [%] | [%] | [%] |
| 1 | 1.25 | 1.5 | 20/2 | 120 | 8 | 30 | 45 | 96.4 | 1.3 |
| 2 | 1.25 | 3 | 20/2 | 120 | 8 | 27 | 70 | 95.9 | 1.5 |
| 3 | 1.25 | 6 | 20/2 | 120 | 8 | 30 | 82 | 94.5 | 2.5 |
| 4 | 1.25 | 1.5 | 5/0.5 | 120 | 4 | 33 | 51 | 96.6 | 1.3 |
| 5 | 1.25 | 3 | 5/0.5 | 120 | 4 | 32 | 71 | 96.3 | 1.6 |
| 6 | 1.25 | 4 | 5/0.5 | 120 | 4 | 34 | 76 | 95.3 | 2.1 |
| 7 | 1.25 | 6 | 5/0.5 | 120 | 4 | 33 | 81 | 95.3 | 2.0 |
| 8 | 1.25 | 8 | 5/0.5 | 120 | 4 | 32 | 86 | 94.8 | 2.8 |
| 9 | 1.25 | 12 | 5/0.5 | 120 | 4 | 33 | 85 | 93.8 | 3.4 |
| 10 | 1.25 | 6 | 5/0.5 | 100 | 8 | 51 | 85 | 96.7 | 1.2 |
| 11 | 2.5 | 3 | 5/0.5 | 120 | 4 | 32 | 75 | 96.5 | 1.9 |
| 12 | 2.5 | 6 | 5/0.5 | 120 | 4 | 32 | 92 | 94.4 | 3.0 |
| 13 | 2.5 | 9 | 5/0.5 | 120 | 4 | 33 | 89 | 96.1 | 2.4 |
| 14* | 2.5 | 6 | 5/0.5 | 120 | 4 | 33 | 29 | 99.1 | 0.5 |

Conversion was determined as described in Method 5, Example 11. Selectivity was determined as described in Method 2, Example 11. Conditions: Substrate (300 mg), AcOH, DMA (3 mL) and Pd(OAc)₂; the reaction mixtures were heated in the absence of the substrate for 10 to 20 min at a temperature of 140° C. before the continuous flow reaction.

*The reaction mixture was directly injected into the flow reactor without prior generation of palladium(0) particles.

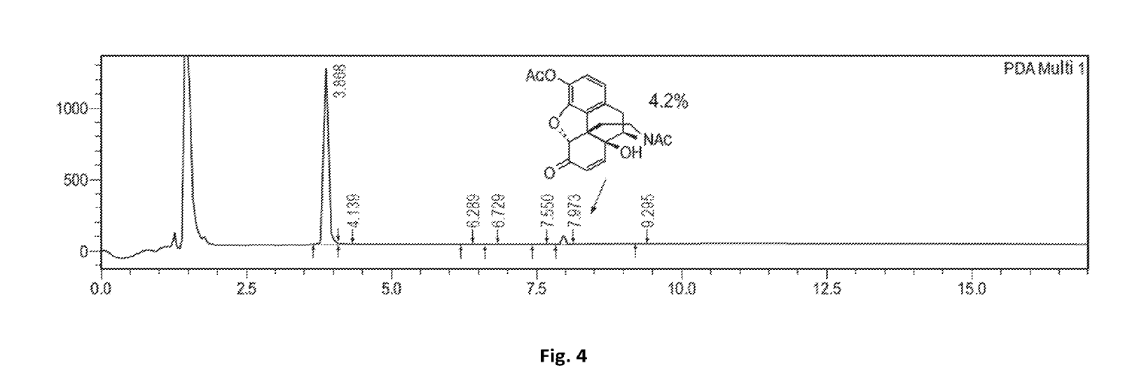

The reaction was finally repeated under the best conditions (2.5 mol % Pd(OAc)₂, 6 equivalents of AcOH) on a 6 gram scale. The product was formed with good selectivity and high conversion (98%) in this reaction. The main side-product in this reaction was the 3,17-diacetyl-14-hydroxynormorphinone (4.2%). FIG. 4 shows a HPLC-UV/Vis chromatogram of the product containing reaction mixture.

### (5aR,6R,8aS,8a1S,11aR)-11-oxo-5,5a,11,11a-tetrahydro-7H-6,8a1-ethanofuro[2′,3′,4′,5′:4,5]phenanthro[9,8a-d]oxazol-2-yl acetate (oxazolidine-containing Compound 6)

¹H NMR (300 MHz, DMSO) δ 6.91 (d, J=8.2 Hz, 1H), 6.85-679 (m, 2H), 6.21 (d, J=10.1 Hz, 1H), 4.88 (s, 1H), 4.76 (d, J=5.7 Hz, 1H), 4.62 (d, J=5.0 Hz, 1H), 3.53 (d, J=7.4 Hz, 1H), 3.35 (d, J=19.3 Hz, 1H), 3.19 (dd, J=19.6, 7.6 Hz, 1H), 2.86-2.64 (m, 2H), 2.40-2.26 (m, 1H), 2.25 (s, 3H), 1.37 (dd, J=12.8, 3.9 Hz, 1H).

### Example 8

### Hydrolysis of the Oxazolidine-Containing Compound 4

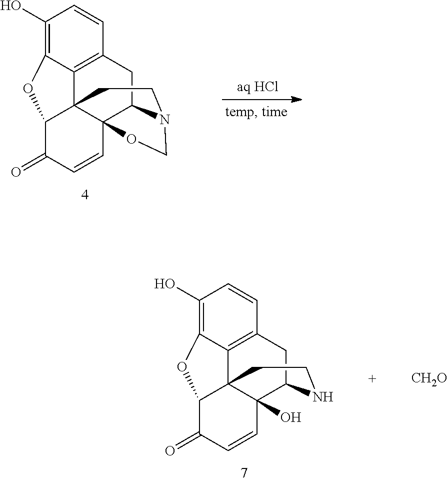

First hydrolysis reactions were performed with the isolated oxazolidine-containing compound 4 in either aqueous HCl or in DMA/aqueous HCl as solvent. After the reaction, the reaction mixture was brought to ˜pH 8 with 25% aqueous NH₃ to precipitate the product and the precipitate was isolated by filtration. The obtained product was analyzed by ¹H NMR spectroscopy and, further, by HPLC after derivatization with an excess of benzyl bromide (see Method 3 in Example 11). Below the boiling point of water, the hydrolysis reaction was slow with conversions of around 50% after a reaction time of 80 min at 80° C. in 1 n HCl. Higher concentrations of HCl increased the reaction rate, but the reaction selectivity decreased and several unidentified side-products were formed. The reaction selectivity was significantly increased when DMA/H₂O 1:1 was used as the solvent (Entry 4 and 5 in Table 15).

Above the boiling point of water, the reaction was fast with conversion of >90% after reaction times of only 10 min (Entry 10 and 11 in Table 15). The selectivity increased at temperatures above 100° C. It is theorized that some of the detected side-products were formed by a reaction of the electron rich A ring of the oxazolidine-containing compound 4 or 14-hydroxynormorphinone 7 with formaldehyde. Thus, efficient removal of formaldehyde is theorized to be important to drive the equilibrium to the hydrolyzed product and to prevent side reactions with CH₂O. The use of toluene as co-solvent to strip off CH₂O from the reaction mixture had no effect (Entry 13 in Table 15).

TABLE 15
Hydrolysis of oxazolidine 4.

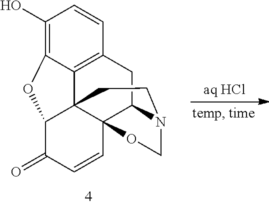 [SMILES: O=C1C=C[C@]23OCN4CC[C@@]25c2c(ccc(O)c2O[C@@H]15)C[C@@H]43]

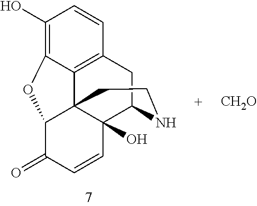 [SMILES: O=C1C=C[C@@]2(O)[C@H]3Cc4ccc(O)c5c4[C@@]2(CCN3)[C@H]1O5 ; "CH2O"]

| | HC1 | temp | time | conv/sel | conv. | yield |
|---|---|---|---|---|---|---|
| | [M] | [° C.] | [min] | (HPLC) [%] | (¹H NMR) [%] | [mg] |
| 1 | 2 | 80 | 80 | 60/68 | 55 | 47 |
| 2 | 1 | 80 | 80 | 53/79 | 50 | 56 |
| 3 | 0.5 | 80 | 80 | 26/77 | 22 | 43 |
| 4 | 2 | 80 | 80 | 73/93 | 67 | 46 |
| 5 | 1 | 80 | 80 | 26/96 | 24 | 51 |
| 6 | 2 | 100 | 40 | 92/78 | 87 | 48 |
| 7 | 1 | 100 | 40 | 77/81 | 77 | 56 |
| 8 | 2 | 110 | 40 | 97/76 | complete | 44 |
| 9 | 1 | 110 | 40 | 94/84 | complete | 54 |
| 10 | 2 | 120 | 20 | 100/70 | complete | 42 |
| 11 | 1 | 120 | 20 | 98/84 | complete | 53 |
| 12 | 1 | 120 | 10 | 92/89 | — | 58 |
| 13 | 1 | 120 | 10 | 90/88 | — | 60 |

Conversion was determined as described in Method 3, Example 11 (HPLC) or ¹H-NMR analysis. Conditions: 60 mg oxazolidine 4 in 600 μL aqueous HCl. Product was precipitated with aqueous ammonia after the reaction and isolated by filtration. conv=conversion of oxazolidine; sel=selectivity for hydroxynormorphinone 7. yield=yield of the solid after precipitation. Experiment 4 and 5 were done in DMA/H₂O 1:1 as solvent; experiment 13 was done with toluene as co-solvent.

To facilitate hydrolysis the reaction was performed under reduced pressures. The reaction was considerably faster under reduced pressures. While the conversion was 53% after 80 min at 80° C. under atmospheric pressure, 77% conversion was obtained after only 4 min at 80° C. at 140 mbar (Entry 4 in Table 16). The reaction selectivity was around 88%.

TABLE 16
Hydrolysis of oxazolidine 4 under reduced pressure in 1N HCl.

| | | | | conv/sel | yield |
|---|---|---|---|---|---|
| | temp [° C.] | time [min] | pressure [mbar] | (HPLC) [%]^{a} | [mg]^{b} |
| 1 | 60 | 10 | 100 | 31/77 | 49 |
| 2 | 80 | 4 | 240 | 62/89 | 53 |
| 3 | 80 | 10 | 240 | 69/87 | 53 |
| 4 | 80 | 4 | 140 | 77/88 | 49 |

Conversion (conv) and selectivity (sel) was determined as described in Method 3, Example 11. Conditions: 60 mg oxazolidine 4 in 600 μL 1 N aqueous HCl. conv=conversion of oxazolidine; sel=selectivity for hydroxynormorphinone 7. yield=yield of the solid after precipitation.

### Example 9

### Combined Palladium Catalyzed Oxidation and Hydrolysis

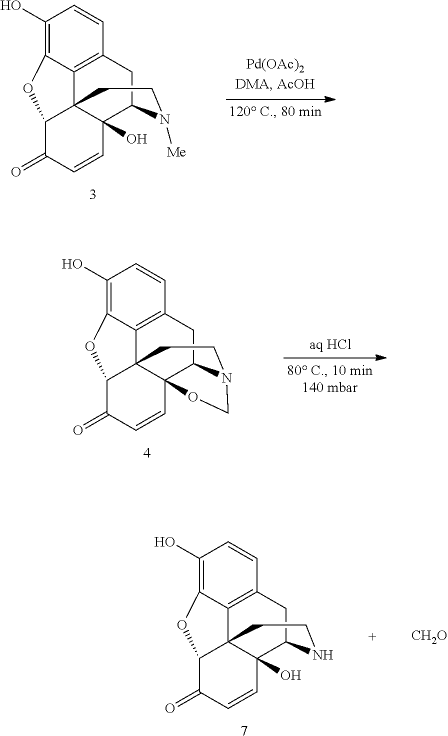

The oxazolidine-containing compound 4 was prepared by aerobic oxidation as described in Example 4 and 5. The product precipitated as a brown solid during the reaction. The conversion of the starting material to the product was >99% after 80 min at 120° C. (500 mg scale). 600 μL of this mixture were taken and diluted with the desired amounts of aqueous HCl. Upon addition of aqueous HCl the mixture became homogenous. The subsequent hydrolysis of these mixtures was done in a rotavapor for 10 min at 80° C. at a pressure of 140 mbar. Afterwards, the products were precipitated by the addition of 25% NH₃ and analyzed by HPLC-UV/Vis after derivatization with benzyl bromide. The selectivity for the product according to this analysis was ˜90% in all cases (Table 17). Similar results were obtained with H₂SO₄ instead of HCl.

TABLE 17
Oxidation and hydrolysis of 14-hydroxymorphinone 3

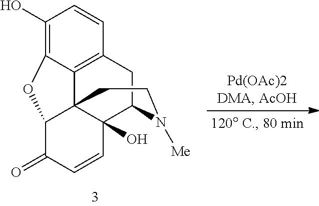 [SMILES: CN1CC[C@]23c4c5ccc(O)c4O[C@H]2C(=O)C=C[C@@]3(O)[C@H]1C5]

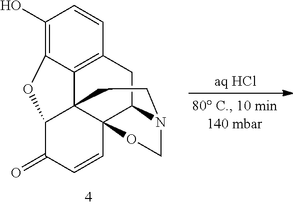 [SMILES: O=C1C=C[C@]23OCN4CC[C@@]25c2c(ccc(O)c2O[C@@H]15)C[C@@H]43]

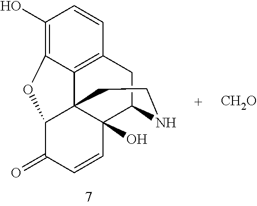 [SMILES: O=C1C=C[C@@]2(O)[C@H]3Cc4ccc(O)c5c4[C@@]2(CCN3)[C@H]1O5 ; "CH2O"]

| | HCl | 3 | 4 | 7 | yield |
|---|---|---|---|---|---|
| | volume (mL)/[M] | (HPLC) [%] | (HPLC) [%] | (HPLC) [%] | [mg]^{b} |
| 1 | 0.6 mL/1 M | <1 | 2 | 92 | 53 |
| 2 | 1.2 mL/1 M | <1 | 4 | 88 | 24 |
| 3 | 1.8 mL/1 M | <1 | 4 | 89 | 16 |
| 4 | 1.2 mL/0.5 M | 1 | 4 | 88 | 51 |
| 5 | 1.8 mL/0.3 M | <1 | 5 | 89 | 42 |

Reaction composition was determined as described in Method 3, Example 11. Conditions: Oxidation: 500 mg 14-hydroxymorphinone, 5 mol % Pd(OAc)₂, 3 equiv AcOH in 5 mL DMA as solvent; 80 min at 120° C. Hydrolysis: 600 μL of the mixture plus aqueous HCl were heated for 10 min at 80° C. in a rotavapor at 140 mbar. yield=yield of the solid after precipitation.

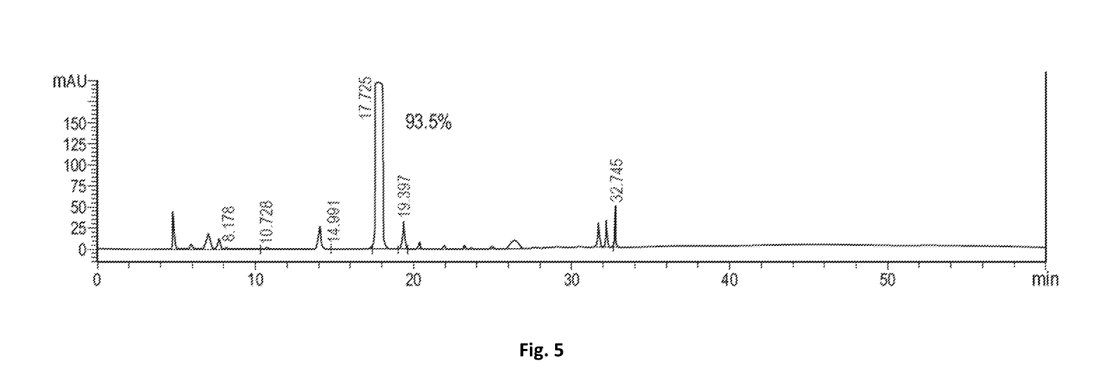

The reaction was repeated on a 500 mg scale with a reaction time for the oxidation of 120 min at a reaction temperature of 120° C. The reaction time for the subsequent hydrolysis was 20 min at 80° C. at a pressure of 140 mbar. The product was isolated by precipitation in 468 mg product yield (98% yield; 93.5% purity according to HPLC-UV/Vis at 205 nm). FIG. 5 shows a representative HPLC-UV chromatogram of the isolated product.

14-Hydroxynormorphinone (7):

¹H NMR (300 MHz, DMSO) δ 6.85 (d, J=10.1 Hz, 1H), 6.56-6.48 (m, 2H), 5.99 (d, J=10.0 Hz, 1H), 4.57 (s, 1H), 3.07 (t, J=3.6 Hz, 1H), 2.85 (d, J=3.5 Hz, 2H), 2.66 (dd, J=13.6, 4.4 Hz, 1H), 2.42 (dd, J=13.4, 3.5 Hz, 1H), 2.26 (td, J=12.2, 5.0 Hz, 1H), 1.27 (dd, J=12.1, 2.6 Hz, 1H).

### Example 10

### Combined Palladium Catalyzed Oxidation, Hydrolysis and Hydrogenation

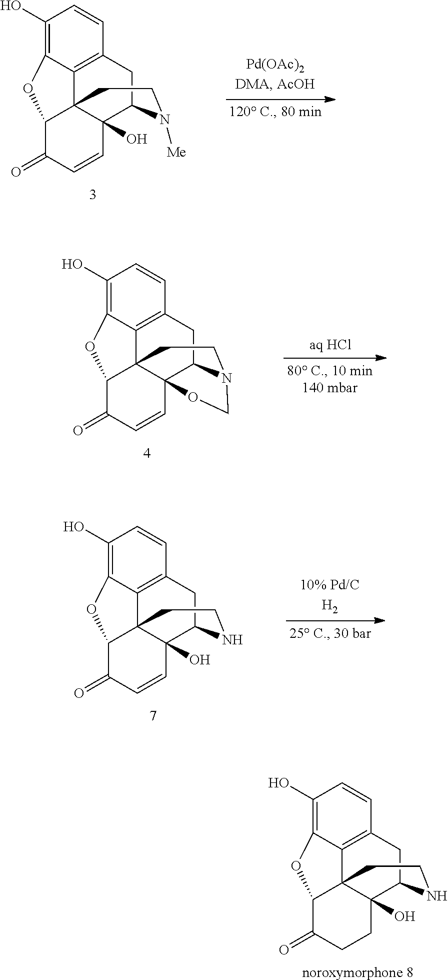

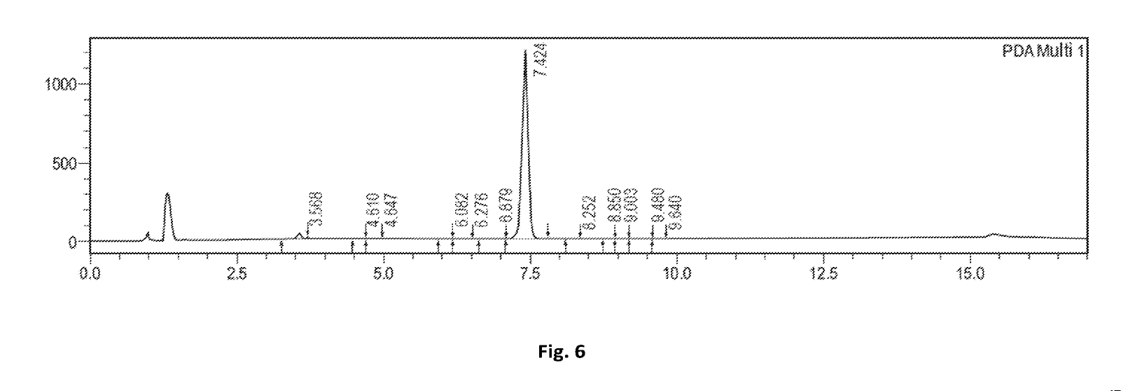

A continuous flow reaction was performed with 14-hydroxynormorphinone 3 on a 600 mg scale as described in Example 5. The solution of colloidal palladium(0) was prepared by heating Pd(OAc)₂ (1.25 mol %), acetic acid (3 equivalents) in 9 mL DMA for 20 min at 140° C. The substrate (600 mg) was dissolved in this mixture, and the mixture was pumped through the flow reactor at a flow rate of 1 mL/min. The solution was combined with the O₂ feed at a flow rate of 10 mL/min. The quench solution of 1N HCl was pumped with a flow rate of 1 mL/min. The reaction mixture left the system after a residence time of 19 min through the back pressure regulator (7 bar). The collected mixture was heated for 20 min at 80° C. at a pressure of 140 mbar to hydrolyze the oxazolidine-containing compound 4 to the 14-hydroxynormorphinone 7. The obtained mixture was finally pumped through the Thales H-Cube pro to hydrogenate the 14-hydroxynormorphinone 7 to the noroxymorphone 8. A complete conversion was obtained with a flow rate of 0.4 mL/min with a 10% Pd/C cartridge at a reaction temperature of 25° C. and a H₂ pressure of 30 bar. The collected sample was diluted with the same amount of distilled water and the noroxymorphone 8 was precipitated with aqueous NH₃. The noroxymorphone 8 was isolated in 403 mg (70% yield, 90.2% purity according to analysis as described in Method 5, Example 1). FIG. 6 shows a representative HPLC-UV chromatogram of the isolated product after derivatization with acetic anhydride.

Noroxymorphone (8):

¹H NMR (300 MHz, DMSO) δ 6.56 (d, J=8.1 Hz, 1H), 6.51 (d, J=8.1 Hz, 1H), 4.68 (s, 1H), 2.99-2.78 (m, 4H), 2.65-2.56 (m, 1H), 2.46-2.23 (m, 3H), 2.07 (d, J=14.2 Hz, 1H), 1.72 (d, J=11.6 Hz, 1H), 1.42 (td, J=14.0, 3.2 Hz, 1H), 1.15 (d, J=10.1 Hz, 1H).

### Example 11

### Preparation of Oxymorphone

A suspension of 14-hydroxymorphinone (750 mg, 2.5 mmol) and 10% Pd/C (25 mg, 1 mol %) in DMA was stirred under H₂ atmosphere for 1 h. Then, the reaction mixture was filtered using a syringe filter (PTFE, 0.45 μm) and the filtrate evaporated under reduced pressure.

### Example 12

### Sequential Hydrogenation/Oxidative Cyclization of 14-Hydroxymorphinone Using Colloidal Pd(0)

Continuous-flow Setup. The flow reactor consisted of an HPLC pump for introducing the liquid feed (Uniqsis Pump Module). Oxygen gas from a gas cylinder was fed into the system via a mass flow controller (Bronkhorst EL-Flow). The liquid and gaseous streams were combined in a T-mixer (stainless steel). The T-mixer was connected to the residence tube reactor via a fluoropolymer tubing (PFA, 1/16″ o.d., 0.8 mm i.d.). The PFA tubing allowed visual observation of the flow profile. The residence coil reactor (FEP, ⅛″ o.d., 1.6 mm i.d. 28 mL) was heated in a GC oven to the desired temperature. The reaction mixture finally exited the system through a short cooling loop (stainless steel, 1/16″ o.d., 1 mm i.d.) and a back-pressure regulator (adjustable back-pressure regulator, Vapourtec). Pressure sensors were integrated into the T-mixer and directly after the pump.

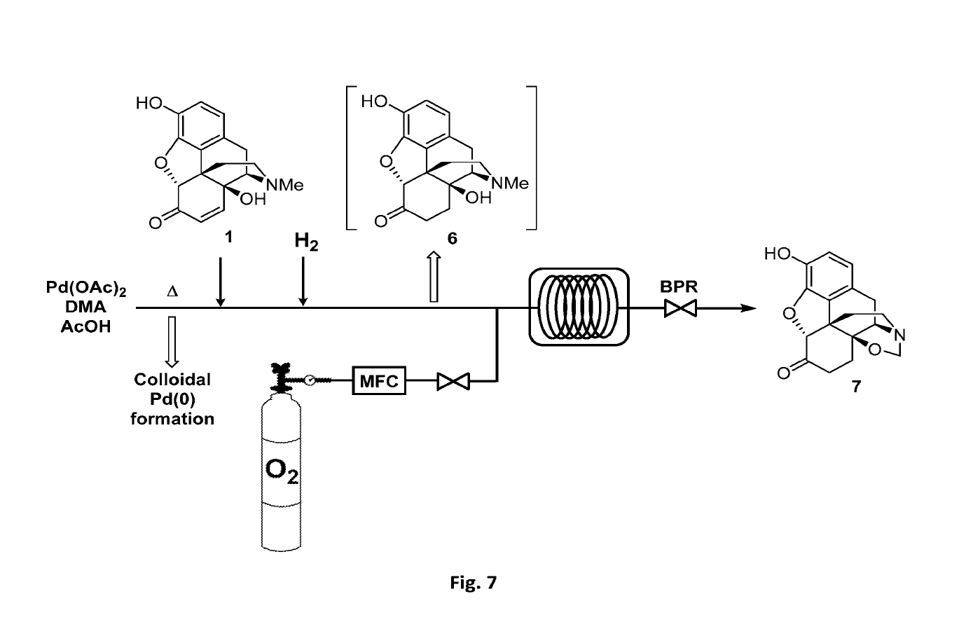

FIG. 7 shows the synthetic scheme for the preparation of 1,3-oxazolidine 7 by hydrogenation/oxidative cyclization catalyzed by colloidal Pd(0). Pd(OAc)₂ (3 mol %) and AcOH (3 equiv) were dissolved in DMA (5 mL) and placed in a 25 mL two-necked round-bottom flask. The mixture was heated at 120° C. for 15 min. Formation of Pd(0) could be visually observed. Upon cooling, 14-hydroxymorphinone (1) (150 mg, 0.5 mmol) was added and the mixture stirred under H₂ atmosphere for 1 h to form oxymorphone (6). Then, the crude reaction mixture was introduced into the flow reactor via an injection loop. The flow reactor had been stabilized by pumping DMA at a flow rate of 0.75 mL/min and O₂ with a flow rate of 1.7 mL/min (gas flow at normal conditions, i.e., TN=0° C. and pN=1 atm). The temperature of the GC-oven had been set to 120° C. and the pressure adjusted to 5 bar. After a residence time of approximately 20 min the processed product solution left the system. The reaction mixture collected from the reactor output was evaporated under reduced pressure until ca. 20% of the initial volume. 10 mL of cold water were added, and the solid obtained which contained 1,3-oxazolidine (7) was filtered and dried under vacuum at 50° C. (115 mg, 77%).

### Example 13

### Sequential Hydrogenation/Oxidative Cyclization of 14-Hydroxymorphinone Using Heterogeneous Catalysis

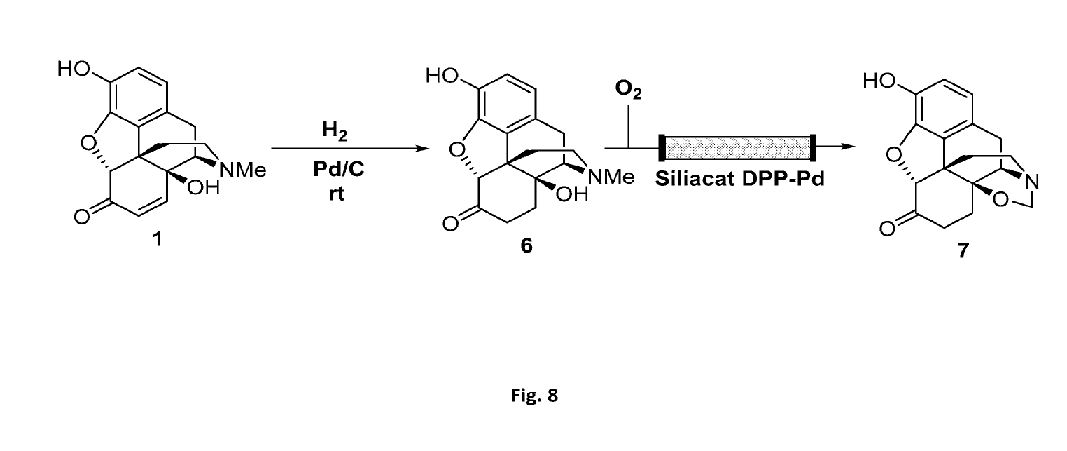

A suspension 14-hydroxymorphinone (150 mg, 0.5 mmol) and 10% Pd/C (10 mg, 1 mol %) in 5 mL DMA/ethylene glycol 9:1 was stirred at room temperature under H₂ atmosphere for one hour to form oxymorphone (6). The mixture was filtered (0.45 m pore size filter) and the clear solution was introduced into the flow reactor via an injection loop. The flow reactor had been stabilized by pumping DMA/ethylene glycol 8:2 at a flow rate of 0.4 mL/min and O₂ with a flow rate of 2.05 mL/min (gas flow at normal conditions, i.e. TN=0° C. and pN=1 atm). The temperature of the packed-bed reactor had been set to 120° C. and the pressure adjusted to 5 bar. After a residence time of approximately 20 min the processed product solution left the system. The reaction mixture collected from the reactor output was evaporated under reduced pressure until ca. 20% of the initial volume. 10 mL of cold water were added, and the solid obtained which contained 1,3-oxazolidine (7) was filtered and dried under vacuum at 50° C. (102 mg, 68%). FIG. 8 shows the hydrogenation/oxidative cyclization sequence for the preparation of 1,3-oxazolidine 7 using heterogeneous catalysts.

(5aR,6R,8aS,8a1S,11aR)-2-hydroxy-5,5a,9,10-tetrahydro-7H-6,8a1-ethanofuro[2′,3′,4′,5′:4,5]phenanthro[9,8a-d]oxazol-11(11aH)-one: mp Xxx ° C.; 1H NMR (300 MHz, DMSO-d6) δ 9.24 (s, 1H), 6.60 (q, J=8.1 Hz, 2H), 4.87 (s, 1H), 4.61 (dd, J=32.1, 5.5 Hz, 2H), 3.25-3.06 (m, 3H), 2.91-2.55 (m, 3H), 2.38-2.24 (m, 1H), 2.20 (dt, =7.3, 4.0 Hz, 1H), 1.89 (dt, J=7.3, 4.0 Hz, 1H); 1.47 (td, J=14.8, 2.9 Hz, 1H), 1.25 (dd, J=12.4, 4.0 Hz, 1H). 13C NMR (75 MHz, DMSO) δ 208.2, 143.6, 139.7, 129.7, 122.1, 120.0, 118.1, 90.5, 86.1, 77.1, 63.6, 52.5, 44.1, 37.3, 34.4, 30.5, 26.6.

### Example 14

### Analysis

Method 1:

GC-MS spectra were recorded using a Thermo Focus GC coupled with a Thermo DSQ II (EI, 70 eV). A HP5-MS column (30 m×0.250 mm×0.025 μm) was used with Helium as carrier gas (1 mL min⁻¹ constant flow). The injector temperature was set to 280° C. After 1 min at 50° C. the temperature was increased in 25° C. min⁻¹ steps up to 300° C. and kept at 300° C. for 4 minutes. Peak area integration was used to assess conversion of the substrate to the product.

Method 2:

Analytical HPLC-UV/Vis (Shimadzu LC20) analysis was carried out on a C18 reversed-phase (RP) analytical column (150×4.6 mm, particle size 5 μm) at 37° C. using a mobile phase A (water/acetonitrile 90:10 (v/v)+0.1% TFA) and B (MeCN+0.1% TFA) at a flow rate of 1.5 mL min⁻¹. The detection wavelength was set at 215 nm. The following gradient was applied: linear increase from solution 3% B to 25% B in 9 min, followed by linear increase from solution 25% B to 80% B in 7 min, hold at 80% B for 1 min. Peak area integration was used to assess conversion and reaction selectivity.

Method 3:

25 μL of benzyl bromide were added to 50 μL of the reaction mixture, and the sample was heated for 60 min at 70° C. The sample was diluted with 1 mL MeCN and analyzed by HPLC-UV/Vis. Analytical HPLC-UV/Vis analysis was carried out as described in Method 2. The detection wavelength was set at 280 nm.

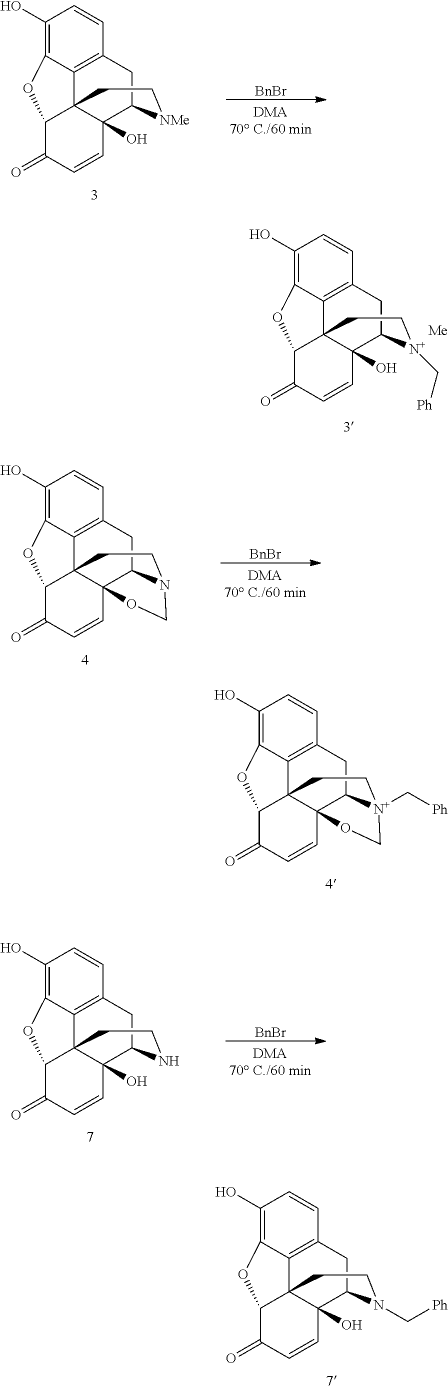

Method 4:

LCMS analysis (Shimadzu LC20) was carried out with a C18 reversed-phase (RP) analytical column (150×4.6 mm, particle size 5 μm) at room temperature using a mobile phase A (water/acetonitrile 90:10 (v/v)+0.1% formic acid) and B (MeCN+0.1% formic acid) at a flow rate of 0.6 mL min⁻¹. The following gradient was applied: linear increase from solution 5% B to 100% B in 20 min. The LC was connected to a mass spectrometer. The low-resolution mass spectra were obtained on a Shimadzu LCMS-QP2020 instrument using electrospray ionization (ESI) in positive or negative mode. 50 μL of the reaction mixture were diluted with 1 mL MeCN and the sample was analyzed by LCMS. The m/z values of 286 and 300 were extracted from the total ion current chromatograms. The peak areas of the extracted-ion chromatograms were then used to calculate conversion of the substrate to the product.

Method 5:

50 μL of the reaction mixture were pipetted to 500 μL sat NaHCO₃. 150 μL of Ac₂O were then added, and the mixture was stirred for 15 min at 60° C. The mixture was diluted with 500 μL MeCN and analyzed by HPLC-UV/Vis. Analytical HPLC-UV/Vis analysis was carried out as described in Method 2. The detection wavelength was set at 215 nm.

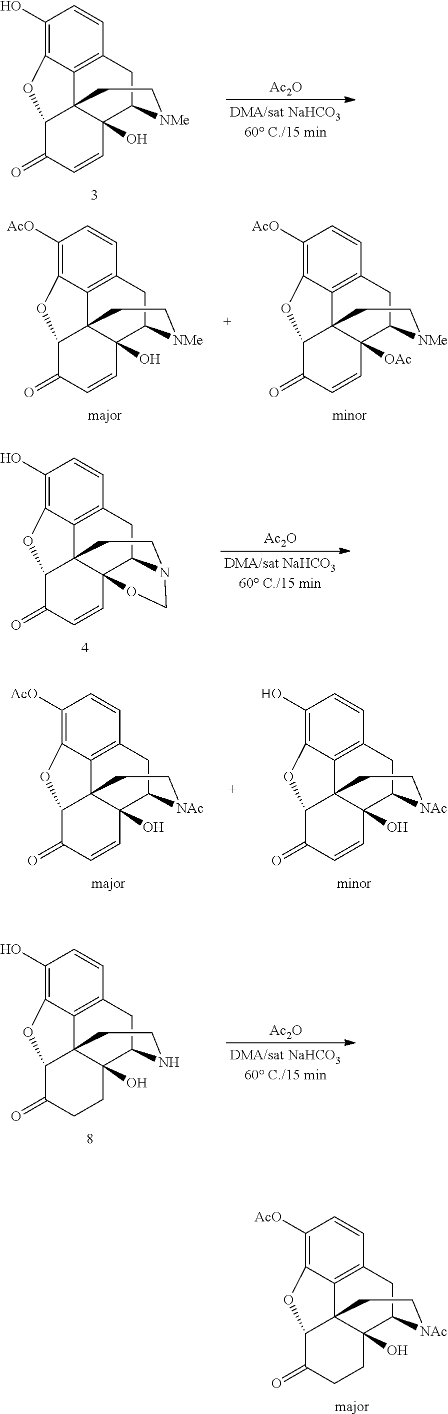

Method 5:

50 μL of the reaction mixture were pipetted to 500 μL sat NaHCO₃. 150 μL of Ac₂O were then added, and the mixture was stirred for 15 min at 60° C. The mixture was diluted with 500 μL MeCN and analyzed by HPLC-UV/Vis. Analytical HPLC-UV/Vis analysis was carried out as described in Method 2. The detection wavelength was set at 215 nm.

### Example 15

### Continuous Flow Sequential Hydrogenation/Aerobic Oxidation of 14-Hydroxymorphinone Using Colloidal and Immobilized Palladium Catalysts

N-Demethylation of morphine and other opiate analogs is a key step for the preparation of many analgesics, opioid antagonists, and other related drugs, as modification of the N-methyl group has a significant impact on their pharmacological properties. Naloxone and naltrexone, which are among the most important opioid antagonists available, can be prepared from 14-hydroxymorphinone 1 after hydrogenation of the 7,8-alkene and a demethylation/alkylation sequence (Scheme 6). 14-Hydroxymorphinone 1, in turn, can be directly synthesized from naturally occurring oripavine extracted from poppy plants.

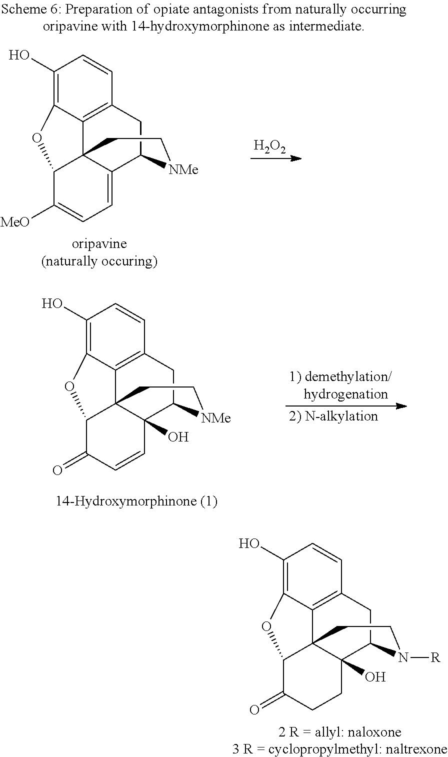

Traditional N-demethylation procedures include the use of strong electrophiles such as chloroformates or cyanogen bromide, giving carbamate or cyanamide intermediates that are subsequently hydrolyzed. Alternative formation of the corresponding N-oxides using peracids followed by dehydration and hydrolysis of the ensuing iminium compounds have also been proposed. Unfortunately, these methods involve the use of stoichiometric amounts of toxic and corrosive reagents and generate large amounts of waste.

In 2008 Hudlicky and coworkers developed an elegant method for the palladium catalyzed demethylation of hydrocodone using molecular oxygen as oxidant. Using 10 mol % Pd(OAc)₂ as catalyst and dioxane/acetic anhydride as solvent, N-acetyl norhydrocodone was obtained after 15 h at 80° C. at atmospheric pressure. The same methodology did not perform well however for other opiates including morphine, codeine, and oxycodone. More recently, Kappe and coworkers intensified the process and extended the scope of this demethylation strategy under continuous flow conditions. Using Pd-black colloidal particles (generated in situ from Pd(OAc)₂) in DMA, 14-hydroxymorphinone 1 could be oxidized to the unexpected cyclic 1,3-oxazolidine structure 4 (Scheme 7), which after hydrolysis and hydrogenation over Pd/C gave noroxymorphone 5. Notably, under flow conditions, 96% conversion and >90% selectivity was obtained for the oxidative cyclization step after only 20 min residence time at 120° C. and 1.25 mol % catalyst loading. The high efficiency for the oxidation process was attributed to the excellent mass transfer and well defined interfacial gas-liquid contact areas achieved with a segmented flow regime.

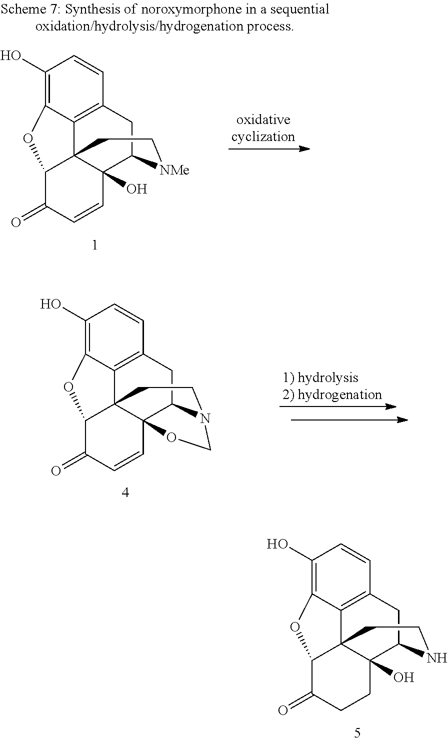

Despite the purity and overall yield for noroxymorphone 5 obtained by Kappe and coworkers using the continuous flow setup, there were some drawbacks mainly related to the relatively high reactivity of the α,β-unsaturated carbonyl group of the substrate 1 and its 1,3-oxazolidine derivative 4, especially during the hydrolysis step, which apparently produced most of the observed side products. Moreover, although the catalyst loading could be reduced in comparison to the batch experiments, a second source of the metal was needed to perform the last hydrogenation step. In this context, an alternative procedure has been devised as described herein, in which the 7,8-dehydro group is initially reduced using in situ generated colloidal Pd(0). Then, the same catalyst is reused for the oxidative cyclization step in one pot using O₂ as oxidant. Furthermore, a novel methodology for the hydrogenation/oxidative cyclization sequence is introduced herein, using two separate heterogeneous Pd catalysts: Pd/C for the hydrogenation step, and for the oxidation heterogeneous SiliaCat DPP-Pd in a packed bed reactor.

Sequential hydrogenation/oxidative cyclization using colloidal Pd(0). Colloidal Pd(0) particles are readily generated in solution when a Pd source (e.g. Pd(OAc)₂) is heated at temperatures above 100° C. In DMA as solvent using a small amount of AcOH as additive for stabilizing the colloidal Pd(0), the metal showed a very high efficiency for the oxidative cyclization of 14-hydroxymorphinone 1 to the 1,3-oxazolodine 4 (cf. Scheme 7). Colloidal Pd(0) particles are known to promote catalytic hydrogenations as well. A one-pot procedure for the sequential hydrogenation/oxidation of 14-hydroxymorphinone 1 was devised (FIG. 7), under flow conditions to obtain the 1,3-oxazolidine 7, the direct precursor of noroxymorphone. Additionally, initial removal of the 7,8-alkene moiety was expected to improve the purity of the final product.

Continuous flow experiments were performed using a simple 2-feed approach. Thus, the liquid and gas feed were mixed using a stainless steel T-mixer before entering the reactor, which consisted of PFA tubing (1.6 mm inner diameter, 28 mL volume) heated at the desired temperature using a GC-oven. The liquid feed was pumped using an HPLC-pump, while oxygen was accurately introduced into the reactor using a mass flow controller (Bronkhorst EL-Flow). The system was pressurized using an adjustable back-pressure regulator (Vapourtec). In a typical experiment Pd(OAc)₂ (1-3 mol %, see Table 18) and AcOH were dissolved in DMA (5 mL). The solution was heated under stirring at 120° C. for ca. 15 min, resulting in a dark mixture containing the colloidal Pd(0). Upon cooling, 14-hydroxymorphinone (1 mmol) was added and the mixture stirred under H₂ atmosphere for 1 h. Then, the reaction mixture was directly processed in the oxidation flow setup, introducing the homogeneous crude mixture to the reactor via a sample loop.

As expected, hydrogenation of 14-hydroxymorphinone 1 to oxymorphone 6 using the in situ generated colloidal Pd(0) performed very well, and full conversion was observed after 1 h at atmospheric pressure for catalyst loadings ranging from 1 mol % to 3 mol %. HPLC analysis of the reaction mixture confirmed a highly selective reduction of the double bond, with no traces of side products being observed. Gratifyingly, processing of the crude hydrogenation reaction mixture through the aerobic oxidation flow setup resulted in formation of the desired 1,3-oxazolidine derivative 7 with good conversions and selectivity, demonstrating that the colloidal Pd(0) can be utilized both for the hydrogenation and oxidation steps in an “one-pot” procedure. Several reaction parameters were screened to obtain optimal reaction conditions for the oxidation step (Table 18). The amount of AcOH used as additive had a significant influence on the conversion, the optimal amount being 3 equiv. A higher excess (entry 9) or small amounts (entry 11) clearly reduced the conversion. Notably, conversions above 90% were only achieved with catalyst loadings of 3 mol % after reaction times of approximately 20 to 30 min.

TABLE 18
Optimization of the reaction parameters for the continuous flow
oxidative cyclization of oxymorphone 6 to 1,3-oxazolidine 7

| | O₂ | AcOH | Pd(OAc)₂ | temp | p | res time | conv | sel |
|---|---|---|---|---|---|---|---|---|
| | [eq] | [eq] | [mol %] | [° C.] | [bar] | [min] | [%]^{[a]} | [%]^{[a]} |
| 1 | 3 | 3 | 3 | 120 | 9 | ~35 | 95 | 83 |
| 2 | 3 | 3 | 1 | 120 | 9 | 15-20 | 44 | 93 |
| 3 | 3 | 3 | 1 | 120 | 9 | ~30 | 67 | 85 |
| 4 | 1.5 | 3 | 1 | 120 | 5 | ~20 | 80 | 90 |
| 5 | 1.5 | 3 | 1 | 120 | 5 | ~25 | 79 | 84 |
| 6 | 1.5 | 3 | 1 | 120 | 3 | ~15 | 59 | 90 |
| 7 | 1.5 | 3 | 2 | 120 | 3 | ~20 | 89 | 87 |
| 8 | 1.5 | 3 | 2 | 120 | 5 | ~30 | 91 | 82 |
| 9 | 1.5 | 10 | 2 | 120 | 5 | ~25 | 60 | 70 |
| 10 | 1.5 | 1 | 2 | 120 | 5 | ~25 | 98 | 88 |
| 11 | 1.5 | 0.3 | 2 | 120 | 5 | ~25 | 83 | 88 |
| 12 | 1.5 | 0.3 | 3 | 120 | 5 | ~25 | 90 | 84 |
| 13 | 1.5 | 3 | 3 | 120 | 5 | ~25 | 99 | 85 |

^{[a]}Determined by HPLC area integration (205 nm) after derivatization with Ac₂O.

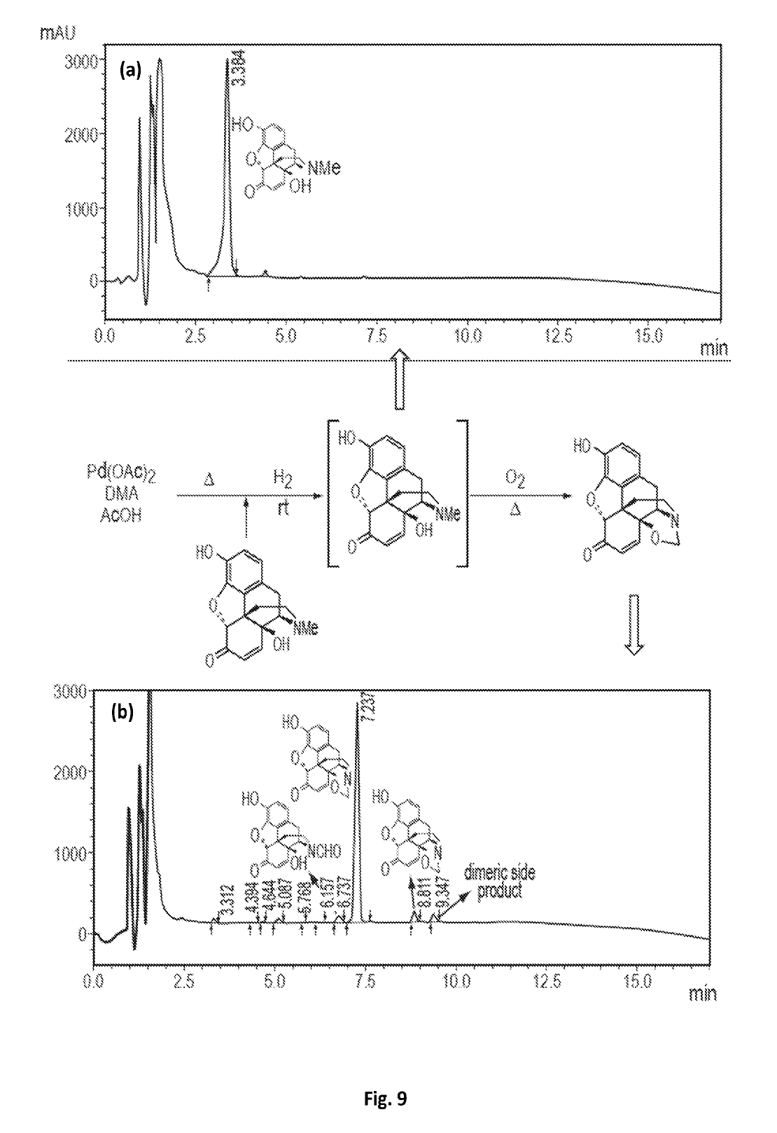

HPLC monitoring of the reaction sequence after the first and second step (FIG. 9) revealed that several side-products are formed during the oxidation, contributing to the moderate selectivity (80-90%) observed in most cases. Apart from partial reoxidation to the initial 7,8-dehydro group, which gives the corresponding cyclized 1,3-oxazolidine 4 (ca. 4% HPLC area), a compound with a mass corresponding to a dimeric structure was also observed (ca. 3% HPLC area; FIG. 9). Overoxidation to the carboxamide 8 was observed as well in some cases.

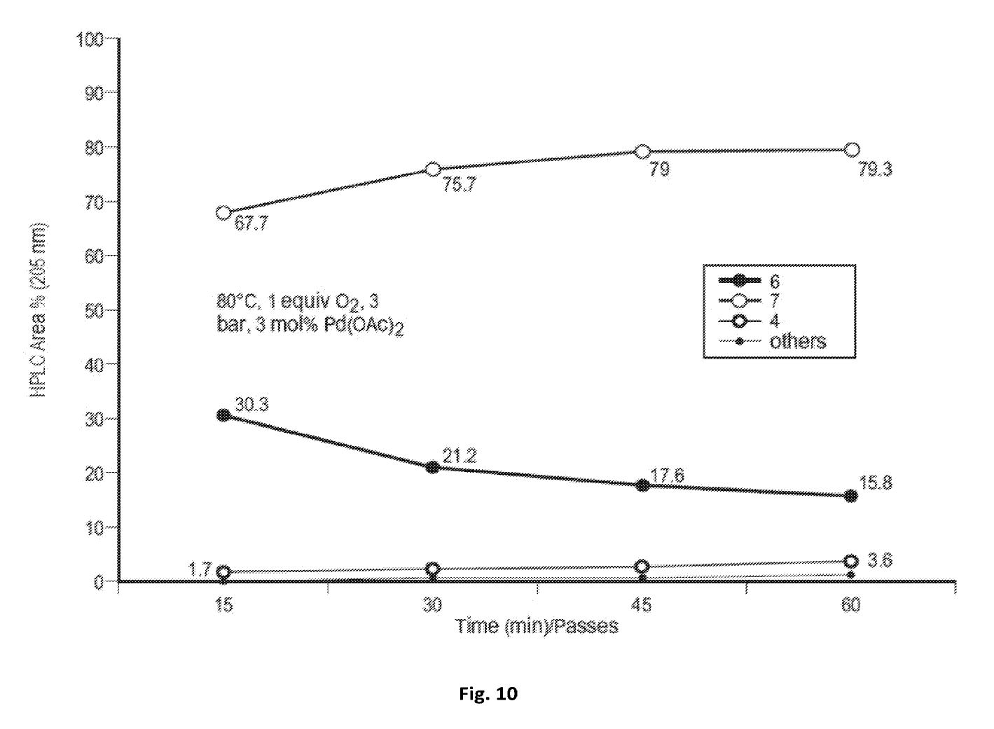

In an attempt to improve the selectivity for the formation of 7, reactions at lower temperatures were also performed (80° C. to 100° C.). Thus, to obtain good conversions the reaction mixture was processed several times through the flow reactor. Notably, this strategy was unsuccessful and the reaction stopped after a number of passes in all cases. At 80° C. and 5 bar pressure, for example (FIG. 10), the reaction conversion could not be improved after the mixture was processed 3 times. These results strongly support the hypothesis that the colloidal Pd(0) particles are deactivated in the presence of oxygen at high temperatures, most likely forming the initial Pd(OAc)₂. As shown previously, Pd(OAc)₂ is not an active catalyst for the reaction without the preactivation process to colloidal Pd(0).

A second possible source of decrease in catalytic activity could be the aggregation of the Pd(0) particles during the hydrogenation of 1. To test the catalytic activity of the freshly generated colloidal Pd(0), it was decided to directly use oxymorphone 6 as substrate (which had been previously prepared by hydrogenation of 14-hydroxymorphinone 1 over Pd/C) for the continuous flow oxidation process. Gratifyingly, after 20 min residence time at 100° C. using this methodology, 99% conversion of 6 and 94% selectivity was observed for the desired 1,3-oxazolodine 7. After simple evaporation of the crude reaction mixture to ca. 20% of the initial volume and addition of cold water, 7 crystallized in 77% yield. It therefore seems that several factors can alter the catalytic efficiency of colloidal Pd(0). On the one hand aggregation can reduce their efficiency, even in the presence of AcOH as stabilizing agent. Furthermore, some attempts to intensify the oxidation process increasing the amount of oxygen or pressure failed, most probably due to a more rapid decomposition of the Pd(0) particles under oxygen rich conditions.

Sequential hydrogenation/oxidative cyclization using heterogeneous Pd catalysts. An investigation of the hydrogenation and subsequent oxidation of 14-hydroxymorphinone 1 using colloidal Pd(0) (see above) revealed that the oxidation step performs best when the metal nanoparticles are freshly prepared. Thus, excellent results were obtained when oxymorphone 6, which had been previously prepared and isolated from hydrogenation of 14-hydroxymorphinone 1, was directly used as substrate. This procedure has the important drawback of requiring the isolation and handling oxymorphone 6, which increases solvent waste and is operationally more complex. A one-pot protocol for the sequential process instead would be a desired procedure. A new strategy for the sequential process in which two separate heterogeneous catalysts are used was devised. Thus, hydrogenation of the starting 14-hydroxymorphinone 1 was carried out over Pd/C (FIG. 8). Then, the resulting solution of oxymorphone 6 in DMA was directly subjected to continuous flow oxidation using a packed bed reactor containing SiliaCat DPP-Pd (Silicycle) as heterogeneous catalyst to obtain 7.

The continuous flow setup utilized for this process was analogous to the homogeneous procedure described above. In this case, after the T-mixer the coil reactor was substituted by a column reactor (Omnifit) which contained the sol-gel entrapped SiliaCat DPP-Pd. This catalyst consists of an organosilica matrix functionalized with diphenylphosphine ligands bound to Pd(II), and has been used for a wide range of Pd-catalyzed organic transformations. SiliaCat DPP-Pd was chosen due to the high leaching resistance shown by this catalyst for reactions involving heterogeneous Pd(0) and homogeneous Pd(II) species in the reaction mechanism (which is likely to occur for this oxidation reaction).

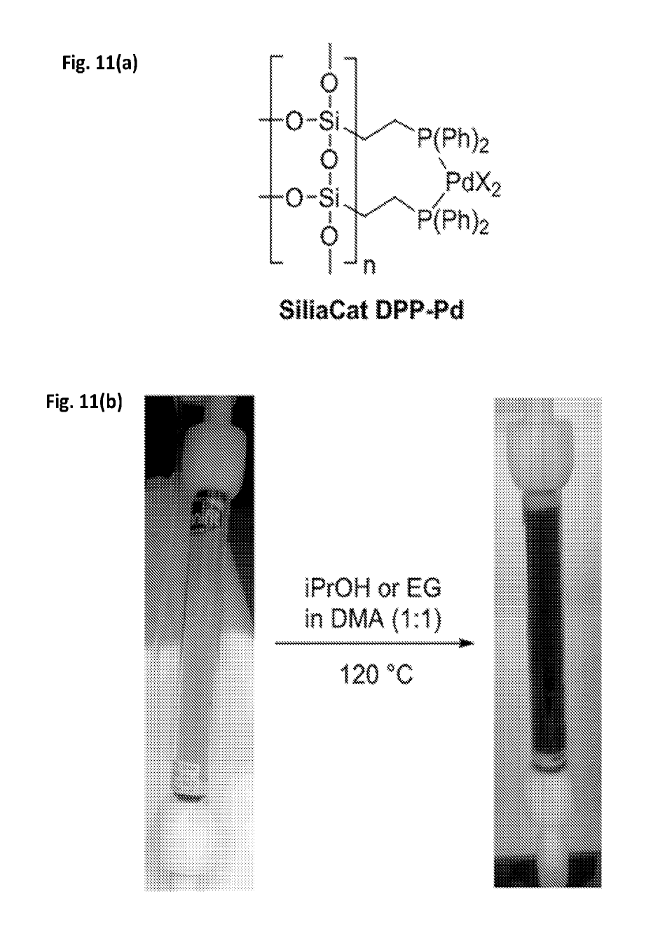

Preliminary experiments were performed using a 0.7 mL packed bed reactor containing 220 mg of the immobilized catalyst (0.055 mmol Pd). As commercial SiliaCat DPP-Pd is initially in a Pd(II) form, the catalyst was preactivated by pumping through the column at 120° C. a solution of isopropanol or ethylene glycol (EG) in DMA (50% vol). During this process, a rapid change in the solid catalyst color, from orange to black, could be visually observed demonstrating the transformation from Pd(II) to Pd(0) (FIGS. 11(a) and 11(b)). Ultimately ethylene glycol was selected as additive for the catalyst activation as more reproducible results were obtained compared to iPrOH. When a solution of oxymorphone 6 in DMA was processed using the packed bed reactor with the activated Pd catalyst (using a flow rate of 0.4 mL/min for the liquid phase, and 2.05 mL_{N}/min for O₂, corresponding to 1.5 mol equiv) 30% conversion to the desired 1,3-oxazolidine 7 was observed at 120° C. (HPLC analysis, 205 nm). However, HPLC monitoring of the reaction output during a 5 mL run revealed a rapid decrease in the reaction conversion. Moreover, the immobilized catalyst had partially recovered its initial orange coloration. After a new “catalyst activation” cycle using ethylene glycol in DMA at high temperature the oxidation could be repeated with identical result. This continuous flow oxidation/catalyst reactivation cycle could be repeated many times without apparent drop in catalytic efficiency. Thus, the observed decrease in conversion in each cycle was ascribed to transformation of the Pd immobilized in the support to inactive Pd(II) species instead of leaching.

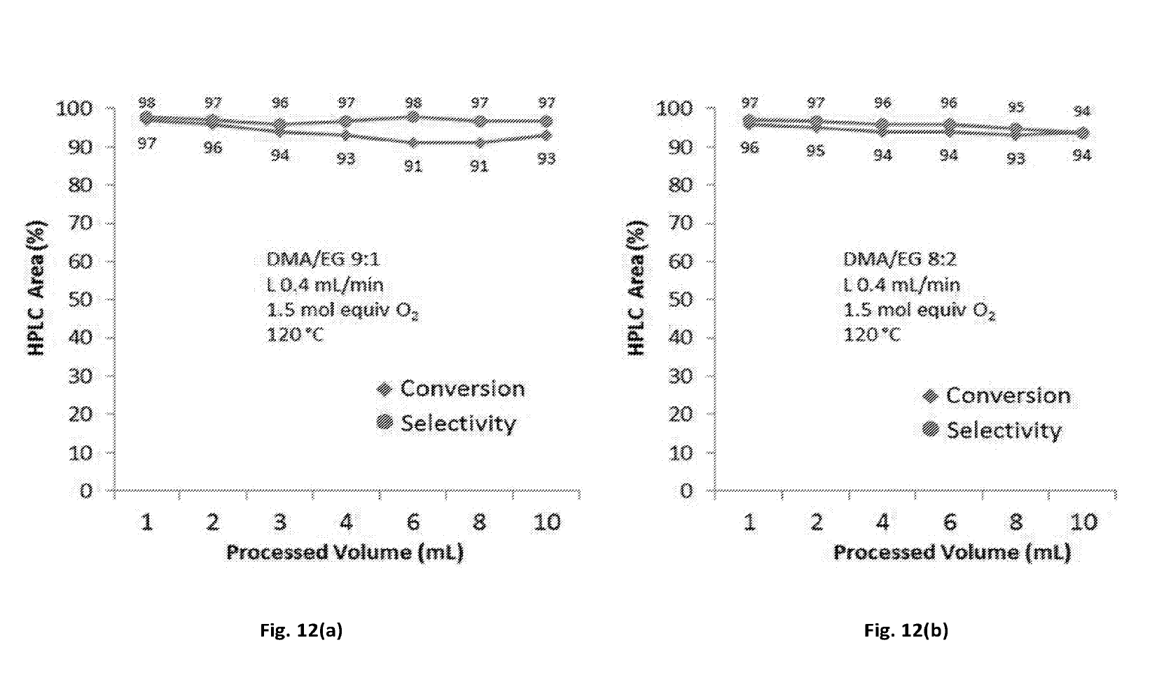

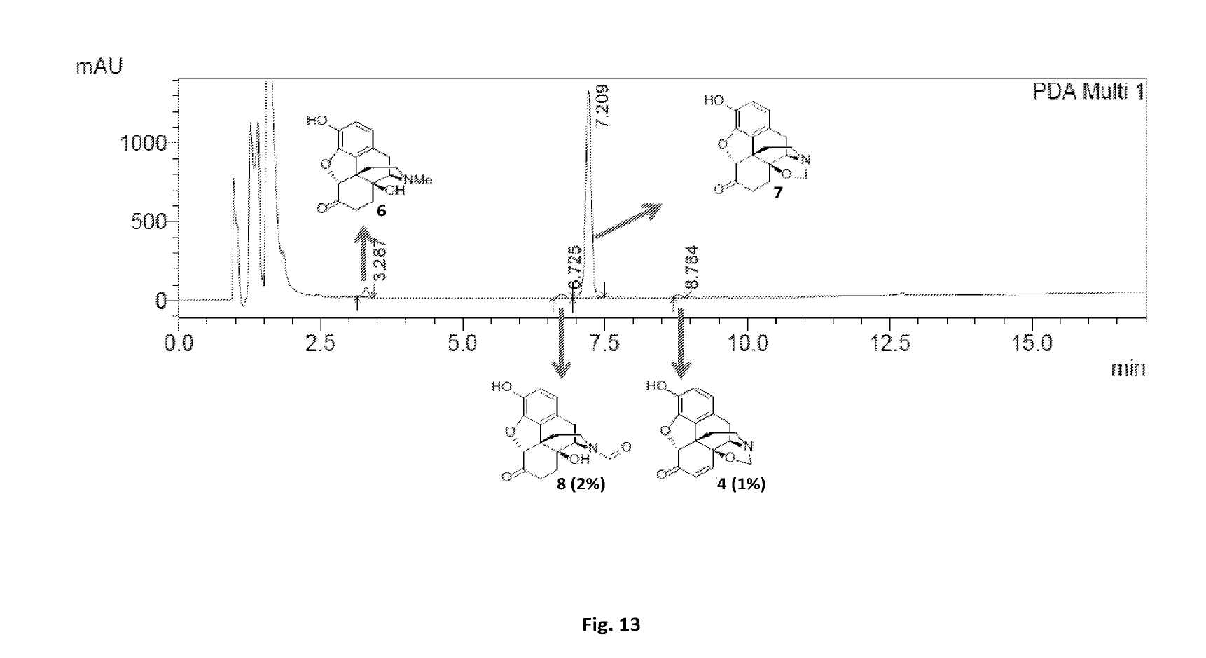

A larger packed bed reactor was used as well (2.4 mL volume, 760 mg SiliaCat DPP-Pd) to extend the residence time and improve the reaction conversion. Moreover, ethylene glycol was added as co-solvent to DMA to improve the immobilized catalyst stability, i.e., to reactivate the catalyst concurrently during the reaction, thus obtaining a constant conversion profile. With the larger packed-bed reactor high conversions and excellent selectivities were obtained (FIG. 12) and, importantly, improved catalyst stability. Using 10% EG in DMA as solvent system (FIG. 12a) an initial conversion of 97% (HPLC area) with 98% selectivity was obtained. Only minor amounts of two side products were observed: the over-oxidized carboxamide 8 and the 7,8-dehydro side product 4. No traces of dimer were detected in this case by HPLC monitoring (FIG. 13). 10 mL reaction volume were processed, during which the selectivity was constantly ca. 97-98% and the conversion oscillated, showing a drop to 91% after 6-7 mL had been flown through the column. Increasing the EG amount to 20% somewhat improved the stability (FIG. 12b), although a higher amount of 8 was observed by HPLC monitoring, thus decreasing the reaction selectivity.

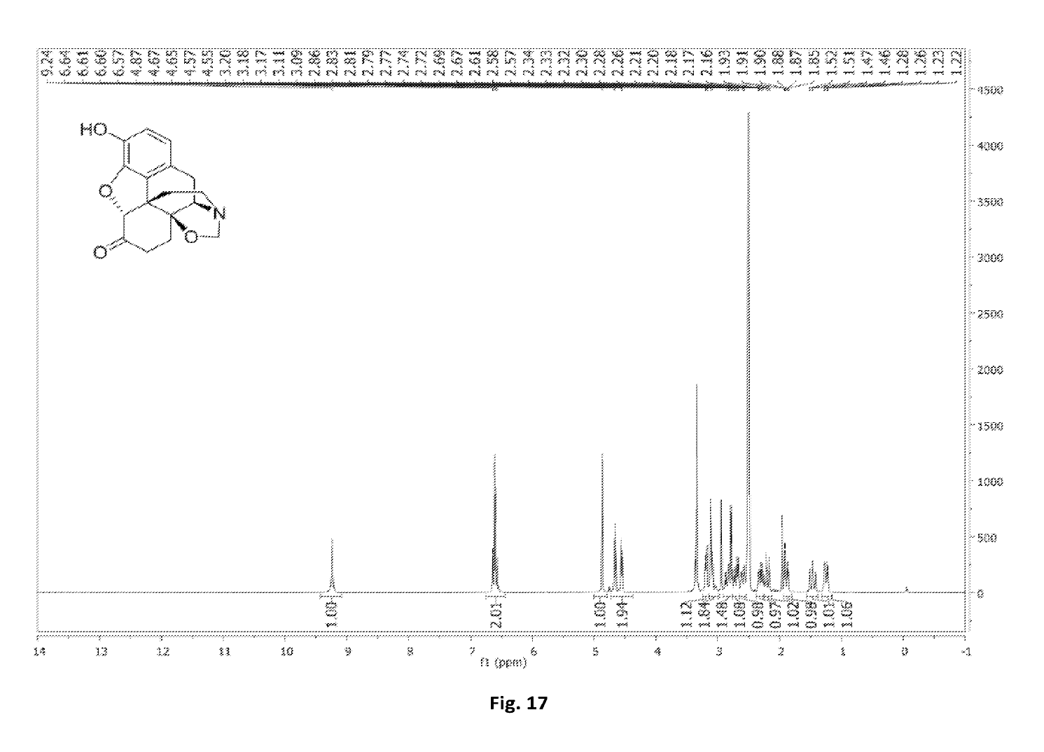

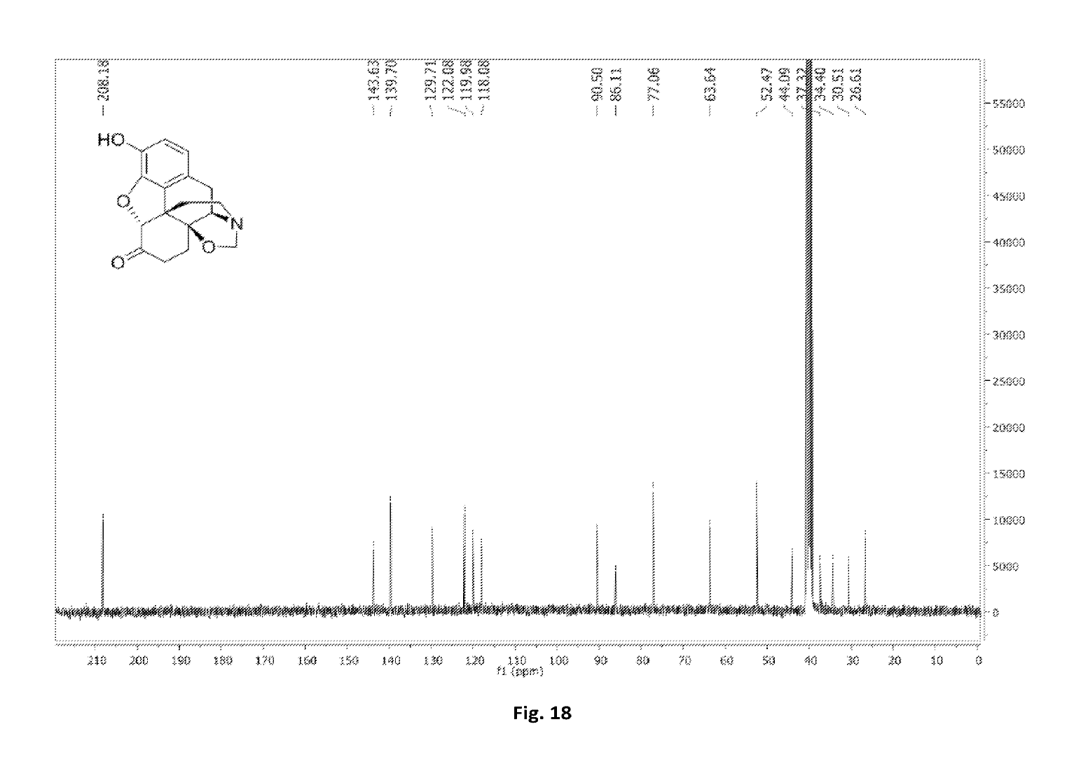

An important advantage of this methodology is that there is no need of separation of the metal catalyst after the reaction. Thus, the reaction mixture collected from the output was simply evaporated under reduced pressure until ca. 20% of its initial volume, and after addition of cold water, the desired oxazolidine 7 crystallized with 68% yield. Exemplary NMR characterization for oxazolidine 7 is shown in FIGS. 17 and 18.

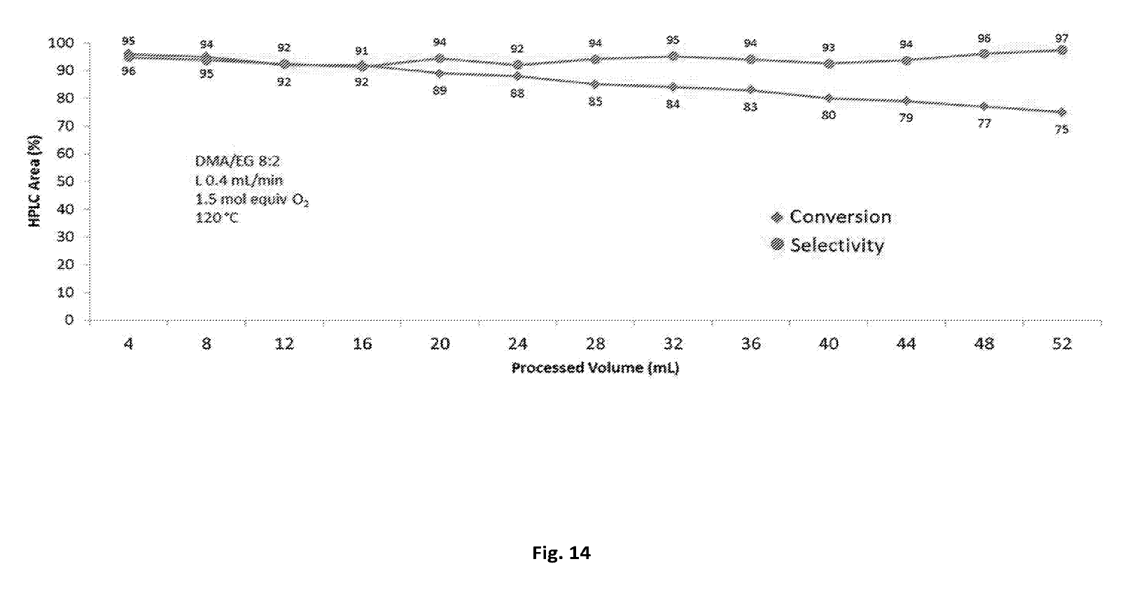

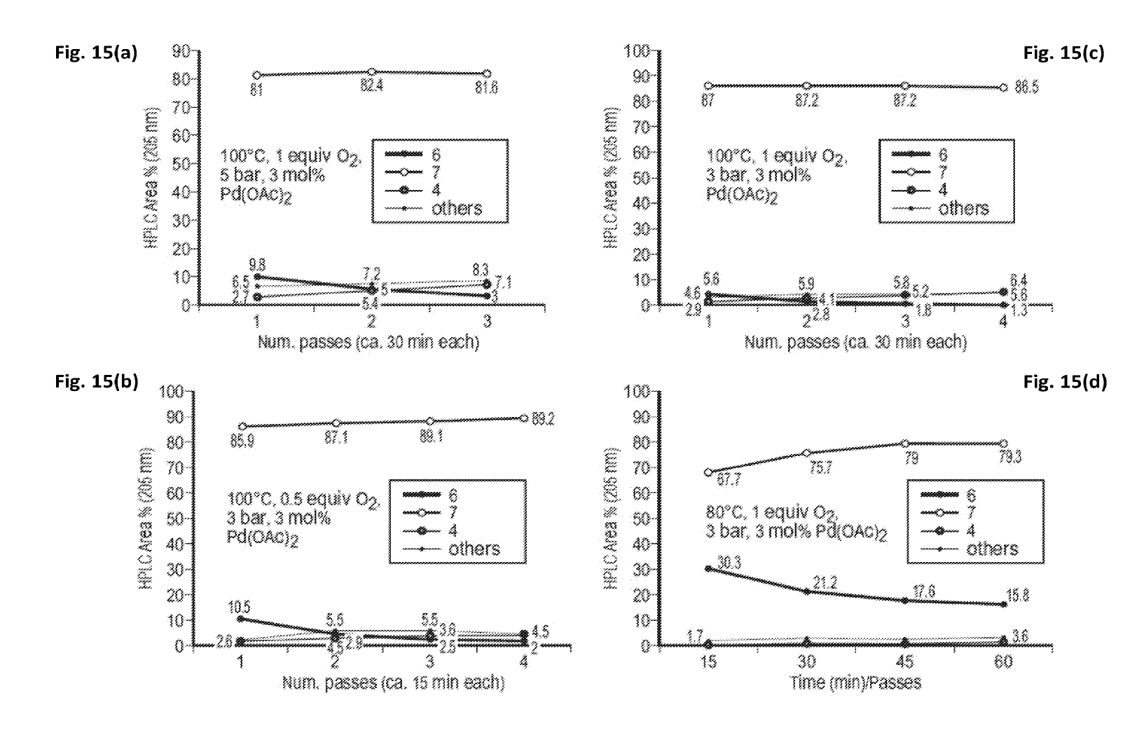

To further evaluate the catalyst stability a longer run (50 mL) was performed using the same catalyst cartridge. HPLC monitoring of the crude reaction mixture revealed a gradual decrease in the reaction conversion (FIG. 14), going below 90% after 15 mL had been processed. HPLC monitoring of the continuous flow oxidation of 6 to 7 was performed under various conditions, as shown in FIGS. 15(a), 15(b), 15(c), and 15(d).

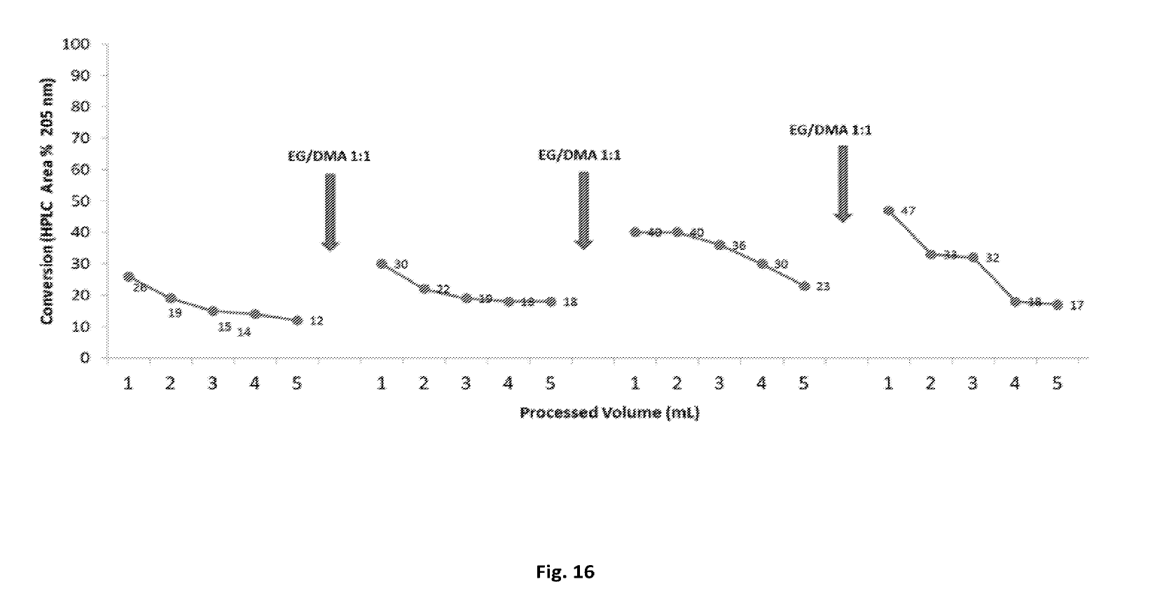

As shown in FIG. 16, HPLC monitoring for the continuous oxidation of 6 to 7 was performed using a packed bed reactor containing 220 mg SiliaCat DPP-Pd and DMA as solvent. A rapid decrease in the reaction conversion is observed in each flow run, but the catalytic efficiency can be recovered by treating the solid support with EG/DMA 1:1.

To assess whether the drop on catalytic efficiency was due to catalyst leaching from the solid support or inefficient reactivation of the Pd(0) by EG, the crude reaction mixture was analyzed by ICP-MS. The results revealed that the crude solution contained 46.4 ppm Pd, and a total amount of 2.1 mg Pd had been leached from the solid support. This value corresponds to 10% of the total amount of Pd initially contained in the immobilized catalyst. The data indicates that metal leaching does occur during the oxidation of oxymorphone 6 to 7, although the amount of metal leached is not significantly high and the procedure presented herein can be very useful for small scale reactions.

Experimental Section. 1H-NMR spectra were recorded on a Bruker 300 MHz instrument. ¹³C-NMR spectra were recorded on the same instrument at 75 MHz. Chemical shifts (6) are expressed in ppm downfield from TMS as internal standard. The letters s, d, t, q and m are used to indicate singlet, doublet, triplet, quadruplet, and multiplet. Analytical HPLC-UV/Vis (Shimadzu LC20) analysis was carried out on a C18 reversed-phase (RP) analytical column (150×4.6 mm, particle size 5 am) at 37° C. using a mobile phase A (water/acetonitrile 90:10 (v/v)+0.1% TFA) and B (MeCN+0.1% TFA) at a flow rate of 1.5 mL/min (the following gradient was applied: linear increase from solution 3% B to 100% B in 17 min). Low-resolution mass spectra were obtained on a Shimadzu LCMS-QP2020 instrument using electrospray ionization (ESI) in positive or negative mode. All solvents and chemicals were obtained from standard commercial vendors and were used without any further purification. The Pd(OAc)₂ was purchased from Aldrich (SKU: 683124).

ICP MS measurements. Liquid samples were diluted 1:100, and quantitatively determined at m/z 105 with an Agilent 7500ce inductively coupled plasma mass spectrometer. A calibration was performed with an external calibration curve established from 1.000 g of Pd/L standard (CPI International). Indium served as the internal standard.

Derivatization for HPLC analysis. To an HPLC vial containing 1 mL of saturated aqueous solution NaHCO₃ were added 50 μL of the crude reaction mixture and 200 μL Ac₂O. The vial was capped, the septum pierced with a needle, and the mixture stirred vigorously at room temperature for 20 min. The content of the vial was then directly analyzed by HPLC using the method described herein.

Preparation of oxymorphone 6. A suspension of 14-hydroxymorphinone 1 (750 mg, 2.5 mmol) and 10% Pd/C (25 mg, 1 mol %) in DMA was stirred under H₂ atmosphere for 1 h. Then, the reaction mixture was filtered using a syringe filter (PTFE, 0.45 m) and the filtrate evaporated under reduced pressure.

Continuous-flow Setup. The flow reactor consisted of an HPLC pump for introducing the liquid feed (Uniqsis Pump Module). Oxygen gas from a gas cylinder was fed into the system via a mass flow controller (Bronkhorst EL-Flow). The liquid and gaseous streams were combined in a T-mixer (stainless steel). The T-mixer was connected to the residence tube reactor via fluoropolymer tubing (PFA, 1/16″ o.d., 0.8 mm i.d.). The PFA tubing allowed visual observation of the flow profile. The residence coil reactor (FEP, ⅛″ o.d., 1.6 mm i.d., 28 mL) was heated in a GC oven to the desired temperature. The reaction mixture finally exited the system through a short cooling loop (stain less steel, 1/16″ o.d., 1 mm i.d.) and a back-pressure regulator (adjustable back-pressure regulator, Vapourtec). Pressure sensors were integrated into the T-mixer and directly after the pump.

Sequential hydrogenation/oxidative cyclization of 14-hydroxymorphinone using colloidal Pd(0). Pd(OAc)₂ (3 mol %) and AcOH (3 equiv) were dissolved in DMA (5 mL) and placed in a 25 mL two-necked round-bottom flask. The mixture was heated at 120° C. for 15 min. Formation of Pd(0) could be visually observed. Upon cooling, 14-hydroxymorphinone (150 mg, 0.5 mmol) was added and the mixture stirred under H₂ atmosphere for 1 h. Then, the crude reaction mixture was introduced into the flow reactor via an injection loop. The flow reactor had been stabilized by pumping DMA at a flow rate of 0.75 mL/min and O₂ with a flow rate of 1.7 mL/min (gas flow at normal conditions, i.e. T_{N}=0° C. and p_{N}=1 atm). The temperature of the GC-oven had been set to 120° C. and the pressure adjusted to 5 bar. After a residence time of approximately 20 min the processed product solution left the system. The reaction mixture collected from the reactor output was evaporated under reduced pressure until ca. 20% of the initial volume. 10 mL of cold water were added, and the solid obtained filtered and dried under vacuum at 50° C. (115 mg, 77%).

Sequential hydrogenation/oxidative cyclization of 14-hydroxymorphinone using heterogeneous catalysis. A suspension of 14-hydroxymorphinone (150 mg, 0.5 mmol) and 10% Pd/C (10 mg, 1 mol %) in 5 mL of DMA/ethylene glycol 9:1 was stirred at room temperature under H₂ atmosphere for one hour. The mixture was filtered (0.45 m pore size filter) and the clear solution was introduced into the flow reactor via an injection loop. The flow reactor had been stabilized by pumping DMA/ethylene glycol 8:2 at a flow rate of 0.4 mL/min and O₂ with a flow rate of 2.05 mL/min (gas flow at normal conditions, i.e. T_{N}=0° C. and p_{N}=1 atm). The temperature of the packed-bed reactor had been set to 120° C. and the pressure adjusted to 5 bar. After a residence time of approximately 20 min the processed product solution left the system. The reaction mixture collected from the reactor output was evaporated under reduced pressure until ca. 20% of the initial volume. 10 mL of cold water were added, and the solid obtained filtered and dried under vacuum at 50° C. (102 mg, 68%).

### (5aR,6R,8aS,8a¹S,11aR)-2-hydroxy-5,5α,9,10-tetrahydro-7H-6,8a¹-ethanofuro[2′,3′,4′,5′:4,5]phenanthro[9,8a-d]oxazol-11(11aH)-one

¹H NMR (300 MHz, DMSO-D₆) δ 9.24 (s, 1H), 6.60 (q, J=8.1 Hz, 2H), 4.87 (s, 1H), 4.61 (dd, J=32.1, 5.5 Hz, 2H), 3.25-3.06 (m, 3H), 2.91-2.55 (m, 3H), 2.38-2.24 (m, 1H), 2.20 (dt, J=7.3, 4.0 Hz, 1H), 1.89 (dt, J=7.3, 4.0 Hz, 1H); 1.47 (td, J=14.8, 2.9 Hz, 1H), 1.25 (dd, J=12.4, 4.0 Hz, 1H). ¹³C NMR (75 MHz, DMSO) δ 208.2, 143.6, 139.7, 129.7, 122.1, 120.0, 118.1, 90.5, 86. 1, 77.1, 63.6, 52.5, 44.1, 37.3, 34.4, 30.5, 26.6.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### REFERENCES

1. D. S. Fries in Foye's Principles of Medicinal Chemistry, 5th ed., (Eds.: D. A. Williams, T. L. Lemke), Lippincott Williams & Wilkins, Philadelphia, 2002.
2. (a) P. X. Wang, T. Jiang, G. L. Cantrell, D. W. Berberich, B. N. Trawick, T. Osiek, S. Liao, F. W. Moser, J. P. McClurg (Mallinckrodt Inc.), US20090156818, 2009; (b) P. X. Wang, T. Jiang, G. L. Cantrell, D. W. Berberich, B. N. Trawick, S. Liao (Mallinckrodt Inc.), US 20090156820, 2009; (c) S. Hosztafi, S. Makleit, Synth. Commun., 1994, 24, 3031-3045; (d) A. Ninan, M. Sainsbury, Tetrahedron, 1992, 48, 6709-6716.
3. (a) S. Hosztafi, C. Simon, S. Makleit, Synth. Commun., 1992, 22, 1673-1682; (b) H. Yu, T. Prisinzano, C. M. Dersch, J. Marcus, R. B. Rothman, A. E. Jacobson, K. C. Ricca, Bioorg. Med. Chem. Lett., 2002, 12, 165-168; (c) B. R. Selfridge, X. Wang, Y. Zhang, H. Yin, P. M. Grace, L. R. Watkins, A. E. Jacobson, K. C. Rice, J. Med. Chem., 2015, 58, 5038-5052; (d) J. Marton, S. Miklos, S. Hosztafi, S. Makleit, Synth. Commun., 1995, 25, 829-848; (e) H. S. Park, H. Y. Lee, Y. H. Kim, J. K. Park, E. E. Zvartauc, H. Lee., Bioorg. Med. Chem. Lett., 2006, 16, 3609-3613.
4. (a) M. Ann, A. Endoma-Arias, D. P. Cox. T. Hudlicky, Adv. Synth. Catal., 2013, 355, 1869-1873; (b) G. Kok, T. D. Asten, P. J. Scammells, Adv. Synth. Catal., 2009, 351, 283-286; (c) Z. Dong, P. J. Scammells, J. Org. Chem., 2007, 72, 9881-9885; (d) T. Rosenau, A. Hofinger, A. Potthast, P. Kosma, Org. Lett., 2004, 6, 541-544; (e) D. D. D. Pham, G. F. Kelso, Y. Yang, M. T. W. Hearn, Green Chem., 2012, 14, 1189-1195; (f) D. D. Do Pham, G. F. Kelso, Y. Yang, M. T. W. Hearn, Green Chem., 2014, 16, 1399-1409; (g) Y. Li, L. Ma, F. Jia, Z. Li, J. Org. Chem., 2013, 78, 5638-5646.
5. R. J. Carroll, H. Leisch, E. Scocchera, T. Hudlicky, D. P. Cox, Adv. Synth. Catal., 2008, 350, 2984-2992.
6. B. Gutmann, U. Weigl, D. Philipp Cox, C. Oliver Kappe, Chem. Eur. J., 2016, 22, 10393-10398.
7. B. Gutmann, P. Elsner, D. P. Cox, U. Weigl, D. M. Roberge, C. O. Kappe, ACS Sust. Chem. Eng., 2016, 4, DOI: 10.1021/acssuschemeng.6b01371.
8. J. G. de Vries, Dalton Trans., 2006, 421-429.
9. (a) P. N. Rylander, Hydrogenation Methods, Academic Press, New York, 1990; (b) P. N. Rylander, Catalytic Hydrogenation in Organic Synthesis, Academic Press, New York, 1979; (c) S. Nishimura, Handbook of Heterogeneous Catalytic Hydrogenation for Organic Synthesis, Wiley-Interscience, New York, 2001.
10. R. Ciriminna, V. Pandarus, A. Fidalgo, L. M. Ilharco, F. Beland, M. Pagliaro, Org. Process Res. Dev., 2015, 19, 755-768.
11. R. Greco, W. Goessler, D. Cantillo, C. O. Kappe, ACS Catal., 2015, 5, 1303-1312.

1. A process for the preparation of a compound of Formula I 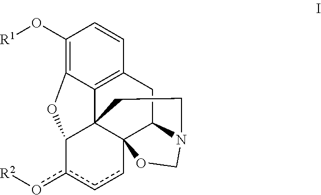
wherein
each [symbol] represents a single or double bond; provided that two double bonds are not adjacent to each other;
R¹ is selected from the group consisting of H, C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;
R² is selected from the group consisting of C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;
provided that when “ [symbol] ” represents O═, then R² is absent;
wherein one or more hydrogen atoms on the R¹ and R² groups is optionally replaced with F;
comprising 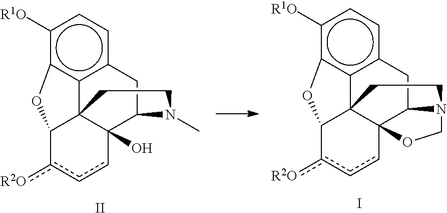
reacting a compound of Formula II with an oxidizing agent; in the presence of a metal catalyst; in a solvent or mixture of solvents; to yield the corresponding compound of Formula I.

2. The process as in claim 1, wherein the oxidizing agent is selected from the group consisting of oxygen gas and a peroxide.

3. The process as in claim 1, wherein the oxidizing agent is oxygen gas; wherein the oxygen gas is added in an amount in the range of from about 1.0 to about 5.0 equivalents, at a pressure in the range of from about 1 bar to about 20 bar; and wherein the oxygen gas is added to a mixture comprising the metal catalyst, the solvent or mixture of solvents, and the compound of Formula II.

4. The process as in claim 1, wherein the metal catalyst is selected from the group consisting of platinum, palladium, ruthenium, iron, tungsten, vanadium, iridium, copper, and gold.

5. (canceled)

6. The process as in claim 1, wherein the solvent or mixture of solvents comprises one or more of dimethylsulfoxide, N-methylpyrrolidone, dimethylacetamide, and dimethylformamide.

7. The process as in claim 1, wherein the metal catalyst is a palladium or platinum catalyst configured to exist in either a +2 oxidation state or a 0 oxidation state, the reaction occurring in the presence of an alcohol configured to convert the palladium or platinum catalyst from a +2 oxidation state to a 0 oxidation state.

8. The process as in claim 7, wherein the alcohol is a C₁₋₁₀ primary or secondary alcohol.

9. (canceled)

10. The process as in claim 1, wherein the oxidizing agent is oxygen gas and wherein the metal catalyst is palladium, wherein the solvent or mixture of solvents comprises dimethylacetamide, and wherein the reaction of the compound of Formula II with the oxidizing agent is run at a temperature in the range of from about 120° C. to about 140° C.

11. The process as in claim 1, wherein the oxidizing agent is a peroxide.

12. The process as in claim 11, wherein the peroxide is t-butylperoxide.

13. The process as in claim 11, wherein the peroxide is present in an amount in the range of from about 1 to about 3 molar equivalents.

14. The process as in claim 1, wherein the oxidizing agent is t-butylperoxide that is present in an amount in the range of from about 1 to about 3 molar equivalents; and wherein the reaction is carried out at a temperature in the range of from about 50° C. to about 100° C.

15-16. (canceled)

17. A process for the preparation of a compound of Formula III, comprising 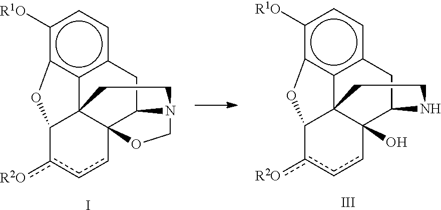
hydrolyzing a compound of Formula I under basic or acidic conditions; to yield the corresponding compound of Formula III;
wherein
each [symbol] represents a single or double bond; provided that two double bonds are not adjacent to each other;
R¹ is selected from the group consisting of H, C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;
R² is selected from the group consisting of C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;
provided that when “ [symbol] ” represents O═, then R² is absent; and
wherein one or more hydrogen atoms on the R¹ and R² groups is optionally replaced with F.

18. The process as in claim 17, wherein the hydrolysis is carried out under acidic conditions.

19. The process as in claim 17, wherein the compound of Formula I is reacted with an acid selected from the group consisting of acetic acid, hydrochloric acid, and sulfuric acid.

20. The process as in claim 17, wherein the compound of Formula I is reacted with acetic acid that is present in an amount in the range of from about 2 to about 5 molar equivalents.

21. The process as in claim 17, wherein the compound of Formula I is reacted with acetic acid that is present in an amount in the range of from about 2 to about 5 molar equivalents; at a pressure in the range of from about 10 mbar to about 1000 mbar; at a temperature in the range of from about 50° C. to about 120° C.

22. A process for the preparation of a compound of Formula VI, comprising 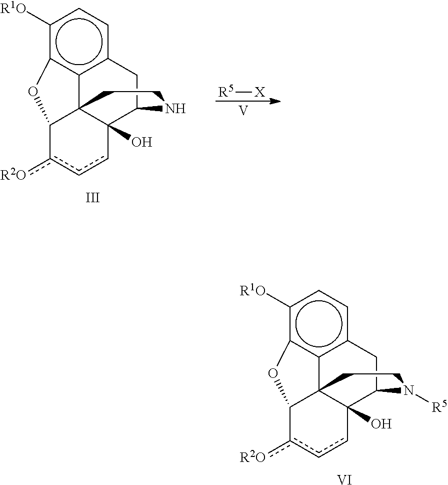
reacting a compound of Formula III with a compound of Formula V, in the presence of a base; to yield the corresponding compound of Formula VI;
wherein
each [symbol] represents a single or double bond; provided that two double bonds are not adjacent to each other;
R¹ is selected from the group consisting of H, C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;
R² is selected from the group consisting of C₁₋₁₀alkyl, C₆₋₁₀aryl, C₃₋₁₀cycloalkyl, C₁₋₁₀alkyleneC₆₋₁₀aryl, C₁₋₁₀alkyleneC₃₋₁₀cycloalkyl and an oxygen protecting group;
provided that when “ [symbol] ” represents O═, then R² is absent;
wherein one or more hydrogen atoms on the R¹ and R² groups is optionally replaced with F;
wherein R⁵ is selected from the group consisting of C₃₋₁₀cycloalkyl, C₃₋₁₀cycloalkenyl, C₁₋₁₀alkyl, C₂₋₁₀alkenyl, C₆₋₁₀aryl, C₁₋₁₀alkyleneC₆₋₁₀aryl and C₁₋₁₀alkyleneC₃₋₁₀cycloalkyl; and
wherein X is a leaving group (counteranion) selected from the group consisting of halo, Ms, Ts, Ns, Tf, and C₁₋₆acyl.

23. The process as in claim 22, wherein the compound of Formula III is reacted with the compound of Formula V; wherein the compound or Formula V is selected from the group consisting of allylbromide, cyclopropylmethyl bromide and cyclobutylmethyl bromide; in dimethylformamide; in the presence of a base selected from the group consisting of potassium carbonate and dipotassium phosphate; at a temperature in the range of from about 50° C. to about 100° C.

24-31. (canceled)

32. The process as in claim 1 wherein R¹ and R² are each independently selected from the group consisting of C₁₋₆alkyl, phenyl, naphthyl, indanyl, C₃₋₆cycloalkyl, C₁₋₆alkyleneC₆₋₁₀aryl, C₁₋₆alkyleneC₃₋₆cycloalkyl, and an oxygen protecting group.

33-42. (canceled)
